(19) United States
(12) Patent Application Publication
(10) Pub. No.: US 2025/0349317 A1
(43) Pub. Date: Nov. 13, 2025

(54) FLUORINE-CONTAINING ETHER COMPOUND, LUBRICANT FOR MAGNETIC RECORDING MEDIUM, AND MAGNETIC RECORDING MEDIUM

(71) Applicant: Resonac Corporation, Tokyo (JP)

(72) Inventors: Yutaka TANJI, Tokyo (JP); Tsuyoshi KATO, Tokyo (JP); Daisuke YAGYU, Tokyo (JP); Naoya FUKUMOTO, Tokyo (JP); Natsumi YOSHIMURA, Tokyo (JP)

(73) Assignee: Resonac Corporation, Tokyo (JP)

(21) Appl. No.: 18/861,341

(22) PCT Filed: Jul. 25, 2023
(86) PCT No.: PCT/JP2023/027183

§ 371 Date: Oct. 29, 2024

(30) Foreign Application Priority Data
Jul. 29, 2022 (JP) 2022-122139

(51) Int. Cl. G11B 5/725; C08G 65/22; C08G 65/331; C10M 107/38; C10N 40/18

(52) U.S. Cl. CPC G11B 5/7257; C08G 65/226; C08G 65/3312; C08G 65/3315; C10M 107/38; C10M 2213/0606; C10N 2040/18

(57) ABSTRACT

A fluorine-containing ether compound represented by the following formula. R¹—CH₂—R²[—CH₂—R²—CH₂—R²]ₓ—CH₂—R⁴ (x is an integer of 1 or 2; R² is a perfluoropolyether chain; at least one of x pieces of R³'s is Formula (2-1) or Formula (2-2); and R¹ and R⁴ are each a terminal group having two to four polar groups, has 1 to 9 carbon atoms in a shortest distance between carbon atoms to which adjacent polar groups are bonded, and has an oxygen atom bonded to a methylene group to which R² is bonded)

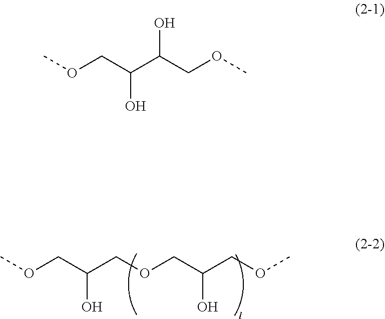

### TECHNICAL FIELD

The present invention relates to a fluorine-containing ether compound, a lubricant for a magnetic recording medium, and a magnetic recording medium.

Priority is claimed on Japanese Patent Application No. 2022-122139, filed on Jul. 29, 2022, the content of which is incorporated herein by reference.

### BACKGROUND ART

Development of a magnetic recording medium suitable for a high recording density has been advanced in order to increase a recording density in a magnetic recording and reproducing device.

In the related art, as the magnetic recording medium, there is a magnetic recording medium in which a recording layer is formed on a substrate and a protective layer made of carbon and the like is formed on the recording layer. The protective layer protects the information recorded in the recording layer and also improves the slidability of a magnetic head. However, the durability of the magnetic recording medium is not sufficiently obtained simply by providing the protective layer on the recording layer. Therefore, a lubricant is generally applied to a surface of the protective layer to form a lubricating layer.

As a lubricant used in a case of forming a lubricating layer of a magnetic recording medium, for example, a lubricant that contains a compound having a polar group such as a hydroxy group and an amino group at a terminal of a fluorine-based polymer having a repeating structure including —CF₂— has been proposed.

For example, Patent Document 1 and Patent Document 2 disclose a fluorine-containing ether compound having a skeleton where two perfluoropolyether chains are bonded through divalent linking groups having methylene groups (—CH₂—) bonded to both ends of a glycerin structure (—O—CH₂CH(OH)—CH₂—O—), in which terminal groups that are organic groups having a polar group are bonded to both terminals of the skeleton through the methylene groups.

In addition, Patent Documents 3 to 5 disclose a fluorine-containing ether compound having a skeleton where two perfluoropolyether chains are bonded to each other through divalent linking groups including a methylene group (—CH₂—) and a group (—CH(OH)—) obtained by substituting one of hydrogen atoms of a methylene group with a hydroxy group, and having two hydroxy groups, in which terminal groups that are organic groups having a polar group are bonded to both terminals of the skeleton through the methylene groups.

In addition, Patent Document 6 discloses a fluorine-containing ether compound having a skeleton where two or three perfluoropolyether chains are bonded to each other through divalent linking groups including a methylene group (—CH₂—) and a group (—CH(OH)—) obtained by substituting one of hydrogen atoms of a methylene group with a hydroxy group, and having two hydroxy groups, in which terminal groups that are organic groups having a polar group are bonded to both terminals of the skeleton through the methylene groups.

Furthermore. Patent Documents 7 and 8 disclose a fluorine-containing ether compound having a skeleton where three perfluoropolyether chains are bonded to each other through divalent linking groups including a methylene group (—CH₂—) and a group (—CH(OH)—) obtained by substituting one of hydrogen atoms of a methylene group with a hydroxy group, and having one hydroxy group, in which terminal groups that are organic groups having a polar group are bonded to both sides of the skeleton through the methylene groups.

### CITATION LIST

### Patent Documents

Patent Document 1: U.S. Pat. No. 10,540,997
Patent Document 2: PCT International Publication No. WO2021/251335
Patent Document 3: PCT International Publication No. WO2021/020066
Patent Document 4: Japanese Patent No. 6804981
Patent Document 5: Japanese Patent No. 5743438
Patent Document 6: U.S. patent Ser. No. 10/262,685
Patent Document 7: PCT International Publication No. WO2018/116742
Patent Document 8: PCT International Publication No. WO2017/145995

### SUMMARY OF INVENTION

### Technical Problem

In a magnetic recording and reproducing device, it is required to further reduce the floating amount of a magnetic head.

However, in general, in a case where the floating amount of the magnetic head is reduced, the magnetic head and a substrate are likely to collide with each other. That is, there is a tendency that the floating stability of a magnetic head is decreased. In addition, in a case where the floating amount of the magnetic head is reduced, a pickup in which the fluorine-containing ether compound in the lubricating layer adheres to the magnetic head may occur.

The present invention has been made in view of the circumstances, and an object of the present invention is to provide a fluorine-containing ether compound, which can form a lubricating layer having excellent floating stability and capable of suppressing pickup, and can thus be suitably used as a material for a lubricant for a magnetic recording medium.

Another object of the present invention is to provide a lubricant for a magnetic recording medium capable of forming a lubricating layer which includes the fluorine-containing ether compound of the present invention and has good floating stability and a high pickup suppressing effect.

Still another object of the present invention is to provide a magnetic recording medium, which has a lubricating layer including the fluorine-containing ether compound of the present invention, and has good floating stability of a magnetic head and a high pickup suppressing effect.

### Solution to Problem

The present invention includes the following aspects.

A first aspect of the present invention provides the following fluorine-containing ether compound.

[1] A fluorine-containing ether compound represented by Formula (1),

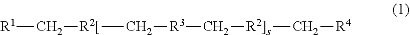

(in Formula (1), x represents an integer of 1 or 2; R² is a perfluoropolyether chain; (x+1) pieces of R²'s may be partially or entirely the same as or different from each other; R is a divalent linking group having a hydroxy group; at least one of x pieces of R³'s is a divalent linking group represented by Formula (2-1) or (2-2); in a case where x is 2, two R³'s may be the same as or different from each other; R¹ and R⁴ are each a terminal group having two to four polar groups, has 1 to 9 carbon atoms in a shortest distance between carbon atoms to which adjacent polar groups are bonded, and has an oxygen atom bonded to a methylene group to which R² is bonded; and R¹ and R⁴ may be the same as or different from each other)

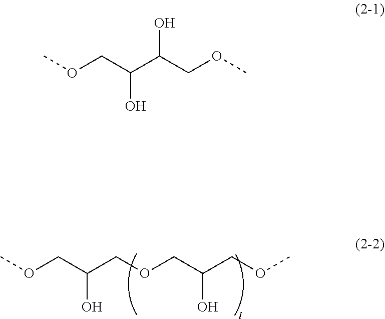

(in Formula (2-2), l represents an integer of 1 or 2)

It is preferable that the fluorine-containing ether compound of the first aspect of the present invention has the following features described in [2] to [9]. The features described in [2] to [9] are also preferably any of combinations of two or more thereof.

[2] The fluorine-containing ether compound according to [1],
in which R¹ and R⁴ in Formula (1) each independently includes at least one polar group selected from the group consisting of a hydroxy group, a cyano group, and a group having an amide bond.

[3] The fluorine-containing ether compound according to [1],
in which R¹ and R⁴ in Formula (1) are each independently a terminal group represented by any of Formulae (3-1) to (3-4).

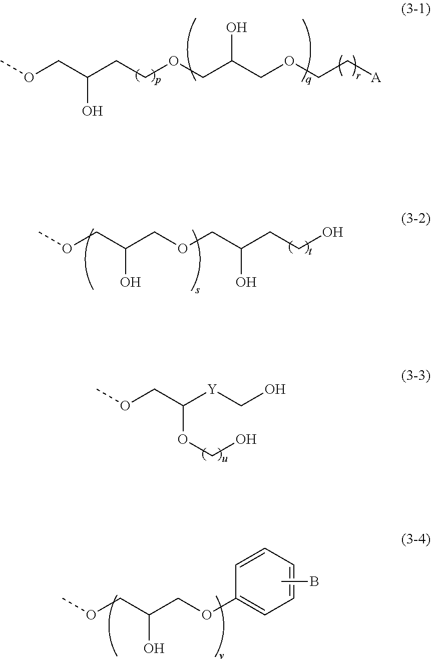

(in Formula (3-1), p represents an integer of 0 to 3, r represents an integer of 1 to 5, and a total value of p and r is 1 to 5; q represents an integer of 0 to 2; and A represents a polar group).
(in Formula (3-2), s represents an integer of 0 to 2, and t represents an integer of 1 to 5).
(in Formula (3-3), u represents an integer of 2 to 4; Y represents a linear alkylene group which may have an ether oxygen, or a single bond; and in a case where Y represents the alkylene group, a total number of carbon atoms and oxygen atoms contained in Y is 1 to 5)
(in Formula (3-4), v represents an integer of 1 to 3; and B represents a polar group in which a hydrogen at any position of a benzene ring is substituted).

[4] The fluorine-containing ether compound according to any one of [1] to [3],
in which a total number of the polar groups contained in R¹ and the polar groups contained in R⁴ in Formula (1) is 4 to 6.

[5] The fluorine-containing ether compound according to any one of [1] to [4], in which R¹ and R⁴ in Formula (1) are the same as each other.

[6] The fluorine-containing ether compound according to any one of [1] to [5], in which at least one of the x pieces of R³'s in Formula (1) is represented by Formula (2-1).

[7] The fluorine-containing ether compound according to any one of [1] to [6], in which the (x+1) pieces of R²'s in Formula (1) are each independently a perfluoropolyether chain represented by Formula (4).

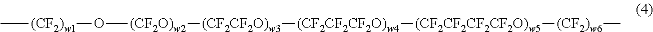

(in Formula (4), w2, w3, w4, and w5 each indicate an average degree of polymerization and each independently represent 0 to 20, provided that all of w2, w3, w4, and w5 are not (1 at the same time; w1 and w6 each indicate an average value of the number of CF₂'s and each independently represent 1 to 3; and (CF₂O), (CF₂CF₂), (CF₂CF₂CF₂O), and (CF₂CF₂CF₂CF₂ O), which are repeating units in Formula (4) are not particularly limited in the sequence order)

[8] The fluorine-containing ether compound according to any one of [1] to [6],
in which the (x+1) pieces of R²'s in Formula (1) are each independently any one selected from perfluoropolyether chains represented by Formulae (4-1) to (4-4).

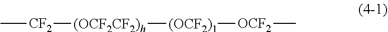

(in Formula (4-1), h and i each indicate an average degree of polymerization, h represents 1 to 20, and i represents 0 to 20)

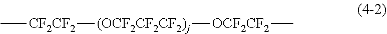

(in Formula (4-2), j indicates an average degree of polymerization and represents 1 to 15)

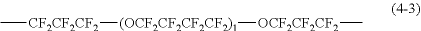

(in Formula 14.3), k indicates an average degree of polymerization and represents 1 to 10)

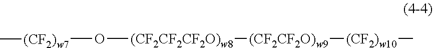

(in Formula (4-4), w8 and w9 each indicate an average degree of polymerization and each independently represent 1 to 20; and w7 and w10 are each an average value indicating the number of CF₂'s and each independently represent 1 or 2)

[9] The fluorine-containing ether compound according to any one of [1] to [8],
in which the fluorine-containing ether compound has a number-average molecular weight in a range of 500 to 10,000.

A second aspect of the present invention provides the following lubricant for a magnetic recording medium.

[10] A lubricant for a magnetic recording medium, including:
the fluorine-containing ether compound according to any one of [1] to [9].

A third aspect of the present invention provides the following magnetic recording medium.

[11] A magnetic recording medium including, in the following order, on a substrate, at least:
a magnetic layer;
a protective layer; and
a lubricating layer,
in which the lubricating layer includes the fluorine-containing ether compound according to any one of [1] to [9].

It is preferable that the magnetic recording medium according to the third aspect of the present invention has the following features described in [12].

[12] The magnetic recording medium according to [11],
in which an average film thickness of the lubricating layer is 0.5 nm to 2.0 nm.

### Advantageous Effects of Invention

The fluorine-containing ether compound of the present invention is a compound represented by Formula (1) and is suitable as a material for a lubricant for a magnetic recording medium.

Since the lubricant for a magnetic recording medium of the present invention includes the fluorine-containing ether compound of the present invention, it is possible to form a lubricating layer having cox floating stability and a high pickup suppressing effect.

The magnetic recording medium of the present invention includes a lubricating layer including the fluorine-containing ether compound of the present invention. Therefore, the magnetic recording medium of the present invention has good floating stability of a magnetic head, a high pickup suppressing effect, and excellent reliability and durability. In addition, since the magnetic recording medium of the present invention has the lubricating layer having good floating stability of a magnetic head and capable of suppressing pickup, the floating amount of the magnetic head can be further reduced.

### BRIEF DESCRIPTION OF DRAWINGS

FIG. 1 is a schematic cross-sectional view showing an embodiment of the magnetic recording medium of the present invention.

### DESCRIPTION OF EMBODIMENTS

The present inventors have conducted extensive studies as shown below in order to accomplish the objects.

In the related art, a fluorine-containing ether compound having a chain-like structure including a plurality of perfluoropolyether chains and having adsorption sites having polar groups arranged at a terminal of the chain-like structure and between the perfluoropolyether chains is preferably used as a material for a lubricant for a magnetic recording medium (which may hereinafter be simply referred to as a “lubricant”) which is applied onto a surface of a protective layer. The adsorption sites in the fluorine-containing ether compound are bonded to active points on the protective layer to improve the adhesion of the lubricating layer to the protective layer.

However, in a case where a lubricating layer of thin thickness is formed on a protective layer using the lubricant in the related art, it is difficult to realize a lubricating layer which has good floating stability of a magnetic head and is not likely to cause occurrence of pickup, as shown below.

That is, agglomerates are formed by the floating in the vicinity of the adsorption sites in the fluorine-containing ether compound included in the lubricating layer, which do not interact with the active points on the protective layer, and the smoothness of the lubricating layer is decreased. The agglomerates formed by the floating of the fluorine-containing ether compound in the lubricating layer cause a collision with the magnetic head to decrease the floating stability.

In addition, in the fluorine-containing ether compound included in the lubricating layer, adsorption sites that do not interact with an active point on the protective layer are present, and the adsorption sites are adsorbed to the magnetic head floating up in the vicinity of the adsorption sites. Then, the fluorine-containing ether compound is incorporated by the magnetic head from the adsorption sites adsorbed to the magnetic head and adheres to the magnetic head, which causes pickup.

Therefore, the present inventors have focused on the adsorption sites which can interact with the active point on the protective layer contained in the fluorine-containing ether compound included in a lubricating layer, and have conducted intensive studies as shown below to realize a fluorine-containing ether compound in which an adsorption site not interacting with an active point on a protective layer is not likely to be generated in the lubricating layer.

First, the present inventors have studied the arrangement of a plurality of adsorption sites capable of interacting with an active point on the protective layer contained in the fluorine-containing ether compound and the strength of the interaction of each adsorption site with the active point on the protective layer.

As a result, it was found that it is necessary for the fluorine-containing ether compound to have each of adsorption sites arranged at both terminals of a chain-like structure including a plurality of perfluoropolyether chains (hereinafter sometimes referred to “terminal adsorption sites”), and adsorption sites in at least one of spaces between adjacent perfluoropolyether chains (hereinafter sometimes referred to as “central adsorption sites”), in which a strength of the interaction between the terminal adsorption sites and the active points on the protective layer and a strength of the interaction between the central adsorption sites and the active points on the protective layer are appropriately balanced, and the terminal adsorption sites and the central adsorption sites can interact with the active points on the protective layer with a sufficient strength.

That is, the interaction between the terminal adsorption site and the active point on the protective layer is too strong, as compared with the interaction between the central adsorption site and the active point on the protective layer, and the active point on the protective layer with which the central adsorption sites can interact are insufficient, and thus, a central adsorption site which is not involved in the interaction with an active point on the protective layer is generated. As a result, by the floating in the vicinity of the central adsorption site, agglomerates are formed and the smoothness of the lubricating layer is decreased, leading to a decrease in floating stability. In addition, the interaction between the central adsorption site and the active point on the protective layer is too strong, as compared with the interaction between the terminal adsorption site and the active point on the protective layer, and the active point on the protective layer with which the terminal adsorption sites can interact are insufficient, and thus, a terminal adsorption site which is not involved in the interaction with an active point on the protective layer is generated, in this case, the vicinity of the terminal adsorption site floats and is thus adsorbed to the magnetic head floating tip near the terminal adsorption site on the lubricating layer, which causes pickup.

Therefore, the present inventors have conducted extensive studies on a structure of a terminal adsorption site and a central adsorption site which can interact with the active point on a protective layer with a sufficient strength, in which a balance between a strength of the interaction between the terminal adsorption site and the active point on the protective layer, and a strength of interaction between the central adsorption site and the active point on the protective layer is appropriate.

As a result, the present inventors have found that a fluorine-containing ether compound, in which a terminal group having two to four polar groups, has 1 to 9 carbon atoms in a shortest distance between carbon atoms to which adjacent polar groups are bonded, and has an oxygen atom bonded to a methylene group to which the perfluoropolyether chain is bonded, is arranged as the terminal adsorption site, and as the central adsorption site, a divalent linking group represented by Formula (2-1) or (2-2) is arranged, may be used.

The divalent linking group represented by Formula (2-1) or (2-2) is a divalent linking group having high flexibility, which has two or three hydroxy groups.

More specifically, since the perfluoropolyether chain has a sterically bulky skeleton, the hydroxy group contained in the divalent linking group arranged between adjacent perfluoropolyether chains is likely to be inhibited front being adsorbed to the protective layer by the perfluoropolyether chains which are each arranged on both neighboring sides of the divalent linking group.

For example, in a case where a divalent linking group having only one hydroxy group is arranged instead of the divalent linking group represented by Formula (2-1) or (2-2) as the central adsorption site, the hydroxy group may be inhibited from being adsorbed to the protective layer by the perfluoropolyether chains which are each arranged on both neighboring sides of the divalent linking group, which makes the divalent linking group be unable to interact with an active point on the protective layer.

In addition, in a case where a divalent linking group having a rigid structure is arranged as the central adsorption site, even in a case where the divalent linking group has a plurality of hydroxy groups, the divalent linking group may be unable to interact with an active point on the protective layer. This is because since the divalent linking group having a rigid structure cannot move freely, it may move by the linkage with the perfluoropolyether chains arranged on both neighboring sides of the divalent linking group, and may be detached from the protective layer in a case where the perfluoropolyether chains move molecularly and may move away.

In addition, in a case where a divalent linking group having less than 6 atoms that form the chain-like structure is arranged as the central adsorption site, the distance between adjacent perfluoropolyether chains is close. Therefore, the hydroxy group contained in the divalent linking group is more likely to be inhibited from being adsorbed to the protective layer by the perfluoropolyether chain, and thus, the hydroxy group contained in the divalent linking group is even less likely to be inhibited from being adsorbed to the protective layer.

On the other hand, the divalent linking group represented by Formula (2-1) or (2-2) is arranged as the central adsorption site in at least one of spaces between adjacent, perfluoropolyether chains in the fluorine-containing ether compound. The divalent linking group represented by Formula (2-1) or (2-2) has two or three hydroxy groups, and has 6 or more atoms that form the chain-like structure. Moreover, the divalent linking group represented by Formula (2-1) or (2-2) has 3 or 4 carbon atoms arranged between oxygen atoms that form an ether bond in the chain-like structure, and the both ends are bonded to the perfluoropolyether chain through a methylene group by the ether bond. Therefore, the film has moderate flexibility.

Therefore, in the fluorine-containing ether compound, the distance between adjacent perfluoropolyether chains is sufficiently secured by the divalent linking group represented by Formula (2-1) or (2-2) arranged as the central adsorption site, and it is not likely to inhibit the hydroxy group contained in the divalent linking group from being adsorbed to the protective layer by the perfluoropolyether chain. In addition, even in a case where one hydroxy group contained in the divalent linking group is inhibited from being adsorbed to the protective layer due to the bulkiness of the perfluoropolyether chain, which is each arranged on both neighboring sides of the divalent linking group represented by Formula (2-1) or (2-2), the other one or two hydroxy groups can be adsorbed to the protective layer. In addition, since the divalent linking group represented by Formula (2-1) or (2-2) has sufficient flexibility, the groups can move independently and freely without the linkage with the perfluoropolyether chains arranged on both neighboring sides of the divalent linking group even in a case where the perfluoropolyether chains move molecularly. Therefore, the interaction between the divalent linking group represented by Formula (2-1) or (2-2) and the active point on the protective layer is not likely to be affected by a molecular motion of the perfluoropolyether chains which are each arranged on both neighboring sides of the divalent linking group. Accordingly, the divalent linking group represented by Formula (2-1) or (2-2), which is the central adsorption site, is likely to be involved in the interaction with an active point on the protective layer.

However, even in the fluorine-containing ether compound in which the divalent linking group represented by Formula (2-1) or (2-2) is arranged as the central adsorption site in at least one of spaces between adjacent perfluoropolyether chains, in a case where in the fluorine-containing ether compound, terminal groups each having two to four polar groups, which are not terminal groups having 1 to 9 carbon atoms in the shortest distance between carbon atoms to which adjacent polar groups are bonded and having an oxygen atom bonded to a methylene group to which the perfluoropolyether chain is bonded, are bonded to both terminals of the chain-like structure including a plurality of perfluoropolyether chains, the terminal groups may be unable to be involved in the interaction with the active points on the protective layer.

This is presumed to be because the number of active points on the protective layer, which can interact with the fluorine-containing ether compound, is limited. That is, in the central adsorption site, the divalent linking group represented by Formula (2-1) or (2-2) capable of interacting strongly with an active point on the protective layer is arranged, and thus, the central adsorption site is preferentially likely to interact with an active point on the protective layer rather than the terminal group. As a result, it is presumed that the terminal group having a weaker adsorption force to the active point on the protective layer than the central adsorption site is not likely to be involved in the interaction with an active point on the protective layer.

On the other hand, in the fluorine-containing ether compound in which the terminal group is arranged as the terminal adsorption site and the divalent linking group represented by Formula (2-1) or (2-2) is arranged as the central adsorption site, the terminal adsorption site is a terminal group which has two to four polar groups, has an appropriate distance between adjacent polar groups, and forms an ether bond with a methylene group to which the perfluoropolyether chain is bonded. Therefore, the two to four polar groups included in the terminal adsorption site are not likely to be affected by the interaction between the adjacent polar groups and can sufficiently exert the adsorption force on the protective layer. Thus, even in a case where the divalent linking group represented by Formula (2-1) or (2-2) is arranged as the central adsorption site, the terminal adsorption site can interact with an active point on the protective layer.

Therefore, in the lubricating layer including the fluorine-containing ether compound in which a terminal group having two to four polar groups, has 1 to 9 carbon atoms in a shortest distance between carbon atoms to which adjacent polar groups are bonded, and has an oxygen atom bonded to a methylene group to which the perfluoropolyether chain is bonded, is arranged as the terminal adsorption site, and the divalent linking group represented by Formula (2-1) or (2-2) is arranged as the central adsorption site, a balance between a strength of the interaction between the terminal adsorption site and the active point on the protective layer and a strength of the interaction between the central adsorption site and the active point on the protective layer is appropriate, and the adsorption site that does not interact with an active point on the protective laver is not likely to be generated in the fluorine-containing ether compound. As a result, it is presumed that the following <1> to <3> effects are obtained.

<1> A lubricating layer, in which a decrease in smoothness of the lubricating layer due to the floating of a part of the fluorine-containing ether compound to form agglomerates can be suppressed and good floating stability is obtained, can be formed.

<2> The adsorption site in the fluorine-containing ether compound, which is not involved in the interaction with an active point on the protective layer, can be prevented from being adsorbed to the magnetic head floating in the vicinity of the adsorption site. Therefore, a lubricating layer in which a pickup starting from an adsorption site adsorbed to the magnetic head is not likely to occur can be formed.

<3> The central adsorption site and the terminal adsorption site contained in the fluorine-containing ether compound interact with an active point on the protective layer, whereby good adsorption force on the protective layer is obtained. Therefore, even in a case where the adsorption site which is not involved in the interaction with an active point on the protective layer is adsorbed to the magnetic head, the fluorine-containing ether compound is not likely to be incorporated by the magnetic head from the adsorption site adsorbed to the magnetic head, and the pickup can be suppressed.

Furthermore, the present inventors have found that by using the lubricant including the fluorine-containing ether compound, it is possible to form a lubricating layer having good floating stability and a high pickup suppressing effect, thereby leading to the present invention.

Hereinafter, preferred examples of the fluorine-containing ether compound, the lubricant for a magnetic recording medium, and the magnetic recording medium of the present invention will be described in detail. Furthermore, the present invention is not limited to embodiments shown below. In the present invention, numbers, amounts, positions, ratios, materials, configurations, and the like may be modified, added, omitted, or substituted to an extent that the gist of the present invention is maintained.

### [Fluorine-Containing Ether Compound]

The fluorine-containing ether compound of the present embodiment is represented by Formula (1)

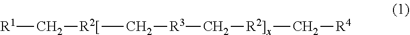

(in Formula (1), x represents an integer of 1 or 2; R² is a perfluoropolyether chain; (x+1) pieces of R²'s may be partially or entirely the same as or different from each other; R³ is a divalent linking group having a hydroxy group; at least one of x pieces of R²'s is a divalent linking group represented by Formula (2-1) or (2-2); in a case where x is 2, two R²'s may be the same as or different from each other; R¹ and R⁴ are each a terminal group having two to four polar groups, has 1 to 9 carbon atoms in a shortest distance between carbon atoms to which adjacent polar groups are bonded, and has an oxygen atom bonded to a methylene group to which R² is bonded; and R¹ and R⁴ may be the same as or different from each other)

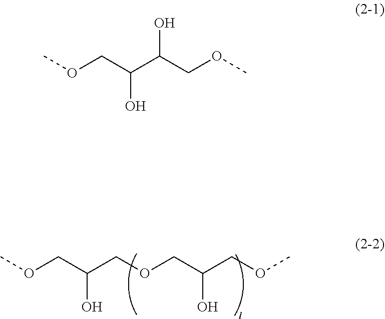

(in Formula (2-2), 1 represents an integer of 1 or 2)

As shown in Formula (1), the fluorine-containing ether compound of the present embodiment has a skeleton where the divalent linking group represented by R³ and the perfluoropolyether chain (which may hereinafter be referred to as the “PFPE chain”) represented by R² are linked through a methylene group. The terminal group represented by R¹ is bonded to one end of the skeleton through a methylene group, and the terminal group represented by R⁴ is bonded to the other end of the skeleton through a methylene group.

In the fluorine-containing ether compound represented by Formula (1), x represents an integer of 1 or 2. Since x is an integer of 1 or 2 in the fluorine-containing ether compound represented by Formula (1), the number (x+1) of PFPE chains represented by R² is 2 or 3, and unlike a compound having one PFPE chain represented by R², a divalent linking group having a hydroxy group represented by R³ is arranged between adjacent R²'s. Therefore, a lubricating layer having more excellent adhesion to the protective layer is obtained, as compared with a compound in which the number of the PFPE chains represented by R² is 1. In addition, the molecule is not too large and can move freely, as compared with a compound in which the number of the PFPE chains represented by R² is 4 or more. Therefore, a lubricating layer which is likely to wet-spread on the protective layer and has an even film thickness is easily obtained. In addition, since x is an integer of 1 or 2, the number of R³'s arranged between R²'s is 1 or 2. Thus, the number of the hydroxy groups contained in —R²[—CH₂—R³—CH₂—R²]ₓ— in Formula (1) is likely to be appropriate, and a lubricating layer having good adhesion to the protective layer is likely to be obtained. In addition, in the fluorine-containing ether compound represented by Formula (1), for example, as compared with a case where x is 3 or more, the interaction between the polar groups in the molecule can be prevented and the polar groups contained in the fluorine-containing ether compound are not likely to be aggregated. Therefore, a lubricating layer having good floating stability and a high pickup suppressing effect can be formed.

### (Divalent Linking Group Represented by R³)

In the fluorine-containing ether compound represented by Formula (1), x pieces of R³'s each independently represent a divalent linking group having a hydroxy group. In the fluorine-containing ether compound represented by Formula (I), since R³ has a hydroxy group, in a case where a lubricant including the fluorine-containing ether compound is used to form a lubricating layer on the protective layer, a suitable interaction occurs between the lubricating layer and the protective layer.

In the fluorine-containing ether compound represented by Formula (1), at least one of x pieces of R³'s is the divalent linking group represented by Formula (2-1) or (2-2). That is, in a case where x is 1, R¹ is the linking group represented by Formula (2-1) or (2-2). In a case where x is 2, one of the two R³'s or the both R³'s are the divalent linking group represented by Formula (2-1) or (2-2).

Both the divalent linking groups represented by Formulae (2-1) and (2-2) have oxygen atoms at both terminals of a chain-like structure, and the oxygen atom is bonded to the methylene group bonded to the divalent linking group represented by R¹ through an ether bond. In the divalent linking group represented by Formula (2-1), the number of carbon atoms arranged between oxygen atoms that form an ether bond in the chain-like structure is 4, and in the divalent linking group represented by Formula (2-2), the number of carbon atoms arranged between adjacent oxygen atoms that form an ether bond in the chain-like structure is 3. The oxygen atom included in the chain-like structure of the divalent linking groups represented by Formulae (2-1) and (2-2) forms an ether bond to impart moderate flexibility to the fluorine-containing ether compound represented by Formula (1), and to increase the affinity between the hydroxy group contained in the divalent linking groups represented by Formulae (2-1) and (2-2) and the protective layer. In addition, since the divalent linking groups represented by Formulae (2-1) and (2-2) have sufficient flexibility, the groups can move independently and freely without the linkage with the perfluoropolyether chains arranged on both neighboring sides even in a case where the perfluoropolyether chains move molecularly. Therefore, the divalent linking group represented by Formula (2-1) or (2-2) is not likely to be affected by the molecular motion of the perfluoropolyether chain bonded to each of both neighboring sides thereof through a methylene group, and is more likely to be involved in the interaction with an active point on the protective layer.

The divalent linking groups represented by Formulae (2-1) and (2-2) all have 6 or more atoms that form the chain-like structure. Specifically, the divalent linking group represented by Formula (2-1) has 6 atoms that form the chain-like structure. In a case where 1 in Formula (2-2) is 1, the number of atoms that form the chain-like structure of the divalent linking group represented by Formula (2-2) is 9, and in a case where 1 is 2, the number is 13. Therefore, in the fluorine-containing ether compound, the distance between adjacent perfluoropolyether chains is sufficiently secured by the divalent linking group represented by Formula (2-1) or (2-2), and the hydroxy group contained in the divalent linking group is not likely to be inhibited from being adsorbed to the protective layer by the perfluoropolyether chain.

The divalent linking group represented by Formula (2-1) has two hydroxy groups. The divalent linking group represented by Formula (2-2) has two or three hydroxy groups. Since the divalent linking group represented by Formula (2-1) or (2-2) has two or more hydroxy groups, the lubricating layer including the fluorine-containing ether compound has good adhesiveness (adhesion) with the protective layer. That is, even in a case where one hydroxy group in Formula (2-1) or (2-2) is inhibited from being adsorbed to the protective layer due to the bulkiness of the perfluoropolyether chains on both neighboring sides, the other one or two hydroxy groups can be adsorbed to the protective layer. In addition, since the number of the hydroxy groups contained in the divalent linking group is 3 or less, in the magnetic recording medium including the lubricating layer including the fluorine-containing ether compound, the active point on the protective layer, which can interact with the terminal groups represented by R¹ and R⁴, are insufficient, whereby the adsorption site that does not interact with an active point on the protective layer can be suppressed from being generated. Accordingly, the vicinity of the adsorption site which is not involved in the interaction with an active point on the protective layer is floated up, and it is thus possible to prevent the occurrence of pickup by adhering to the magnetic head floating tip on the lubricating layer. The number of the hydroxy groups contained in the divalent linking group is preferably 2 from the viewpoint that the occurrence of pickup can be more effectively prevented.

In the divalent linking group represented by Formula (2-1), carbon atoms to which two hydroxy groups in Formula (2-1) are bonded are directly bonded to each other. Therefore, the two hydroxy groups in Formula (2-1) can move mom flexibly than, for example, in a case where carbon atoms to which the hydroxy groups are bonded are bonded through a rigid alkylene chain consisting of a plurality of methylene groups. Therefore, even in a case where the perfluoropolyether chains arranged on both sides move molecularly, the two hydroxy groups in the divalent linking group represented by Formula (2-1) can each move independently and freely, and it is likely to maintain the interaction between the hydroxy group in the divalent linking group and the protective layer.

In addition, in the divalent linking group represented by Formula (2-1), since two hydroxy groups are in close proximity to each other, the alignment in which the two hydroxy groups are positioned in the same direction is restricted with respect to the alkylene chain including carbon atoms to which these hydroxy groups are bonded. In other words, two hydroxy groups included in the divalent linking group represented by Formula (2-1) are likely to be aligned in different directions with respect to the alkylene chain including the carbon atoms to which these hydroxy groups are bonded. Therefore, two dipoles induced by the two hydroxy groups are approximately in the opposite direction. This decreases a total of the dipole moments in the divalent linking group represented by Formula (2-1), and lowers a surface free energy of the fluorine-containing ether compound. As a result, the lubricating layer including the fluorine-containing ether compound is not likely to incorporate chemical contaminants in the environment, and has excellent chemical substance resistance, which is thus preferable.

The divalent linking group represented by Formula (2-2) includes a structure in which two or three glycerin structures (—O—CH—CH(OH)—CH₂—) are linked. Since the glycerin structure is flexible, the structure represented by Formula (2-2) in which 2 to 3 glycerin structures are linked is extremely flexible, and the hydroxy group can move flexibly. Therefore, even in a case where the perfluoropolyether chains arranged on both sides move molecularly, two or three hydroxy groups in the divalent linking group represented by Formula (2-2) can each move independently and freely, and the interaction between the hydroxy group in the divalent linking group and the protective layer is easily maintained.

In Formula (2-2), 1 represents an integer of 1 or 2. In a case where l is 1, the number of the hydroxy groups in the divalent linking group represented by Formula (2-2) is 2. Therefore, the interaction between the central portion (—R²[—CH₂—R¹—CH₂—R²]ₓ—) of the fluorine-containing ether compound represented by Formula (1) and the protective layer is not too strong, and it is possible to prevent the active point on the protective layer which the terminal portions (R¹—CH₂— and —CH₂—R) can interact with from being insufficient. Therefore, the fluorine-containing ether compound makes it possible to form a lubricating layer in which the occurrence of pickup due to the floating of the terminal portion of the fluorine-containing ether compound is further suppressed.

In a case where 1 in Formula (2-2) is 2, the number of the hydroxy groups in the divalent linking group represented by Formula (2-2) is 3. Therefore, the interaction between the central portion (—R²[—CH₂—R³—CH₂—R²]ₓ—) of the fluorine-containing ether compound represented by Formula (1) and the protective layer is strong. As a result, the floating of the central portion of the compound represented by Formula (1) front the protective layer is suppressed, the smoothness of the lubricating layer is maintained, and the floating stability is good.

In the fluorine-containing ether compound represented by Formula (1), in a case where x is 2 and only one of the two R³'s is a divalent linking group represented by Formula (2-1) or (2-2), the other R³ which is not the divalent linking group represented by Formula (2-1) or (2-2) may be a divalent linking group having a hydroxy group. It is preferable that R³, which is not the divalent linking group represented by Formula (2-1) or (2-2), has oxygen atoms at the terminals on both sides and is bonded to a methylene group bonded to the divalent linking group represented by R³ through an ether bond. The number of the hydroxy groups contained in R³, which is not the divalent linking group represented by Formula (2-1) or (2-2), can be, for example, 1 to 3, and is preferably 1 or 2. Examples of R³, which is not the divalent linking group represented by Formula (2-1) or (2-2), include a glycerin structure (—O—CH₂—CH(OH)—CH₂—O—).

In Formula (1), case where x is 2 and two R₃'s are both divalent linking groups represented by Formula (2-1) or (2-2), the two R³'s may be the same as or different from each other. In a case where the two R³'s are the same as each other, a coating state of the fluorine-containing ether compound with respect to the protective layer is more even and a lubricating layer having better adhesion can be formed. In addition, in a case where two R³'s are the same as each other, it is easy to produce the fluorine-containing ether compound, which is thus preferable.

(Terminal Groups Represented by R¹ and R⁴)

In the fluorine-containing ether compound represented by Formula (1), R¹ and R⁴ are each a terminal group having two to four polar groups, has 1 to 9 carbon atoms in a shortest distance between carbon atoms to which adjacent polar groups are bonded, and has an oxygen atom bonded to the methylene group (—CH₂—) to which R² is bonded.

In the present embodiment, since R¹ and R⁴ are each the terminal group, the lubricating layer including the fluorine-containing ether compound represented by Formula (1) has good floating stability and a high pickup suppressing effect.

More specifically, since the number of the polar groups included in each of R¹ and R⁴ is 2 or more, in a case where a lubricating layer is formed on the protective layer, using a lubricant including the fluorine-containing ether compound, a suitable interaction occurs between the lubricating layer and the protective layer. As a result, the lubricating layer has excellent adhesion to the protective layer and has a high pickup suppressing effect. In addition, since the number of the polar groups included in each of R¹ and R⁴ is 4 or less, it is possible to suppress the fluorine-containing ether compound from being aggregated into an agglomerate due to excessively high polarity of the fluorine-containing ether compound, leading to a loss of the smoothness of the lubricating layer in the lubricating layer including the fluorine-containing ether compound. The number of the polar groups included in R¹ and R⁴ is preferably 3 or less, and most preferably 2 since the fluorine-containing ether compound is not likely to be aggregated. In a case where the number of the polar groups included in R¹ and R⁴ is 2, the polar groups contained in the terminal groups included in R¹ and R⁴ can be effectively suppressed from being aggregated with each other in a magnetic recording medium having a lubricating layer including the fluorine-containing ether compound. As a result, in the fluorine-containing ether compound, R¹ and R⁴ can be prevented from floating to form agglomerates and a lubricating layer having good floating stability can be formed.

R¹ and R⁴ may be the same as or different from each other. Therefore, the number of the polar groups contained in R¹ and the number of the polar groups contained in R⁴ may be the same as or different from each other. The number of the polar groups contained in R¹ and the number of the polar groups contained in R⁴ are preferably the same as each other since the coating state of the fluorine-containing ether compound with respect to the protective layer is more even and a lubricating layer having better adhesion can be formed.

A total number of the polar groups contained in R¹ and the polar groups contained in R⁴ in Formula (1) is 4 to 8, preferably 4 to 6, more preferably 4 or 5, and most preferably 4.

In a case where the total number of the polar groups is 4 or more, the interaction between the polar group contained in R¹ and R⁴ in the fluorine-containing ether compound and the protective layer is stronger. As a result, the terminal group consisting of R¹ and R⁴, which is the adsorption site of the fluorine-containing ether compound, is separated from the protective layer, and the motion of the fluorine-containing ether compound to the magnetic head is suppressed with this portion as a starting point. Therefore, the fluorine-containing ether compound can form a lubricating layer having a high pickup suppressing effect.

In addition, in a case where the total number of the polar groups is 6 or less, polar groups that are not involved in the bonding between the lubricating layer and the active point on the protective layer are not likely to be generated. Therefore, the polar groups that are not involved in the bonding between the lubricating layer and the active point on the protective layer are aggregated to form a protrusion, and it is thus possible to prevent the collision with the magnetic head. Therefore, it is preferable that a lubricating layer having more excellent floating stability can be formed.

In addition, R¹ and R⁴ have 1 to 9 carbon atoms in the short distance between carbon atoms to which the adjacent polar group-s are bonded. Therefore, each of the polar groups contained in R¹ and R⁴ is bonded to different carbon atoms, and the carbon atoms to which the polar groups are bonded are bonded to each other through a linking group including 1 to 9 carbon atoms to which the polar groups are not bonded. Therefore, the terminal polar groups contained in R¹ and R⁴ and the polar groups adjacent to the terminal polar groups can be aligned in any case to be in close contact with the protective layer by a linking group including a carbon atom to which the polar groups are not bonded. Accordingly, in the fluorine-containing ether compound represented by Formula (1), it is presumed that the terminal group represented by R¹ and R⁴ is not likely to float to form agglomerates, and excellent floating stability is obtained.

In R¹ and R⁴, an atom other than a carbon atom may be present between carbon atoms to which adjacent polar groups are bonded. Examples of the atom other than the carbon atom include an oxygen atom and a nitrogen atom.

In addition, R¹ and R⁴ are each a terminal group having an oxygen atom bonded to the methylene group (—CH₂—) to which R² is bonded. That is, R¹ and R⁴ have an oxygen atom at an end part on the side bonded to CH₂ each adjacent to R¹ and R⁴. Oxygen atoms arranged at the end parts of R¹ and R⁴ form an ether bond (—O—) with atoms bonded to both sides thereof. This ether bond imparts moderate flexibility to the fluorine-containing ether compound represented by Formula (1), and increases the affinity between the polar group contained in the terminal group represented by R¹ and R⁴ and the protective layer. Thus, the fluorine-containing ether compound represented by Formula (1) can form a lubricating layer having excellent adhesion to the protective layer.

The polar groups contained in R¹ and R⁴ are each preferably a polar group selected from the group consisting of a hydroxy group (—OH), an amino group (—NH₂), a carboxy group (—COOH), a formyl group (—(C═O))H), a carbonyl group (—CO), a sulfo group (—SOH), a cyano group (—CN), and a group having an amide bond (—NR⁷COR⁸ or —CONR⁹R¹⁰; R⁷, R⁸, R⁹, and R¹⁰ are each independently a hydrogen atom or an organic group). In the group having an amide bond, R⁷ and R⁸ may be bonded to each other to form a ring, and R⁹ and R¹⁰ may be bonded to each other to form a ring. R³, R⁸, R⁹, and R¹⁰ in the group having an amide bond are each independently preferably selected from the group consisting of a hydrogen atom, a methyl group, an ethyl group, a propyl group, and a butyl group. As the group having an amide bond, an acetamide group (—NHC(═O)CH₃) or a carboxamide group (—C(═O)NH₂) is preferable. The two to four polar groups contained in R¹ and R⁴ may be partially or entirely the same as or different from each other.

It is preferable that R¹ and R⁴ each independently include at least one polar group selected from the group consisting of a hydroxy group, a cyano group, and a group having an amide bond. It is more preferable that R¹ and R⁴ each include at least one hydroxy group as the polar group.

The two to four polar groups contained in each of R¹ and R⁴ are preferably polar groups selected from the group consisting of a hydroxy group, a cyano group, and a group having an amide bond.

In the fluorine-containing ether compound represented by Formula (1), it is preferable that R¹ and R⁴ are each independently a terminal group represented by any of Formulae (3-1) to (3-4).

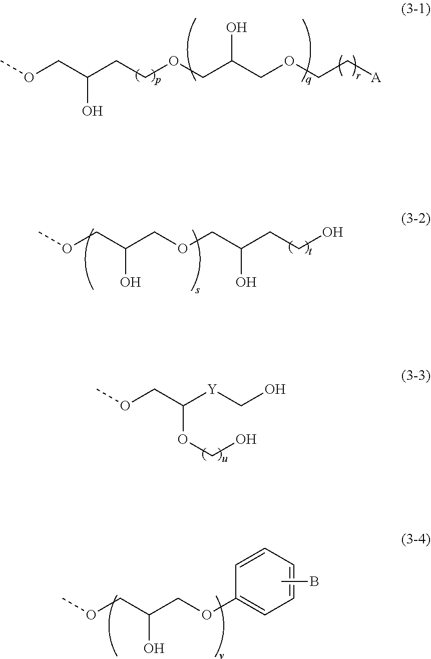

(in Formula (3-1), p represents an integer of 0 to 3, r represents an integer of 1 to 5, and a total value of p and r is 1 to 5; q represents an integer of 0 to 2; and A represents a polar group).
(in Formula (3-2), s represents an integer of 0 to 2, and t represents an integer of 1 to 5).
(in Formula (3-3), u represents an integer of 2 to 4; Y represents a linear alkylene group which may have an ether oxygen, or a single bond; and in a case where Y represents the alkylene group, a total number of carbon atoms and oxygen atoms contained in Y is 1 to 5)
(in Formula (34), v represents an integer of 1 to 3; and B represents a polar group in which a hydrogen at any position of a benzene ring is substituted).

In a case where R¹ and R⁴ are the terminal groups represented by any of Formulae (3-1) to (3-4), the total number of the polar groups contained in R¹ and the polar groups contained in R⁴ in Formula (1) (the total number of the hydroxy groups and the polar groups A in a case where R¹ and/or R⁴ is Formula (3-1), the total number of the hydroxy groups in a case where R¹ and/or R⁴ is Formula (3-2) or Formula (3-3), and the total number of the hydroxy groups and the polar groups 13 in a case where R¹ and/or R⁴ is Formula (3-4)) is 4 to 8, preferably 4 to 6, more preferably 4 or 5, and most preferably 4.

In the terminal group represented by Formula (3-1), the linking group between a carbon atom to which the polar group A arranged at a terminal is bonded and a carbon atom to which a hydroxy group adjacent to the polar group A arranged at the terminal is bonded includes an oxygen atom that forms an ether bond. p in Formula (3-1) represents an integer of 0 to 3. r represents an integer of 1 to 5, and a total value of p and r is 1 to 5. Therefore, the linking group has a linear chain-like structure consisting of 3 to 7 atoms including carbon atoms to which the polar group A and the hydroxy group are not bonded.

In the terminal group represented by Formula (3-1), since the linking group has a linear chain-like structure including an oxygen atom that forms an ether bond, and consisting of 3 or more atoms including a carbon atom to which the polar group A and the hydroxy group are not bonded, the distance between the polar group A and a hydroxy group adjacent to the polar group A is appropriate. Therefore, it is possible to suppress an interaction between the polar group A and a hydroxy group adjacent to the polar group A in the molecule, and both the polar group A and a hydroxy group adjacent to the polar group A can adhere to the protective layer. In addition, since the linking group has a linear chain-like structure consisting of 3 or more atoms, the molecular motion is appropriate and the intramolecular aggregation is not likely to occur.

In addition, since the linking group has a linear chain-like structure including an oxygen atom that forms an ether bond, and consisting of 7 or less atoms including carbon atoms to which the polar group A and a hydroxy group are not bonded, the hydrophobicity of the linking group is not excessively high to impair the adhesion to the protective layer.

Thus, the lubricating layer including the fluorine-containing ether compound, in which the linking group has a linear chain-like structure consisting of 3 to 7 atoms including carbon atoms to which the polar group A and a hydroxy group are not bonded, has excellent adhesion to the protective layer, has excellent floating stability, and has a high pickup suppressing effect.

In Formula (3-1), a total value of p and r is 1 to 5, and preferably 1 to 3. In R¹ and R⁴, the carbon atoms included in the linking group arranged between carbon atoms to which the polar groups are bonded prevent an intramolecular interaction between the polar groups in close proximity to each other from occurring preferentially to the interaction between the polar group and the protective layer, and improves the adhesion between the polar group and the protective layer in R¹ and R⁴. On the other hand, in a case where the number of carbon atoms included in the linking group is excessively large, the flexibility of the terminal group represented by R¹ and R⁴ is decreased and it may be difficult for the protective layer to be evenly coated. In the terminal group represented by R¹ and R⁴, a total value of p and r is 5 or less, and thus, the alkylene chain of the main chain portion of Formula (3-1) is not too long. Therefore, it is prevented that due to the long rigid alkylene chain, the flexibility of the terminal portion is decreased and the interaction with the protective layer is weakened, causing the terminal portion to float up. p is preferably 0 or 1, and more preferably 0. r is preferably 1 or 2, and more preferably 1.

A in Formula (3-1) represents a polar group. A is preferably a polar group selected from the group consisting of a hydroxy group (—OH), an amino group (—NH₂), a carboxy group (—COOH), a formyl group (—(C═O)H), a carbonyl group (—CO—), a sulfo group (—SO₃H), a cyano group (—CN), and a group having an amide bond (—NR⁷COR⁸ or —CONR⁹R¹⁰; R⁷, R⁸, R⁹, and R¹⁰ are each independently a hydrogen atom or an organic group). Examples of the group having an amide bond include the group having an amide bond, which is described in the description of the polar groups contained in R¹ and R⁴, and particularly, the acetamide group (—NHC(═O)CH₃) or the carboxamide group (—C(═O)N H) is preferable. As a result, the fluorine-containing ether compound can form a lubricating layer having a stronger interaction with the protective layer.

Among these polar groups. A is more preferably a polar group selected from the group consisting of the hydroxy group, the cyano group, and the group having an amide bond, and still more preferably the hydroxy group, the acetamide group, or the carboxamide group.

In a case where A in Formula (3-1) is the hydroxy group, all the polar groups in Formula (3-1) are hydroxy groups. In a case where all the polar groups contained in R¹ and R⁴ are hydroxy groups, a coating state of the fluorine-containing ether compound with respect to the protective layer is more even and a lubricating layer having better adhesion can be formed. Therefore, in a case where one of R¹ or R⁴ is Formula (3-1) and A is the hydroxy group, the other of R¹ or R⁴ is preferably any of Formula (3-1). Formula (3-2), or Formula (3-3) in which A is the hydroxy group, in a case where A in Formula (3-1) is the acetamide group or the carboxamide group, the fluorine-containing ether compound can form a lubricating layer in which the interaction with the protective layer is stronger.

In Formula (3-1), q represents an integer of 0 to 2. The number of polar groups in Formula (3-1) is q+2, and as described above, the number of the polar groups included in R¹ and R⁴ is preferably 3 or less, and most preferably 2. Therefore, q in Formula (3-1) is preferably 0 or 1, and more preferably 0.

In the terminal group represented by Formula (3-2), the linking group between a carbon atom to which the terminal hydroxy group is bonded and a carbon atom to which a hydroxy group adjacent to the terminal hydroxy group is bonded does not include an oxygen atom. t in Formula (3-2) represents an integer of 1 to 5. Therefore, the linking group has a linear chain-like structure consisting of 1 to 5 atoms including carbon atoms to which hydroxy groups are not bonded. Since the linking group included in Formula (3-2) has a linear chain-like structure consisting of one or more atoms including carbon atoms to which hydroxy groups are not bonded, the distance between the terminal hydroxy group and a hydroxy group adjacent to the terminal hydroxy group is appropriate. Therefore, it is possible to suppress the interaction between the terminal hydroxy group and a hydroxy group adjacent to the terminal hydroxy group in the molecule, and the intramolecular aggregation is not likely to occur.

in addition, as described above, in a case where the number of carbon atoms included in the linking group arranged between carbon atoms to which polar groups are not bonded is too large in R¹ and R⁴, the flexibility of the terminal groups represented by R¹ and R⁴ is decreased, and it may be difficult to evenly coat the entire surface of the protective layer. In the terminal group represented by Formula (3-2), since t is 5 or less, the alkylene chain of the main chain portion of R¹ and R⁴ is not too long. Therefore, it is prevented that due to the long rigid alkylene chain, the flexibility of the terminal portion is decreased and the interaction with the protective layer is weakened, causing the terminal portion to float up. t is preferably 4 or less, more preferably 1 or 2, and still more preferably 1.

In addition, since the linking group has a linear chain-like structure not including an oxygen atom that forms an ether bond, and consisting of 5 or less atoms including carbon atoms to which hydroxy groups are not bonded, the hydrophobicity of the linking group is not excessively high to impair the adhesion to the protective layer.

Thus, the lubricating layer including the fluorine-containing ether compound, in which the linking group has a linear chain-like structure consisting of 1 to 5 atoms, not including an oxygen atom that forms an ether bond and including carbon atoms to which hydroxy groups are not bonded, has excellent adhesion to the protective layer, has excellent floating stability, and has a high pickup suppressing effect.

in Formula (3-2), s represents an integer of 0 to 2. The number of polar groups in Formula (3-2) is s+2, and as described above, the number of the polar groups included in R¹ and R⁴ is preferably 3 or less, and most preferably 2. Therefore, s in Formula (3-2) is preferably 0 or 1, and more preferably 0.

The terminal group represented by Formula (3-3) has no secondary hydroxy group and has two primary hydroxy groups. In a case where the hydroxy group included in R¹ and/or R⁴ is a primary hydroxy group, the hydroxy group is more likely to be involved in the interaction with an active point on the protective layer, as compared to a case where the hydroxy group is a secondary hydroxy group. The terminal group represented by Formula (3-3) has only two primary hydroxy groups which do not have a secondary hydroxy group and are likely to interact with an active point on the protective layer. Therefore, case where R¹ and/or R⁴ is the terminal group represented by Formula (3-3) the fluorine-containing ether compound can form a lubricating layer having good adhesion to the protective layer.

In addition, in the terminal group represented by Formula (3-3), a linking group between carbon atoms to which two primary hydroxy groups are each bonded includes a carbon atom to which a hydroxy group is not bonded and an oxygen atom that forms an ether bond, u in Formula (3-3) represents an integer of 2 to 4. Y represents a linear alkylene group which may have an ether oxygen, or a single bond. In a case where Y is an alkylene group, a total number of carbon atoms and oxygen atoms contained in Y is 1 to 5. Therefore, the linking group has a structure consisting of 3 to 10 atoms including carbon atoms to which hydroxy groups are not bonded and an oxygen atom that forms an ether bond.

In the terminal group represented by Formula (3-3), since the linking group has a structure including an oxygen atom that forms an ether bond, and consisting of 3 or more atoms including carbon atoms to which hydroxy groups are not bonded, the distance between the two primary hydroxy groups is appropriate. Therefore, the molecular motion is appropriate and the intramolecular aggregation is not likely to occur. Therefore, the fluorine-containing ether compound can form a lubricating layer having good adhesion to the protective layer.

In addition, in the terminal group represented by Formula (3-3), since the linking group has a structure consisting of 10 or less atoms including an oxygen atom that foins an ether bond and a carbon atom to which a hydroxy group is not bonded, the hydrophobicity of the linking group is not excessively high to impair the adhesion to the protective layer.

Accordingly, the lubricating layer including the fluorine-containing ether compound, in which the linking group has a structure consisting of 3 to 10 atoms, including an oxygen atom that forms an ether bond and including carbon atoms to which hydroxy groups are not bonded, has excellent adhesion to the protective layer, has excellent floating stability, and has a high pickup suppressing effect.

In addition, since u in Formula (3-3) is 2 or more, the distance between the primary hydroxy group included in —O—(CH₂)ᵤ—OH (which may hereinafter be referred to as a “side chain portion”) in Formula (3-3) and bulky sites such as a PFPE chain represented by R² bonded to R¹ and R⁴ through a methylene group and tertiary carbon to which a side chain-like portion of Formula (3-3) is bonded is sufficiently increased. Therefore, the primary hydroxy group included in the side chain portion of Formula (3-3) can move easily and freely. In addition, since u is 4 or less, the flexibility of the side chain portion in Formula (3-3) is maintained. Therefore, the fluorine-containing ether compound has excellent adhesion to a protective layer, exhibits high floating stability, and can form a lubricating layer having a high pickup suppressing effect.

In addition, as described above, in a case where the number of carbon atoms included in the linking group arranged between carbon atoms to which polar groups are bonded in R¹ and R⁴ is too large, the flexibility of the terminal groups represented by R¹ and R⁴ is decreased and it may be difficult to evenly coat the entire surface of the protective layer. Therefore, u is preferably 2 or 3, and more preferably 2.

In addition, in Formula (3-3), the primary hydroxy group included in —Y—CH₂— OH is bonded to the carbon atom to which a side chain portion of Formula (3-3) is bonded, through —Y—CH₂—. Therefore, the primary hydroxy group included in —Y—CH₂—OH is easily movable, as compared with a case where the hydroxy group is directly bonded to the carbon atom, and easily involved in the interaction with an active point on the protective layer.

Since the primary hydroxy group included in —Y—CH₂—OH in Formula (3-3) is more likely to be involved in the interaction with an active point on the protective layer, it is preferable that Y in Formula (3-3) is not a single bond but a linear alkylene group which may have ether oxygen, in this case, the distance between the primary hydroxy group included in —Y—CH—OH and bulky sites such as a PFPE chain represented by R² bonded to R¹ and R⁴ through a methylene group and tertiary carbon to which a side chain-like portion of Formula (3-3) is bonded is increased, and thus, the primary hydroxy group included in —Y—CH₂—OH is more likely to move freely.

In a case where Y in Formula (3-3) is a linear alkylene group which may have ether oxygen, the total number of carbon atoms and oxygen atoms contained in Y is 1 to 5. In the terminal group represented by Formula (3-3), since the total number of carbon atoms and oxygen atoms contained in Y is 5 or less, the alkylene chain in the main chain portion in Formula (3-3) is not too long. Therefore, it is prevented that due to the long rigid alkylene chain, the flexibility of the terminal portion is decreased and the interaction with the protective layer is weakened, causing the terminal portion to float up. The total number of carbon atoms and oxygen atoms contained in Y is preferably 1 to 4, and more preferably 1 to 3. The number of carbon atoms contained in Y is preferably 1 to 3, and more preferably 1 or 2.

Specific examples of Y in a case where Y in Formula (3-3) is the linear alkylene group which may have ether oxygen include —CH₂—, —CH₂CH₂—, —CH₂CH₂CH₂—, —CH₂OCH₂—, CH₂OCH₂CH₂, —CH₂OCH₂CH₂CH₂, and —CH₂CH₂OCH—.

In the terminal group represented by Formula (3-4), a linking group between a carbon atom to which the polar group B is bonded and a carbon atom to which a hydroxy group ad jacent to the polar group B is bonded includes an oxygen atom that forms an ether bond. The linking group has a structure consisting of 3 to 5 atoms including carbon atoms to which the polar group B and a hydroxy group are not bonded. The number of atoms in the linking group refers to the number of atoms in the shortest distance between a carbon atom to which the polar group B is bonded and a carbon atom to which a hydroxy group adjacent to the polar group B is bonded.

In the terminal group represented by Formula (3-4), since the linking group has a structure consisting of 3 or more atoms including an oxygen atom that forms an ether bond and carbon atoms to which the polar group B and the hydroxy group are not bonded, the distance between the polar group B and a hydroxy group adjacent to the polar group B is appropriate. In addition, since the benzene ring is rigid, free rotation is difficult. Therefore, it is possible to suppress the interaction between the polar group B substituted on the benzene ring and a hydroxy group adjacent to the polar group B.

In addition, in a case where v in Formula (3-4) is 2 or more, the linking group between carbon atoms bonded to a plurality of adjacent hydroxy groups included in —(CH₂CH(OH)CH₂O)ᵥ— has a linear chain-like structure consisting of 3 atoms including an oxygen atom that forms an ether bond and carbon atoms to which the polar group B and a hydroxy group are not bonded. Since the linking group has a linear chain-like structure consisting of 3 atoms including carbon atoms to which the polar group B and a hydroxy group are not bonded, the distance between adjacent hydroxy groups is appropriate. Therefore, the interaction between the hydroxy groups in the molecule can be suppressed.

Thus, in the fluorine-containing ether compound having a terminal group represented by Formula (3-4, it is possible to suppress the interaction between the polar groups in the molecule. Therefore, the lubricating layer including the fluorine-containing ether compound having a terminal group represented by Formula (3-4) has excellent adhesion to the protective layer, is capable of obtaining excellent floating stability, and has a high pickup suppressing effect.

B in Formula (3-4) represents a polar group substituted at any position of the benzene ring. B is preferably a polar group selected from the group consisting of a hydroxy group (—OH), an amino group (—NH₂), a carboxy group (—COOH), a formyl group (—(C═O)H), a carbonyl group (—CO—), a sulfo group (—SOH), a cyano group (—CN), and a group having an amide bond (—NR⁷COR⁸ s or —CONR⁹R¹⁰; R⁷, R⁸, R⁹, and R¹⁰ are each independently a hydrogen atom or an organic group). Examples of the group having an amide bond include the group having an amide bond, which is described in the description of the polar groups contained in R¹ and R⁴, and particularly, the acetamide group (—NHC(═O)CH) or the carboxamide group (—C(═O)NH—) is preferable. As a result, the fluorine-containing ether compound can form a lubricating layer having a stronger interaction with the protective layer.

Among these polar groups. B is more preferably a polar group selected from the group consisting of the cyano group and the group having an amide bond, and still more preferably the cyano group, the acetamide group, or the carboxamide group.

In Formula (3-4), v represents an integer of 1 to 3. The number of polar groups in Formula (3-4) is v+1, and as described above, the number of the polar groups included in R¹ and R⁴ is preferably 3 or less, and most preferably 2. Therefore, v in Formula (3-4) is preferably 1 or 2, and more preferably 1.

R¹ and R⁴ may be the same as or different from each other. In a case where R¹ and R⁴ are the same as each other, a coating state of the fluorine-containing ether compound with respect to the protective layer is more even, and a lubricating layer having better adhesion can be formed.

### (PFPE Chain Represented by R²)

In the fluorine-containing ether compound represented by Formula (1), R² is a perfluoropolyether chain. In a case where the lubricant including the fluorine-containing ether compound of the present embodiment is applied onto the protective layer to form a lubricating layer, the PFPE chain represented by R² coats a surface of the protective layer and imparts lubricity to the lubricating layer, thereby reducing a frictional force between the magnetic head and the protective layer. The PFPE chain represented by R² is appropriately selected according to the performance required for the lubricant including the fluorine-containing ether compound.

(x+1) pieces of R²'s may be partially or entirely the same as or different from each other. It is preferable that (x+1) pieces of R²'s are entirely the same. This is because a coating state of the fluorine-containing ether compound with respect to the protective layer is more even, and the lubricating layer has better adhesion. The expression, two or more R²'s among (x+1) pieces of R²'s being the same, means that (x+1) pieces of R²'s include two or more R²'s having the same structure of the repeating units of the PFPE chain. The same R³'s may also include repeating units having the same structure but different average degrees of polymerization.

Examples of the PFPE chain represented by R² include a polymer or copolymer of perfluoroalkylene oxide. Examples of the perfluoroalkylene oxide include perfluoromethylene oxide, perfluoroethylene oxide, perfluoro-n-propylene oxide, perfluoro isopropylene oxide, and perfluoro butylene oxide.

It is preferable that (x+1) pieces of R²'s in Formula (i) are each independently, for example, a PFPE chain represented by Formula (4) derived from a polymer or copolymer of perfluoroalkylene oxide.

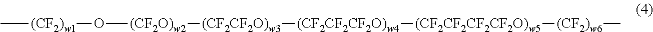

(in Formula (4), w2, w3, w4, and w5 each indicate an average degree of polymerization and each independently represent 0 to 20, provided that all of w2, w3, w4, and w5 are not 0 at the same time; w1 and w6 each indicate an average value of the number of CF₂'s and each independently represent 1 to 3; and (CF₂O), (CF₂CF₂)), (CF₂CF₂CF₂)O, and (CF₂CF₂CF₂CF₂O), which are repeating units in Formula (4) are not particularly limited in the sequence order)

In Formula (4), w2, w3, w4, and w5 indicate an average degree of polymerization, and each independently represent 0 to 20, preferably 0 to 15, and more preferably 0 to 10, w2, w3, w4, and w5 may be each 1 to 8, 2 to 6, 3 to 5, or the like.

In Formula (4), w1 and w6 are average values indicating the number of CF₂'s and each independently represent 1 to 3, w1 and w6 are determined depending on the structure of the repeating unit arranged at the end part of the chain-like structure in the PFPE chain represented by Formula (4) or the like.

(CF₂O), (CF₂CFA)), (CF₂CF₂CF₂O), and (CF₂CF₂CF₂CF₂F) in Formula (4) are repeating units. The repeating units in Formula (4) is not particularly limited in the sequence order. In addition, the number of kinds of repeating units in Formula (4) is also not particularly limited.

(x+1) pieces of R²'s in Formula (1) are each independently preferably any one selected from PIPE chains represented by Formulae (4-1) to (4-4).

In a case where (x+1) pieces of R²'s are each any one selected from the PFPE chains represented by Formulae (4-1) to (4-4), the fluorine-containing ether compound makes it possible to obtain a lubricating layer having good lubricity. In addition, case where (x+1) pieces of R³, are each any one selected front the PFPE chains represented by Formulae (4-1) to (4-4), a proportion of the number of oxygen atoms (number of ether bonds (—O—)) to the number of carbon atoms in the PFPE chain is appropriate. Therefore, the fluorine-containing ether compound has moderate hardness. Therefore, the fluorine-containing ether compound applied onto the protective layer is not likely to be aggregated on the protective layer, and a lubricating layer of even thinner thickness can be formed on the protective layer with a sufficient coating rate. In addition, since the fluorine-containing ether compound has moderate flexibility, a lubricating layer having better floating stability can be formed.

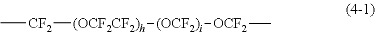

(in Formula (4-1), h and i each indicate an average degree of polymerization, h represents 1 to 20, and i represents 0 to 20)

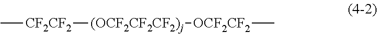

(in Formula (4-2), j indicates an average degree of polymerization and represents 1 to 15)

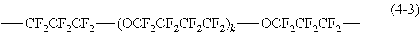

(in Formula (4-3), k indicates an average degree of polymerization and represents 1 to 10)

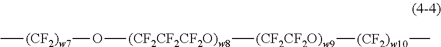

(in Formula (4-4), w8 and w9 each indicate an average degree of polymerization and each independently represent 1 to 20; and w7 and w10 are each an average value indicating the number of CF₂'s and each independently represent 1 or 2)

In Formula (4-1), (OCF₂F₂) and (OCF₂) which are the repeating units is not particularly limited in the sequence order. In Formula (4-1), the number h of (OCF₂CF₂)'s and the number i of (OCF₂)'s may be the same as or different from each other. The PFPE chain represented by Formula (4-1) may be a polymer of (OCF₂F₂). In addition, the PFPE chain represented by Formula (4-1) may be any of a random copolymer, a block copolymer, or an alternating copolymer consisting of (OCF₂CF₂) and (OCF₂).

In Formulae (4-1) to (4-3), h indicating the average degree of polymerization is 1 to 20, i is h to 20, j is 1 to 15, and k is 1 to 10, and thus, the fluorine-containing ether compound makes it possible to obtain a lubricating layer having good lubricity. In addition, in Formulae (4-1) to (4-3), h and i each indicating an average degree of polymerization are 20 or less, j is 15 or less, and k is 10 or less, and thus, the viscosity of the fluorine-containing ether compound is not excessively high, and a lubricant including the fluorine-containing ether compound is easy to apply, which is thus preferable. h, i, j, and k, each indicating an average degree of polymerization, are each preferably 1 to 10, more preferably 1.5 to 8, and still more preferably 2 to 7 since the fluorine-containing ether compound is likely to wet-spread on the protective layer and makes it possible to obtain a lubricating layer having an even film thickness.

in Formula (4-4), (CF₂CF₂CF₂O) and (CF₂CF₂O), which are the repeating units, is not particularly limited in the sequence order. In Formula (4-4), the number w8 of (CF₂CF₂CF₂O)'s and the number w9 of (CF₂CF₂O)'s, each indicating an average degree of polymerization, may be the same as or different from each other. Formula (4-4) may include any of a random copolymer, a block copolymer, and an alternating copolymer, each consisting of monomer units (CF₂CF₂CF₂O) and (CF₂CF₂O).

In Formula (4-4), w8 and w9, each indicating an average degree of polymerization, are each independently 1 to 20, preferably 1 to 15, and more preferably 1 to 10.

w7 and w10 in Formula (4-4) are average values indicating the number of CF₂'s and each independently represent 1 or 2, w7 and w10 are determined depending on the structure of the repeating unit arranged at an end part of the chain-like structure in the PFPE chain represented by Formula (4-4) or the like.

In the fluorine-containing ether compound represented by Formula (1), it is preferable that (x+1) pieces of R³'s in Formula (1) are the same as each other, x pieces of R³'s are the same as each other, and R¹ and R⁴ are the same as each other. This is because it is possible to produce the fluorine-containing ether compound easily and efficiently.

Specifically, the fluorine-containing ether compound represented by Formula (1) is preferably any of the compounds represented by Formulae (AA) to (AV) and (BA) to (BK).

In a case where the compound represented by Formula (1) is any of the compounds represented by Formulae (AA) to (AV) and (BA) to (BK), the raw materials are easily available and a lubricating layer having even better floating stability and a higher pickup suppressing effect of a magnetic recording medium can be formed even in a case where the thickness is small.

In the compounds represented by Formulae (AA) to (AV), and (BA) to (BK), Rf₁, Rf₂, and Rf₃ representing PFPE chains each have the following structure. That is, in the compounds represented by Formulae (AA) to (AP), (AS) to (AV), (BA) to (BI), and (BK), Rf₁ is the PFPE chain represented by Formula (4-1). In the compounds represented by Formulae (AQ) and (BJ), R² is the PFPE chain represented by Formula (4-2). In the compound represented by Formula (AR), Rf₃ is the PFPE chain represented by Formula (4-3). Furthermore, h and i in Rf₁, j in Rf₂, and k in Rf₃, each representing the PFPE chain in Formulae (AA) to (AV), (BA) to (BK), are values indicating an average degree of polymerization, which are thus not necessarily integers.

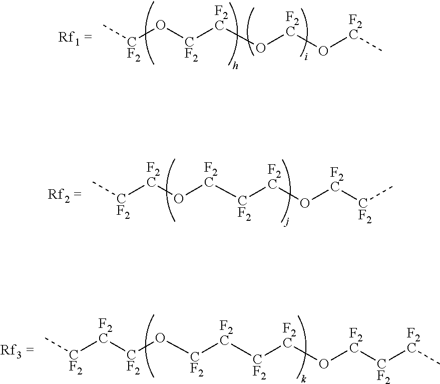

In the compounds represented by Formulae (AA) to (AV), and (BA) to (BK), at least any one of R³'s is the linking group represented by Formula (2-1) or (2-2).

In the compounds represented by Formulae (AA) to (AV), and (BA) to (BK), both R¹ and R⁴ are each the terminal group represented by any of Formulae (3-1) to (34).

In all of the compounds represented by Formulae (AA) to (AV) and (BA) to (BK), (x+1) pieces of R³'s in Formula (1) are the same as each other.

In the compounds represented by Formulae (AA) to (AM), x in Formula (1) is 1. R¹ and R⁴ are each the terminal group represented by Formula (3-1) or (3-2). R³ is the linking group represented by Formula (2-1). R² is the PFPE chain represented by Formula (4-1).

In the compounds represented by Formulae (AN) to (AP), x in Formula (1) is 1, R¹ and R⁴ are each the terminal group represented by Formula (3-1). R³ is the linking group represented by Formula (2-2). R² is the PFPE chain represented by Formula (4-1).

In the compounds represented by Formulae (AQ) and (AR), x in Formula (1) is 1. R¹ and R⁴ are each the terminal group represented by Formula (3-1). R³ is the linking group represented by Formula (2-1). The compound represented by Formula (AQ) is a compound in which R² is the PFPE chain represented by Formula (4-2). The compound represented by Formula (AR) is a compound in which R² is the PFPE chain represented by Formula (4-3).

In the compounds represented by Formulae (AS) and (AT), x in Formula (1) is 1. R¹ and R⁴ are each a terminal group represented by Formula (3-3). R³ is the linking group represented by Formula (2-1). R²'sis the PFPE chain represented by Formula (4-1).

In the compounds represented by Formulae (AU) and (AV), x in Formula (1) is 1. R¹ and R⁴ are each a terminal group represented by Formula (3-4). R³ is the linking group represented by Formula (2-1). R² is the PFPE chain represented by Formula (4-1).

In the compounds represented by Formulae (BA) to (BF), x in Formula (1) is 2. R¹ and R⁴ are each the terminal group represented by Formula (3-1) or (3-2). R³ is the linking group represented by Formula (2-1). R² is the PFPE chain represented by Formula (4-1).

In the compounds represented by Formulae (BG) and (BH), x in Formula (1) is 2. R¹ and R⁴ are each the terminal group represented by Formula (3-1). R³ is the linking group represented by Formula (2-2). R¹ is the PFPE chain represented by Formula (4-1).

In the compound represented by Formula (BI), x in Formula (1) is 2. R¹ and R⁴ are each the terminal group represented by Formula (3-1). One of the two R³'s is the linking group represented by Formula (2-1), and the other is a glycerin structure (—O—CH₂—CH(OH)—CH₂—O—). R² is the PFPE chain represented by Formula (4-1).

In the compound represented by Formula (BJ), x in Formula (1) is 2. R¹ and R⁴ are each the terminal group represented by Formula (3-1). R³ is the linking group represented by Formula (2-1). R² is the FPE, chain represented by Formula (4-2).

In the compound represented by Formula (BK), x in Formula (1) is 2. R¹ and R⁴ are each the terminal group represented by Formula (3-1). R³ is the linking group represented by Formula (2-2). R² is the PFPE chain represented by Formula (4-1).

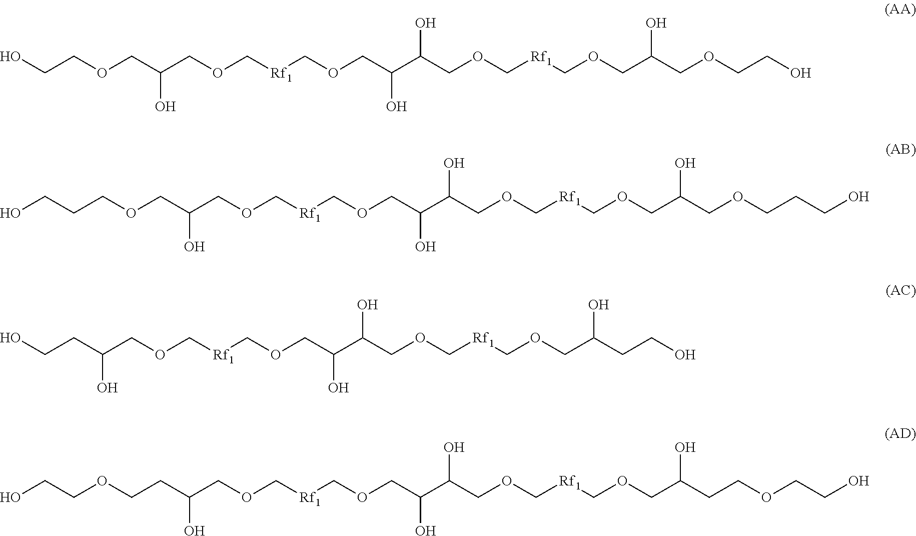

(in the two Rf₁'s in Formula (AA), h and i each indicate an average degree of polymerization, h represents 1 to 20, and i represents 0 to 20; and the average degrees of polymerization in the two Rf₁'s may be the same as or different from each other)
(in the two Rf₁'s in Formula (AB), h and i each indicate an average degree of polymerization, h represents 1 to 20, and i represents 0 to 20; and the average degrees of polymerization in the two Rf's may be the same as or different from each other)
(in the two Rf₁'s in Formula (AC), h and i each indicate an average degree of polymerization, h represents 1 to 20, and i represents 0 to 20; and the average degrees of polymerization in the two Rf₁'s may be the same as or different from each other)
(in the two Rf₁'s in Formula (AD), h and i each indicate an average degree of polymerization, h represents 1 to 20, and i represents 0 to 20; the average degrees of polymerization of the two Rf₁'s may be the same as or different from each other)

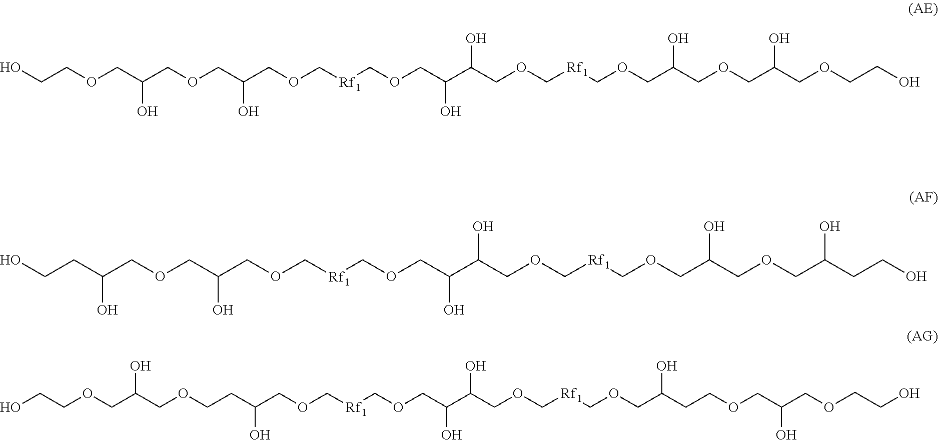

(in the two Rf₁'s in Formula (AE), h and i each indicate an average degree of polymerization, h represents 1 to 20, and i represents 0 to 20; and the average degrees of polymerization in the two Rf₁'s may be the same as or different from each other)
(in the two Rf₁'s in Formula (AF), h and i each indicate an average degree of polymerization, h represents 1 to 20, and i represents 0 to 20; and the average degrees of polymerization in the two Rf₁'s may be the same as or different from each other)
(in the two Rf₁'s in Formula (AG), b and i each indicate an average degree of polymerization, h represents 1 to 20, and i represents 0 to 20; the average degrees of polymerization of the two Rf₁'s may be the same as or different from each other)

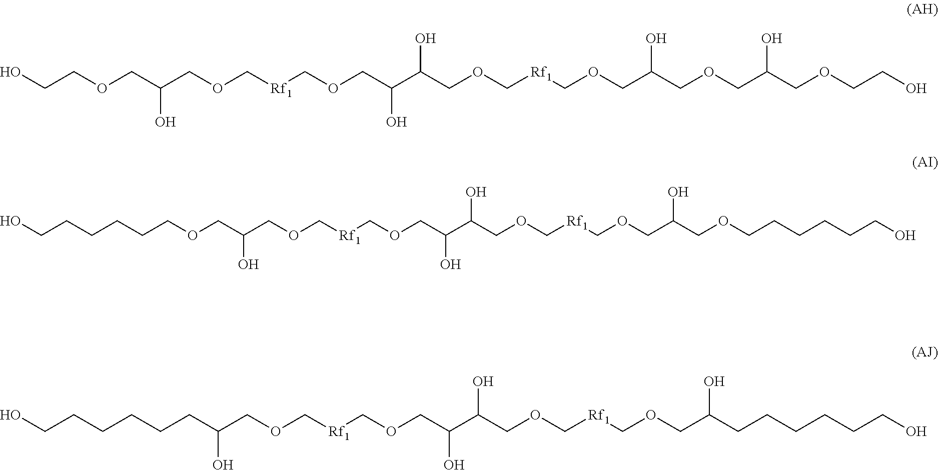

(in the two Rf₁'s in Formula (AH), h and i each indicate an average degree of polymerization, h represents 1 to 20, and i represents 0 to 20; the average degrees of polymerization of the two Rf₁'s may be the same as or different from each other)
(in the two Rf₁'s in Formula (AI), h and i each indicate an average degree of polymerization, h represents 1 to 20, and i represents 0 to 20; and the average degrees of polymerization in the two Rf₁'s may be the same as or different from each other)
(in the two Rf₁'s in Formula (AJ), h and i each indicate an average degree of polymerization, h represents 1 to 20, and i represents 0 to 20; and the average degrees of polymerization in the two Rf₃'s may be the same as or different from each other)

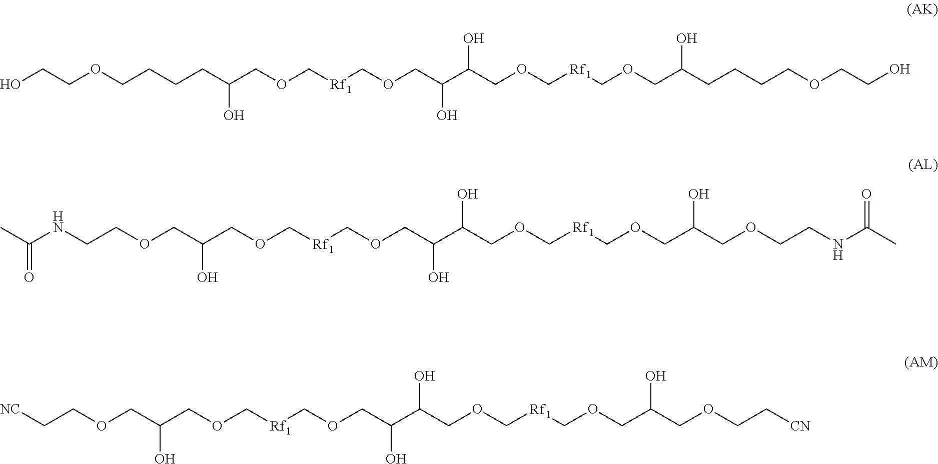

(in the two Rf₁'s in Formula (AK), h and i each indicate an average degree of polymerization, h represents 1 to 20, and i represents 0 to 20; and the average degrees of polymerization in the two Rf₁'s may be the same as or different from each other
(in the two Rf₁'s in Formula (AL), h and i each indicate an average degree of polymerization, h represents 1 to 20, and i represents 0 to 20; and the average degrees of polymerization in the two Rf₁'s may be the same as or different from each other)
(in the two Rf₃'s in Formula (AM), h and i each indicate an average degree of polymerization, h represents 1 to 20, and i represents 0 to 20; and the average degrees of polymerization in the two Rf₁'s may be the same as or different from each other)

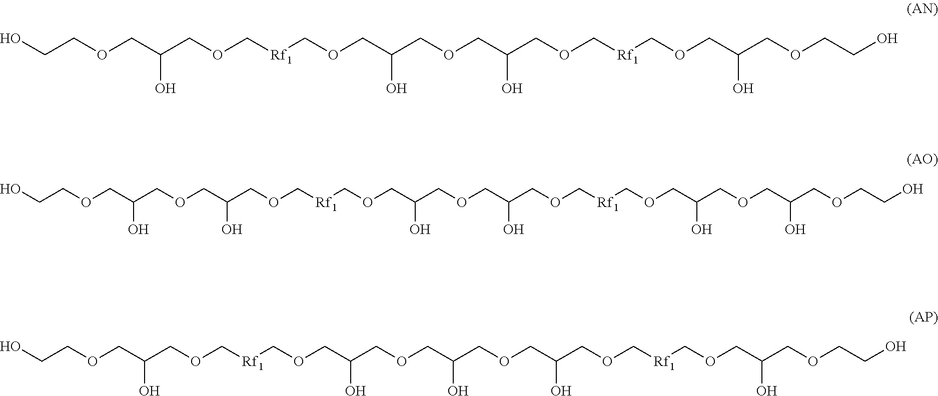

(in the two Rf₁'s in Formula (AN), h and i each indicate an average degree of polymerization, h represents 1 to 20, and i represents 0 to 20; and the average degrees of polymerization in the two Rf₁'s may be the same as or different from each other)
(in the two Rf₁'s in Formula (AO), h and i each indicate an average degree of polymerization, h represents 1 to 20, and i represents 0 to 20; the average degrees of polymerization of the two Rf₁'s may be the same as or different from each other)
(in the two Rf₁'s in Formula (AP), h and i each indicate an average degree of polymerization, h represents 1 to 20, and i represents 0 to 20; and the average degrees of polymerization in the two Rf₁'s may be the same as or different from each other)

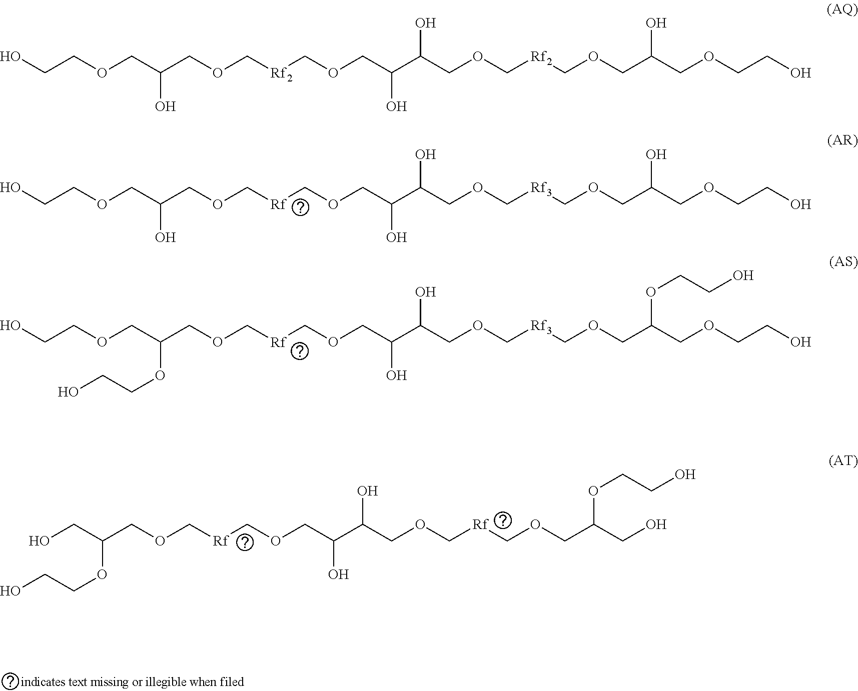

(in the two Rf₂'s in Formula (AQ), j indicates an average degree of polymerization and represents 1 to 15; and the average degrees of polymerization in the two Rf₂'s may be the same as or different from each other)
(in the two Rf₃'s in Formula (AR), k indicates an average degree of polymerization and represents 1 to 10; and the average degrees of polymerization in the two Rf₁'s may be the same as or different from each other)
(in the two Rf₁'s in Formula (AS), h and i each indicate an average degree of polymerization, h represents 1 to 20, and i represents 0 to 20; and the average degrees of polymerization in the two Rf₁'s may be the same as or different from each other)
(in the two Rf₁'s in Formula (AT), h and i each indicate an average degree of polymerization, h represents 1 to 20, and i represents 0 to 20; and the average degrees of polymerization in the two Rf₁'s may be the same as or different from each other)

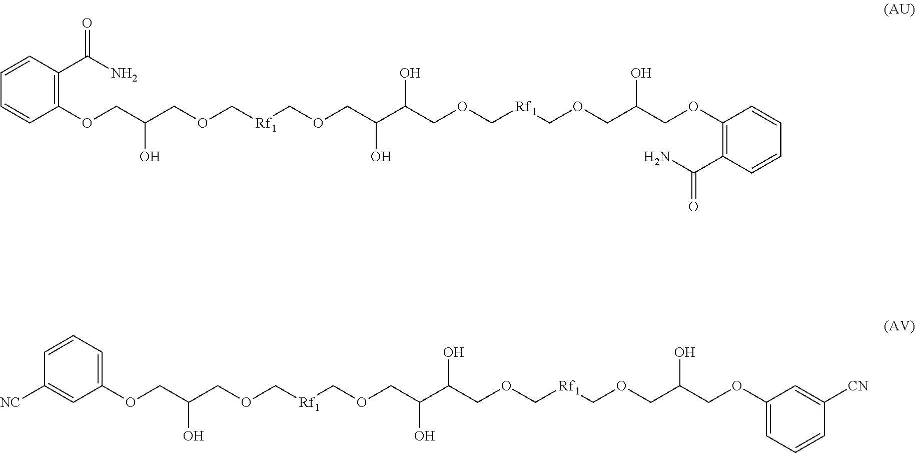

(in the two Rf₁'s in Formula (AU), h and i each indicate an average degree of polymerization, a represents 1 to 20, and i represents 0 to 20; and the average degrees of polymerization in the two Rf₁'s may be the same as or different from each other)
(in the two Rf₁'s in Formula (AV), h and i each indicate an average degree of polymerization, h represents 1 to 20; and i represents 0 to 20; and the average degrees of polymerization in the two Rf₁'s may be the same as or different from each other)

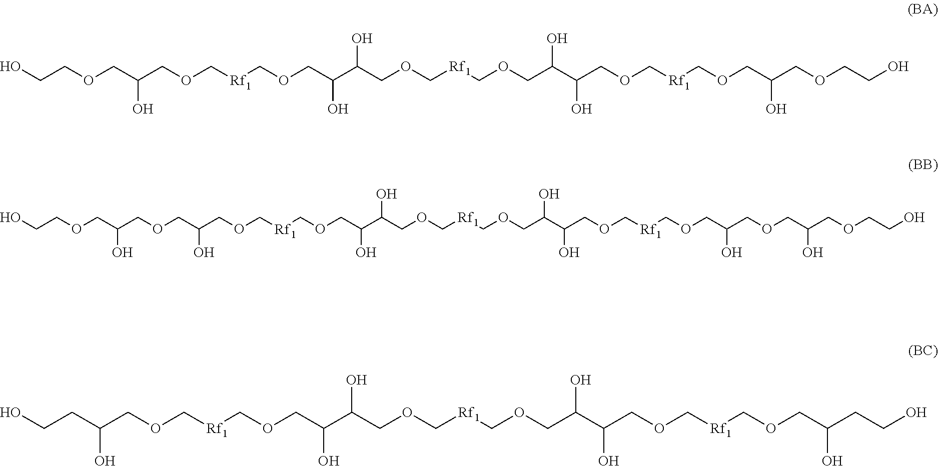

(in the three Rf₁'s in Formula (BA), h and i each indicate an average degree of polymerization, h represents 1 to 20, and i represents 0 to 20; and h and i in the three Rf₁'s may be different from each other or may be partially or entirely the same as each other)
(in the three Rf₁'s in Formula (BR), h and i each indicate an average degree of polymerization, h represents 1 to 20, and i represents 0 to 20; and h and i in the three Rf₁'s may be different from each other, or may be partially or entirely the same as each other)
(in the three Rf₁'s in Formula (BC), h and i each indicate an average degree of polymerization, h represents 1 to 20, and i represents 0 to 20; and h and i in the three Rf₁'s may be different from each other or may be partially or entirely the same as each other)

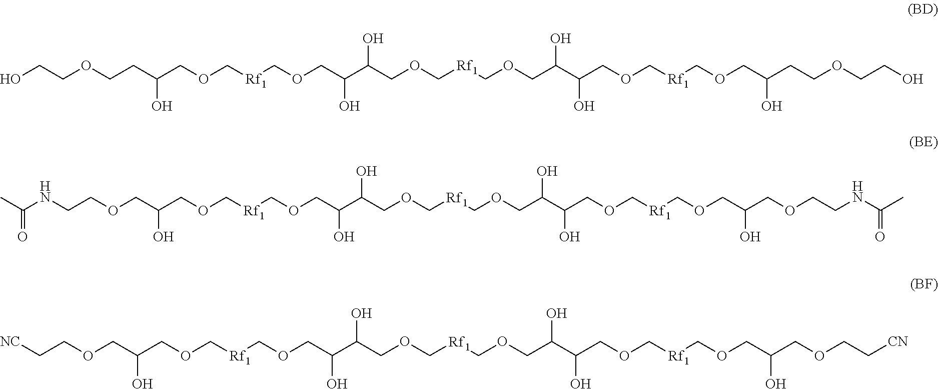

(in the three Rf₁'s in Formula (BD), h and i each indicate an average degree of polymerization, h represents 1 to 20, and i represents 0 to 20; and h and i in the three Rf₁'s may be different from each other or may be partially or entirely the same as each other)
(in the three Rf₁'s in Formula (BE), h and i each indicate an average degree of polymerization, h represents 1 to 20, and i represents 0 to 20; and h and i in the three Rf₁'s may be different from each other or may be partially or entirely the same as each other)
(in the three Rf₁'s in Formula (BF), h and i each indicate an average degree of polymerization, h represents 1 to 20, and i represents 0 to 20; and h and i in the three Rf₁'s may be different from each other, or may be partially or entirely the same as each other)

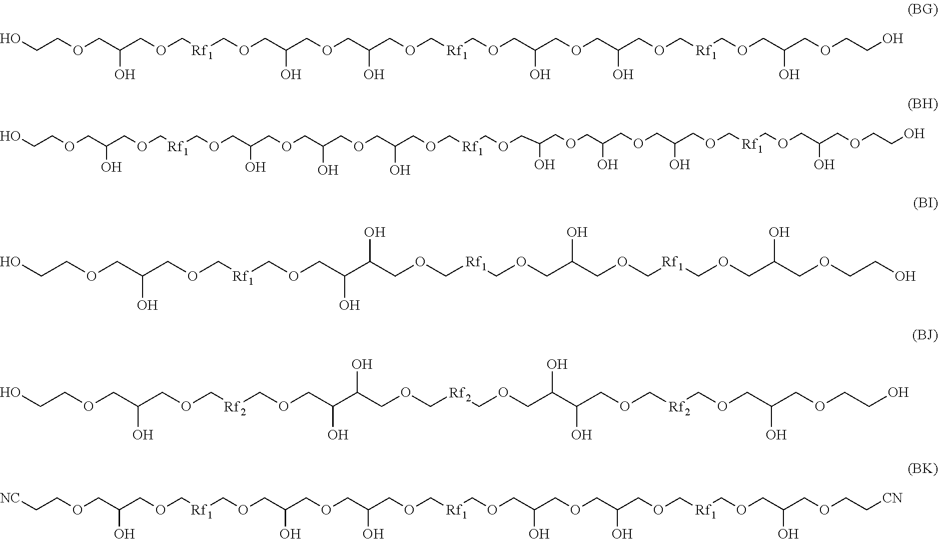

(in the three Rf₁'s in Formula (BG), h and i each indicate an average degree of polymerization, h represents 1 to 20, and i represents 0 to 20; and h and i in the three Rf₁'s may be different from each other or may be partially or entirely the same as each other)
(in the thrice Rf₁'s in Formula (BH), h and i each indicate an average degree of polymerization, h represents 1 to 20, and i represents 0 to 20; and h and i in the three Rf₁'s may be different from each other or may be partially or entirely the same as each other)
(in the three Rf₁'s in Formula (BT), h and i each indicate an average degree of polymerization, h represents 1 to 20, and i represents 0 to 20; and h and i in the three Rf₁'s may be different from each other or may be partially or entirely the same as each other)
(in the three Rf₂'s in Formula (BJ), j indicates an average degree of polymerization and represents 1 to 15; and j's in the three Rf₂'s may be different from each other, or may be partially or entirely the same as each other)
(in the three Rf₁'s in Formula (BK), h and i each indicate an average degree of polymerization, h represents 1 to 20, and i represents 0 to 20; and h and i in the three Rf₁'s may be different from each other or may be partially or entirely the same as each other)

A number-average molecular weight (Mn) of the fluorine-containing ether compound of the present embodiment is preferably in a range of 500 to 10,000, and particularly preferably in a range of 1,000 to 5,000. The number of the repeating units may be 2,000 to 4,500, 2.500 to 4,000, 3,000 to 3,500), or the like. In a case where the number-average molecular weight is 500 or more, the lubricating layer consisting of the lubricant including the fluorine-containing ether compound of the present embodiment has excellent heat resistance. The number-average molecular weight of the fluorine-containing ether compound is more preferably 1,000 or more. In addition, in a case where the number-average molecular weight is 10,000 or less, the viscosity of the fluorine-containing ether compound is appropriate, and a lubricating layer with a small film thickness can be easily formed by applying the lubricant including the fluorine-containing ether compound. The number-average molecular weight of the fluorine-containing ether compound is preferably 5,000 or less since a viscosity which is easy to handle is obtained in a case of being applied to the lubricant.

The number-average molecular weight (Mn) of the fluorine-containing ether compound is a value measured by ¹H-NMR and ¹⁹F-NMR using AVANCE III 400 manufactured by Bruker BioSpin GrmbH. Specifically, the number of repeating units of the PFPE chain is calculated from an integrated value measured by ¹⁹F-NMR to determine the number-average molecular weight. In the measurement of nuclear magnetic resonance (NMR), a sample is diluted with a solvent of hexafluorobenzene/d-acetone (4/1 v/v) and measured. A reference of the ¹⁹F-NMR chemical shift is a peak of hexafluorobenzene at −164.7 ppm and a reference of the ¹H-NMR chemical shift is a peak of acetone at 2.2 ppm.

The fluorine-containing ether compound of the present embodiment is preferably subjected to molecular weight fractionation by an appropriate method to have a polydispersity (ratio of a weight average molecular weight (Mw)/a number-average molecular weight (Mn)) of 1.3 or less.

In the present embodiment, a method for fractionating the molecular weight is not particularly limited, and for example, a molecular weight fractionation by a silica gel column chromatography method, a gel permeation chromatography (GPC) method, or the like, a molecular weight fractionation by a supercritical extraction method, or the like can be used.

### [Production Method]

A method for producing a fluorine-containing ether compound of the present embodiment is not particularly limited, and the fluorine-containing ether compound can be produced by a known production method in the related art. The fluorine-containing ether compound of the present embodiment can be produced, for example, by a production method shown below.

[First Production Method (Case where x is 1)]
(Case where R¹ and R⁴ Are Each Terminal Group Represented by Formula (3-1), (3-2), or (3-4), R¹ and R⁴ Are Same as Each Other, and Two R²'s are Same as Each Other)

A fluorine-based compound in which a hydroxymethyl group (—CH₂OH) is arranged at each of both terminals of a perfluoropolyether chain corresponding to R² in Formula (1) is prepared.

Next, the hydroxy group of the hydroxymethyl group arranged at one of terminals of the fluorine-based compound and an epoxy compound having a group corresponding to R¹ (=a group corresponding to R⁴) in Formula (1) are reacted with each other (first reaction). Thus, an intermediate compound 1-1 having a group corresponding to R¹ (=a group corresponding to R⁴) at one of terminals of the perfluoropolyether chain corresponding to R² is obtained.

As the epoxy compound having a group corresponding to R¹ (=a group corresponding to R⁴) in Formula (1), for example, compounds represented by Formulae (5-1) to (5-14) can be used. THP in Formulae (5-1) to (5-10) represents a tetrahydropyranyl group.

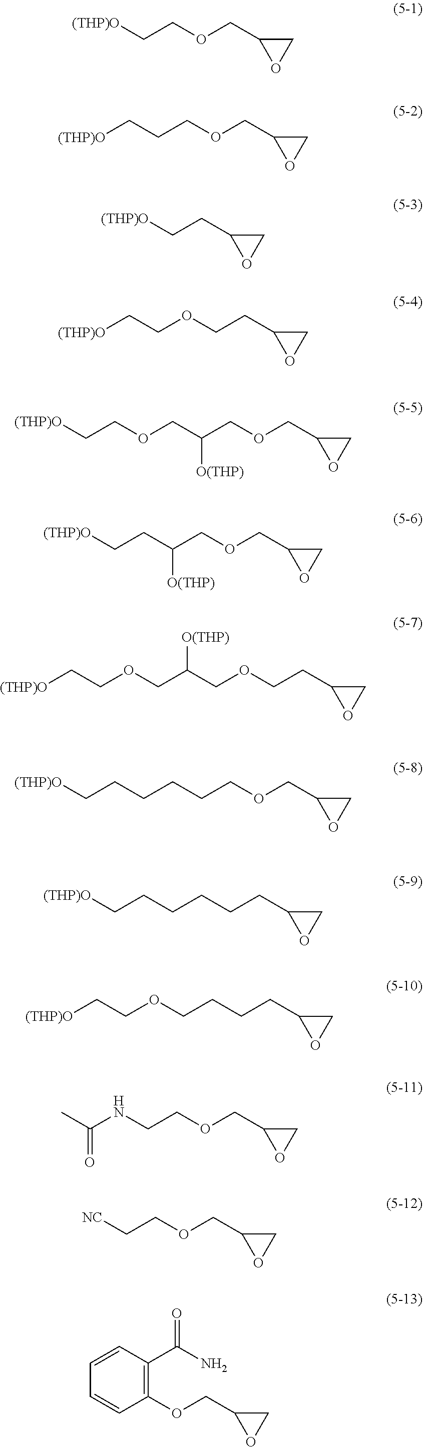

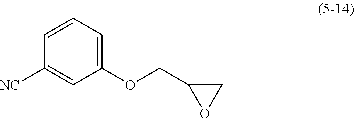 [SMILES: N#Cc1cccc(OCC2CO2)c1]

The epoxy compound having a group corresponding to R¹ (=a group corresponding to R⁴) in Formula (1) can be produced by, for example, a method shown below. That is, the epoxy compound can be produced by a method in which an alcohol having a structure (R in Formula (6-1)) corresponding to a part of the terminal group represented by R¹ (or R⁴) in Formula (1) and a halogen compound, such as a bromine compound and a chlorine compound, having an epoxy group (Formula (6-1) corresponds to a case where the halogen compound is the bromine compound) are reacted with each other, as shown in Formula (6-1). The number (a1 in Formula (6-1)) of methylene groups included in the halogen compound having an epoxy group is 1 to 4, and can be appropriately determined depending on the structure of the terminal group of a compound to be synthesized.

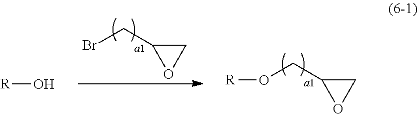

(in Formula (6-1), R represents a structure corresponding to a part of the terminal group represented by R¹ (or R⁴) in Formula (1)); and a1 represents an integer of 1 to 4)

In addition, the epoxy compound may be produced by a method shown below. That is, an alcohol having a structure (R in Formula (6-2)) corresponding to a part of the terminal group represented by R¹ (or R⁴) in Formula (1) and a halogen compound, such as a bromine compound and a chlorine compound, having an alkenyl group (Formula (6-2) corresponds to a case where the halogen compound is the bromine compound) are reacted with each other, as shown in Formula (6-2). Thereafter, a method in which the obtained compound is oxidized by reacting m-chloroperbenzoic acid (mCPBA) therewith can be used to produce the epoxy compound. The number (a2 in Formula (6-2)) of methylene groups included in the halogen compound having an alkenyl group is 1 to 4, and can be appropriately determined depending on the structure of the terminal group of a compound to be synthesized.

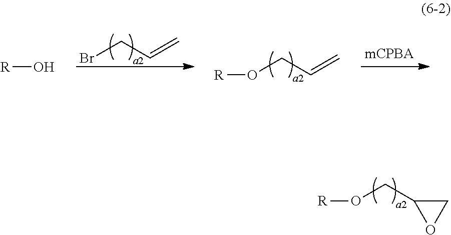

(in Formula (6-2), R represents a structure corresponding to a part of the terminal group represented by R¹ (or R⁴) in Formula (1); and a2 represents an integer of 1 to 4)

In addition, the epoxy compound may be produced by a method shown below. That is, an alcohol having a structure (R in Formula (6-3)) corresponding to a part of the terminal group represented by R¹ (or R⁴) in Formula (1), and a compound having an alkenyl group and an epoxy group are subjected to an addition reaction, as shown in Formula (6-3). Thereafter, a method in which the compound obtained by the addition reaction is oxidized with m-chloroperbenzoic acid (mCPBA) can be used to produce the epoxy compound. Before the compound obtained by the addition reaction is oxidized with m-chloroperbenzoic acid (mCPBA), a hydroxy group generated by the addition reaction may be protected with a known method. The number (a3 in Formula (6-3)) of methylene groups included in the compound having an alkenyl group and an epoxy group is 1 to 4, and can be appropriately determined depending on the structure of the terminal group of a compound to be synthesized.

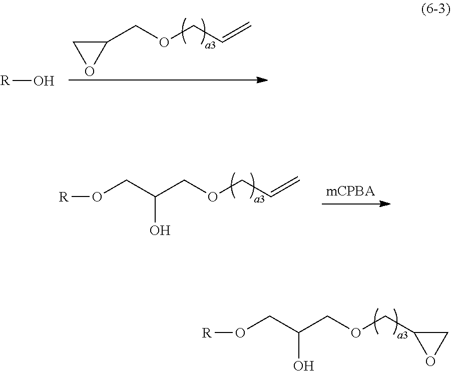

(in Formula (6-3), R represents a structure corresponding to a part of the terminal group represented by Rt (or R⁴) in Formula (1); and a3 represents an integer of 1 to 4)

There Aller, the hydroxy group at one of terminals of an intermediate compound 1-1 generated in the above-described first reaction and a compound having two epoxy groups corresponding to R³ in Formula (1) are reacted with each other (second reaction). As the compound having two epoxy groups corresponding to R³ in Formula (1), for example, compounds represented by Formulae (7-1) to (7-3) can be used. THP in Formula (7-3) represents a tetrahydropyranyl group.

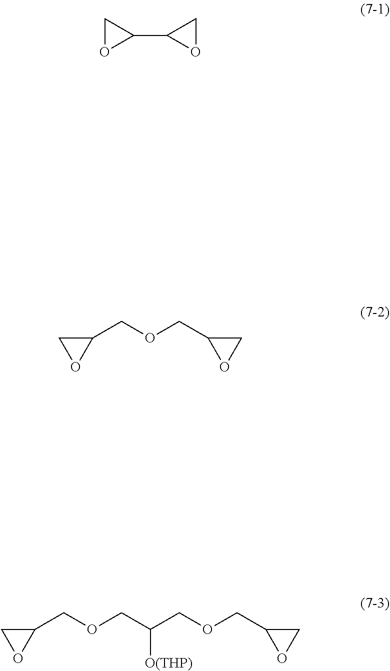

In a case where the compound represented by Formula (7-2) is used as the compound having two epoxy groups corresponding to R in Formula (1), the epoxy compound can be produced using, for example, a method shown below. That is, the epoxy compound can be produced using, a method in which an alkenyl group of a compound having an alkenyl group and an epoxy group (allyl glycidyl ether) is oxidized with m-chloroperbenzoic acid (mCPBA), as shown in Formula (8-1).

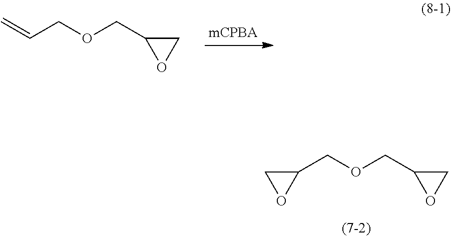

In a case where the compound represented by Formula (7-3) is used as the epoxy compound, the compound can be produced using, for example, a method shown below. That is, a halogen compound, such as a bromine compound and a chlorine compound, having an epoxy group (Formula (8-2) is a case where the halogen compound is the bromine compound) and an alcohol having an alkenyl group (allyl alcohol) are reacted with each other, as shown in Formula (8-2). Further, the compound can also be produced by a method in which a secondary hydroxy group contained in the compound generated after the reaction is protected using dihydropyran, and the alkenyl group is oxidized with n-chloroperbenzoic acid (mCPBA). THP in Formula (8-2) represents a tetrahydropyranyl group.

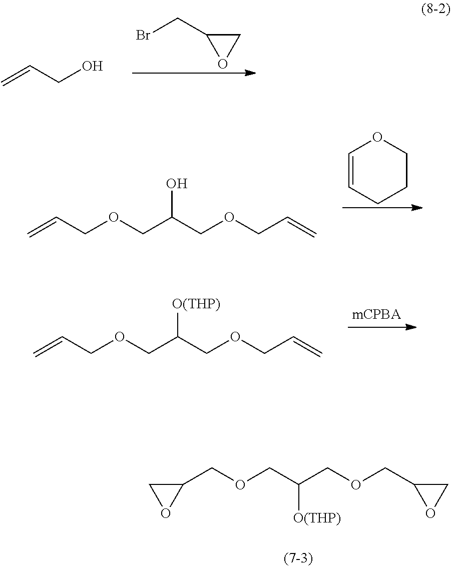

The compound represented by Formula (7-3) may be produced by, for example, the following method. That is, the compound can be produced using a method in which a compound having two hydroxy groups and a halogen compound, such as a bromine compound and a chlorine compound, having an epoxy group (Formula (8-3) is a case where the halogen compound is the bromine compound) are reacted with each other, as shown in Formula (8-3). THP in Formula (8-3) represents a tetrahydropyranyl group.

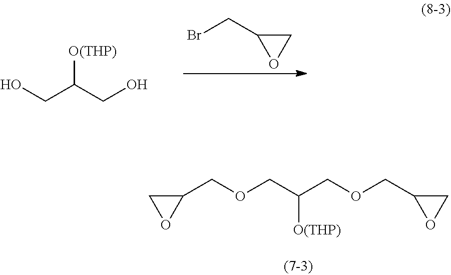

As the compound having two epoxy groups corresponding to R³ in Formula (1), a commercially available product may be used.

After the step above, a deprotection reaction can be performed by a known method to produce a compound in which x in Formula (1) is 1, R¹ and R⁴ are each a terminal group represented by Formula (3-1), (3-2), or (3-4), R¹ and R⁴ are the same as each other, and two R³'s are the same as each other.

[Second Production Method (Case where x is 1)]
(Case where R¹ and R⁴ Are Each Terminal Group Represented by Formula (3-3). R¹ and R⁴ Are Same as Each Other, and Two R²'s as Sante as Each Other)

A fluorine-based compound in which a hydroxymethyl group is arranged at each of both terminals of a perfluoropolyether chain corresponding to R in Formula (1) is prepared. Next, the hydroxy group of the hydroxymethyl group arranged at one of terminals of the fluorine-based compound and an epoxy compound having a structure corresponding to a chain-like portion (main chain portion) including Y of the terminal group represented by Formula (3-3) are reacted with each other first reaction). Thus, an intermediate compound 1-2, which has a group corresponding to the chain-like portion (main chain portion) including Y of the terminal group represented by Formula (3-3) and a secondary hydroxy group that forms a side chain portion of the terminal group represented by Formula (3-3) on one of terminals of the perfluoropolyether chain corresponding to R², is obtained.

As the epoxy compound having a structure corresponding to the chain-like portion (main chain portion) including Y of the terminal group represented by Formula (3-3) (=a group corresponding to R¹ and R⁴), for example, the compound represented by Formulae 5-1) to (5-4) and (5-9), a compound represented by Formula (17) which will be described later, or the like can be used.

Next, the hydroxy group at one of terminals of the intermediate compound 1-2 and a compound having a leaving group and a protected hydroxy group, which correspond to R³ in Formula (1), are reacted with each other to obtain an intermediate compound 1-3 (second reaction).

Examples of the compound having a leaving group and a protected hydroxy group, corresponding to R³, include a compound represented by Formula (14) which will be described later, a compound represented by Formula (31), and a compound represented by Formula (32). In Formula (31) and Formula (32), THP represents a tetrahydropyranyl group and Ts represents a tosyl group (p-toluenesulfonyl group).

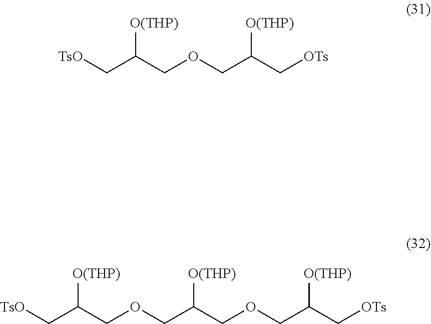

Next, a secondary hydroxy group that forms the side chain portion of the terminal group represented by Formula (3-3) contained in the intermediate compound 1-3 is reacted with a halogen compound having a protected hydroxy group corresponding to —(CH₂)ᵤ—OH in the side chain portion of the terminal group represented by Formula (3-3) (third reaction).

Examples of the halogen compound having a protected hydroxy group corresponding to —(CH₂)ᵤ—OH in the side chain portion of the terminal group represented by Formula (3-3) include a compound represented by Formula (16) which will be described later, a compound represented by Formula (33), and a compound represented by Formula (34). THP in Formula (33) and Formula (34) represents a tetrahydropyranyl group.

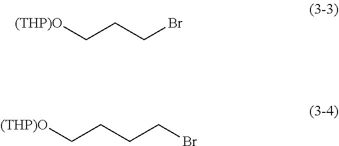

After the step above, a deprotection reaction can be performed by a known method to produce a compound in which x in Formula (1) is 1. R¹ and R⁴ are each a terminal group represented by Formula (3-3), R¹ and R⁴ are the same as each other, and two R³'s are the same av each other.

[Third Production Method (Case where x is 1)]
(Case where R¹ and R⁴ Are Bach Terminal Group Represented by Formula (3-1), (3-2), or (3-4), R¹ and R⁴ Are Different from Each Other, and/or Two R³'s are Different from Each Other)

First, the hydroxy group at one of terminals of a fluorine-based compound in which hydroxymethyl groups are each arranged at both terminals of a perfluoropolyether chain corresponding to R² on the R¹ side is reacted with an epoxy compound having a group corresponding to R¹ to obtain an intermediate compound 1a (first reaction).

Next, the hydroxy group at one of terminals of a fluorine-based compound in which hydroxymethyl groups are each arranged at both terminals of a perfluoropolyether chain corresponding to R² on the R⁴ side is reacted with an epoxy compound having a gr up corresponding to R¹ to obtain an intermediate compound 1b (second reaction).

Next, the hydroxy group at one of terminals of the intermediate compound 1a and a compound having an epoxy group and an alkenyl group, corresponding to R³ in Formula (1), are reacted with each other, and then a double bond contained in the purified compound is oxidized to obtain an intermediate compound 1-4 (third reaction).

As the compound having an epoxy group and an alkenyl group, corresponding to R¹ in Formula (1), for example, compounds represented by Formulae (9-1) to (9-3) which will be described later can be used.

Next, the hydroxy group at one of terminals of the intermediate compound 1b and the epoxy group of the intermediate compound 1.4 obtained in the third reaction are reacted with each other (fourth reaction).

After the step above, a deprotection reaction can be performed by a known method to produce a compound in which x in Formula (1) is 1. R¹ and R⁴ are each a terminal group represented by Formula (3-1), (3-2), or (3-4), R¹ and R⁴ are different from each other, and/or two R²'s are different from each other.

[Fourth Production Method (Case where x is 1)]
(Case where R¹ and R⁴ Are Each Terminal Group Represented by Formula (3-3), and R¹ and R⁴ are Different from Each Other) First, the hydroxy group at one of terminals of a fluorine-based compound in which hydroxymethyl groups are each arranged at both terminals of the perfluoropolyether chain corresponding to R² on the R¹ side and an epoxy compound having a structure corresponding to a chain-like portion (main chain portion) including Y of the terminal group represented by Formula (3-3) which is R¹ are reacted with each other to obtain an intermediate compound 1-2a (first reaction).

Next, the hydroxy group at one of terminals of a fluorine-based compound in which hydroxymethyl groups are each arranged at both terminals of the perfluoropolyether chain corresponding to R² on the R⁴ side and an epoxy compound having a structure corresponding to a chain-like portion (main chain portion) including Y of the terminal group represented by Formula (3-3) which is R¹ are reacted with each other to obtain an intermediate compound 1-2b (second reaction).

Next, the hydroxy group at one of terminals of the intermediate compound 1-2a and one leaving group of a compound having a leaving group and a protected hydroxy group, which correspond to R in Formula (1), are reacted with each other to obtain an intermediate compound 1-3a (third reaction).

Next, the hydroxy group at one of terminals of the intermediate compound 1.2b are reacted with each other and the leaving group of the intermediate compound 1-3a obtained in the third reaction are reacted with each other to obtain an intermediate compound 1-3b (fourth reaction).

Next, a secondary hydroxy group that forms the side chain portion of the terminal group represented by Formula (3-3) contained in the intermediate compound 1-3b is reacted with a halogen compound having a protected hydroxy group corresponding to —(CH₂)ᵤ—OH in the side chain portion of the terminal group represented by Formula (3-3) which is R¹ to obtain an intermediate compound 1-5 (fifth reaction).

Next, a secondary hydroxy group that forms the side chain portion of the terminal group represented by Formula (3-3) contained in the intermediate compound 1-5 is reacted with a halogen compound having a protected hydroxy group corresponding to —(CH₂)ᵤ—OH in the side chain portion of the terminal group represented by Formula (3-3) which is R⁴ (sixth reaction).

After the step above, a deprotection reaction can be performed by a known method to produce a compound in which x in Formula (1) is 1, R¹ and R⁴ are each a terminal group represented by Formula (3-3), and R¹ and R⁴ are different from each other.

[Fifth Production Method (Case where x is 1)]
(Case where R¹ is Terminal Group Represented by Formula (3-3) and R⁴ is Terminal Group Represented by Formula (3-1), (3-2), or (3-4))

First, an intermediate compound 1-3a is obtained in the same manner as in the fourth production method (first reaction).

Next, an intermediate compound 1b is obtained in the same manner as in the third production method (second reaction).

Next, the hydroxy group at one of terminals of the intermediate compound 1b and the leaving group of the intermediate compound 1-3a obtained in the first reaction are reacted with each other to obtain an intermediate compound 1-6 (third reaction).

Next, a secondary hydroxy group that forms the side chain portion of the terminal group represented by Formula (3-3) contained in the intermediate compound 1-6 is reacted with a halogen compound having a protected hydroxy group corresponding to —(CH₂)ᵤ—OH in the side chain portion of the terminal group represented by Formula (3-3) which is R¹ (fourth reaction).

After the above step, a deprotection reaction can be performed by a known method to produce a compound in which x in Formula (1) is 1, R¹ is the terminal group represented by Formula (3-3), and R³ is the terminal group represented by Formula (3-1). (3-2), or (3-4).

[Sixth Production Method (Case where x is 2)]
(Case where R¹ and R Are Each Terminal Group Represented by Formula (3-1), (3-2), or (3-4), R¹ and R⁴ Are Same as Each Other, Two R³'s are Same as Each Other, and R² on R¹ Side and R² on R⁴ Side Are Same as Each Other)

First, an intermediate compound 1-1 having a group corresponding to R¹ (=a group corresponding to R⁴) at one of terminals of the perfluoropolyether chain corresponding to R² on the R¹ side and the R⁴ side is produced in the same manner as in the first production method (first reaction).

Next, a fluorine-based compound in which a hydroxymethyl group (—CH₂OH) is arranged at each of both terminals of a perfluoropolyether chain corresponding to R¹ at the center of the molecule in Formula (I) is prepared. Next, the hydroxy group of the hydroxymethyl group arranged at both terminals of the fluorine-based compound and a compound having an epoxy group and an alkenyl group, corresponding to R¹ in Formula (1), are reacted with each other (second reaction).

As the compound having an epoxy group and an alkenyl group, corresponding to R³ in Formula (1), for example, the compounds represented by Formulae (9-1) to (9-3) can be used. THP in Formula (9-3) represents a tetrahydropyranyl group.

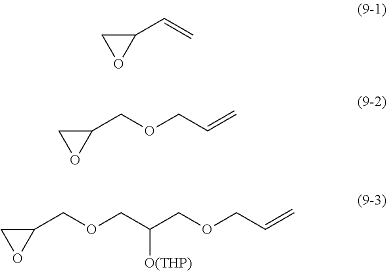

In a case where the compound represented by Formula (9-3) is used as the epoxy compound, the compound can be produced by, for example, a method shown below. That is, a halogen compound, such as a bromine compound and a chlorine compound, having an epoxy group (Formula (10-1) is a case where the halogen compound is the bromine compound) and an alcohol having an alkenyl group (allyl alcohol) are reacted with each other, as shown in Formula (10-1). Furthermore, the compound can also be produced by a method in which a secondary hydroxy group contained in the compound generated after the reaction is protected using dihydropyran, and then only one of the alkenyl groups is oxidized with m-chloroperbenzoic acid (mCPBA). Tip in Formula (10-1) represents a tetrahydropyranyl group.

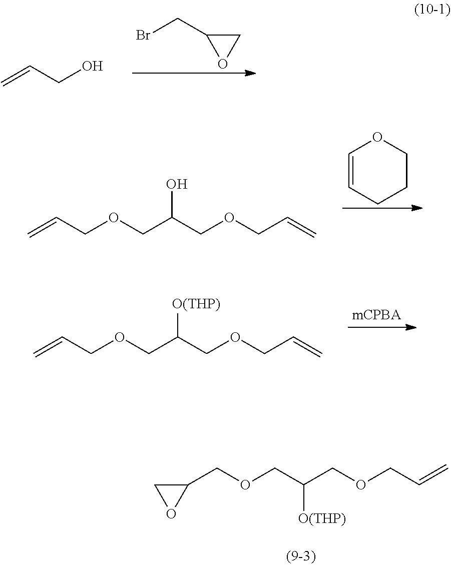

The compound represented by Formula (9-3) may be produced by, for example, the following method. That is, the compound can be produced using a method in which a compound having two hydroxy groups and a halogen compound, such as a bromine compound and a chlorine compound, having an alkenyl group (Formula (10-2) is a case where the halogen compound is the bromine compound) are reacted with each other, a substituent having an alkenyl group is introduced into one of the hydroxy groups, and then a halogen compound, such as a bromine compound and a chlorine compound, having an epoxy group (Formula (10-2) is a case where the halogen compound is the bromine compound) is reacted therewith, as shown in Formula (10-2). THP in Formula (10-2) represents a tetrahydropyranyl group.

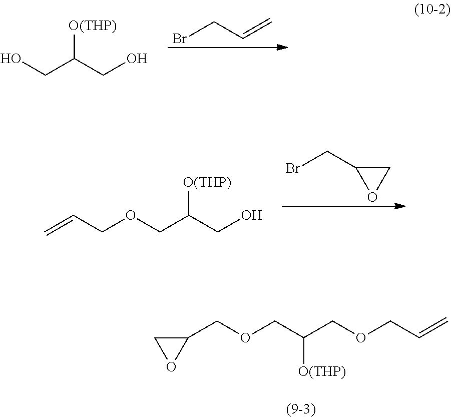

Next, the alkenyl group is oxidized by reacting m-chloroperbenzoic acid (mCPBA) with the compound generated after the second reaction (third reaction). Thus, an intermediate compound 2-1 having epoxy groups corresponding to two R³'s in Formula (1) at both terminals of the perfluoropolyether chain corresponding to R² at the center of the molecule in Formula (1) is obtained. The third reaction may be carried out after appropriately protecting the hydroxy group contained in the compound generated after the second reaction by a known method.

Thereafter, the hydroxy group of the hydroxymethyl group arranged at one of terminals of the intermediate compound 1-1 and epoxy groups arranged at both terminals of the intermediate compound 2-1 are reacted with each other (fourth reaction).

After the step above, a deprotection reaction can be performed by a known method to produce a compound in which x in Formula (1) is 2, R¹ and R⁴ are each a terminal group represented by Formula (3-1), (3-2), or (3-4), R¹ and R⁴ are the same as each other, two R³'s are the same as each other, and R² on the R¹ side and R² on the R⁴ side are the same as each other.

[Seventh Production Method (Case where x is 2)]
(Case where R¹ and R⁴ Are Each Terminal Group Represented by Formula (3-3), R¹ and R⁴ Are Same as Each Other, Two R³'s Are Same as Each Other, and R² on R¹ Side and R² on R¹ Side are Same as Each Other)

First, an intermediate compound 1-2, which has a group corresponding to the chain-like portion (main chain portion) including Y of the terminal group represented by Formula (3-3) and a secondary hydroxy group that forms a side chain portion of the terminal group represented by Formula (3-3) on one of terminals of the perfluoropolyether chain corresponding to R² on the R¹ side and the R⁴ side, is produced in the same manner as in the second production method (first reaction).

Next, a fluorine-based compound in which a hydroxymethyl group (—CH₂OH) is arranged at each of both terminals of a perfluoropolyether chain corresponding to R² at the center of the molecule in Formula (1) is prepared. Next, the hydroxy group of the hydroxymethyl group arranged at both terminals of the fluorine-based compound and a compound having a leaving group and a protected hydroxy group, corresponding to R³ in Formula (1), are reacted with each other (second reaction). Thus, an intermediate compound 2-2 having a structure and a leaving group corresponding to two R³'s in Formula (1) at both terminals of the perfluoropolyether chain corresponding to R¹ at the center of the molecule in Formula (1) is obtained.

Thereafter, the hydroxy group of the hydroxymethyl group arranged at one of terminals of the intermediate compound 1-2 and the intermediate compound 2-2 are reacted with each other to obtain an intermediate compound 2-3 (third reaction).

Next, a secondary hydroxy group that forms the side chain portion of the terminal group represented by Formula (3-3) contained in the intermediate compound 2-3 is reacted with a halogen compound having a protected hydroxy group corresponding to —(CH₂)ᵤ—OH in the side chain portion of the terminal group represented by Formula (3-3) (fourth reaction).

After the step above, a deprotection reaction can be performed by a known method to produce a compound in which x in Formula (1) is 2, R¹ and R⁴ are each a terminal group represented by Formula (3-3), R¹ and R⁴ are the same as each other, two R³'s are the same as each other, and R² on the R¹ side and R² on the R⁴ side are the same as each other.

[Eighth Production Method (Case where x is 2)]
(Case where Two R³'s Are Same as Each Other, R¹ and R⁴ Are Each Terminal Group Represented by Formula (3-1), (3-2), or (3-4), and R¹ and R⁴ Are Different from Each Other, and/or Case Where R t on R¹ Side and R on R⁴ Side are Different from Each Other)

First, an intermediate compound 1a having a group corresponding to R¹ at one of terminals of the perfluoropolyether chain corresponding to R on the R¹ side is produced in the same manner as in the third production method (first reaction). In addition, an intermediate compound 1b having a group corresponding to R⁴ at one of terminals of the perfluoropolyether chain corresponding to R² on the R⁴ side is produced in the same manner as in the third production method (second reaction).

Next, an intermediate compound 2-1 having epoxy groups corresponding to two R³'s in Formula (1) at both terminals of the perfluoropolyether chain corresponding to R¹ at the center of the molecule in Formula (1) is produced in the same manner as in the sixth production method (third reaction).

Thereafter, the hydroxy group of the hydroxymethyl group arranged at one of terminals of the intermediate compound 1a and an epoxy group arranged at one of terminals of the intermediate compound 2-1 are reacted with each other to obtain an intermediate compound 2-4 (fourth reaction).

Next, the hydroxy group of the hydroxymethyl group arranged at one of terminals of the intermediate compound 1b and the epoxy group of the intermediate compound 2-4 obtained in the fourth reaction are reacted with each other (fifth reaction).

After the step above, a deprotection reaction can be performed by a known method to produce a compound in a case where x in Formula (1) is 2, the two R³'s are the same as each other, R¹ and R⁴ are each a terminal group represented by Formula (3-1), (3-2), or (3-4), and R¹ and R⁴ are different from each other, and/or a case where R² on the R¹ side and R² on the R⁴ side are different from each other.

[Ninth Production Method (Case where x is 2)]
(Case where R¹ and R⁴ Are Each Terminal Group Represented by Formula (3-3), and R¹ and R⁴ are Different Front Each Other)

First, an intermediate compound 1-2a having a structure corresponding to a chain-like portion (main chain portion) including Y of the terminal group represented by Formula (3-3) which is R¹ at one of terminals of the perfluoropolyether chain corresponding to R² on the R¹ side is produced in the same manner as in the fourth production method (first reaction).

In addition, an intermediate compound 1-2b having a structure corresponding to a chain-like portion (main chain portion) including Y of the terminal group represented by Formula (3-3) which is R⁴ at one of terminals of the perfluoropolyether chain corresponding to R² on the R⁴ side is produced in the same manner as in the fourth production method (second reaction).

Next, an intermediate compound 2-2 having a structure and a leaving group corresponding to two R³'s in Formula (1) at both terminals of the perfluoropolyether chain corresponding to R² at the center of the molecule in Formula (1) is obtained in the same manner as in the seventh production method (third reaction).

Thereafter, the hydroxy group of the hydroxymethyl group arranged at one of terminals of the intermediate compound 1-2a and a leaving group arranged at one of terminals of the intermediate compound 2-2 are reacted with each other to obtain an intermediate compound 2-5 (fourth reaction).

Next, the hydroxy group of the hydroxymethyl group arranged at one of terminals of the intermediate compound 1-2b and the leaving group of the intermediate compound 2-5 obtained in the fourth reaction are reacted with each other to obtain an intermediate compound 2-6 fifth reaction).

Next, a secondary hydroxy group that forms the side chain portion of the terminal group represented by Formula (3-3) contained in the intermediate compound 2-6 is reacted with a halogen compound having a protected hydroxy group corresponding to —(CH₂)ᵤ—OH in the side chain portion of the terminal group represented by Formula (3-3) which is R¹ to obtain an intermediate compound 2-7 (sixth reaction).

Next, a secondary hydroxy group that forms the side chain portion of the terminal group represented by Formula (3-3) contained in the intermediate compound 2-7 is reacted with a halogen compound having a protected hydroxy group corresponding to —(CH₂)ᵤ—OH in the side chain portion of the terminal group represented by Formula (3-3) which is R⁴ (seventh reaction).

After the step above, a deprotection reaction can be performed by a known method to produce a compound in which x in Formula (1) is 2, R¹ and R⁴ are each a terminal group represented by Formula (3-3), and R¹ and R⁴ are different from each other.

[Tenth Production Method (Case where x is 2)]
(Case where R¹ is Terminal Group Represented by Formula (3-3) and R⁴ is Terminal Group Represented by Formula (3-1), (3.2), or (3-4))

First, an intermediate compound 2-5 is obtained in the same manner as in the ninth production method (first reaction).

In addition, an intermediate compound 1b having a group corresponding to R⁴ at one of terminals of the perfluoropolyether chain corresponding to R² on the R⁴ side is produced in the same manner as in the third production method (second reaction).

Thereafter, the hydroxy group of the hydroxymethyl group arranged at one of terminals of the intermediate compound 1b and the leaving group of the intermediate compound 2-5 are reacted with each other to obtain an intermediate compound 2-8 (third reaction).

Next, a secondary hydroxy group that forms the side chain portion of the terminal group represented by Formula (3-3) contained in the intermediate compound 2-8 is reacted with a halogen compound having a protected hydroxy group corresponding to —(CH₂)ᵤ—OH in the side chain portion of the terminal group represented by Formula (3-3) which is R¹ (fourth reaction).

After the above step, a deprotection reaction can be performed by a known method to produce a compound in which x in Formula (1) is 2, R¹ is the terminal group Represented by Formula (3-3), and R⁴ is the terminal group represented by Formula (3-1), (3-2), or (3-4).

[Eleventh Production Method (Case where x is 2)]
(Case where Two R³'s Are Different from Each Other, and at Least One thereof is Divalent Linking Group Represented by Formula (2-1) or (2-2), and R¹ and R⁴ are Each Terminal Group Represented by Formula (3-1), (3-2), or (3-4))

An intermediate compound 2-9 is produced using the following method, and in the sixth production method, the production can be performed by a method using the intermediate compound 2-9 instead of the intermediate compound 2-1.

That is, a fluorine-based compound in which a hydroxymethyl group (—CHO) is arranged at each of both terminals of a perfluoropolyether chain corresponding to R² at the center of the molecule in Formula (1) is prepared. Next, the hydroxy group of the hydroxymethyl group arranged at terminals of the fluorine-based compound and a compound having an epoxy group and an alkenyl group, corresponding to one of R³'s in Formula (1), are reacted with each other. Thereafter, the generated compound and a compound having an epoxy group and an alkenyl group corresponding to the other R¹ in Formula (1) are reacted with each other.

Thereafter, the alkenyl group is oxidized by reacting m-chloroperbenzoic acid (mCPBA) with the generated compound. Thus, an intermediate compound 2-9, which has an epoxy group corresponding to one of R³'s in Formula (1) at one end of the perfluoropolyether chain corresponding to R² at the center of the molecule in Formula (1), and has an epoxy group corresponding to the other R³ in Formula (1) at the other end part is obtained.

(Case where Two R³'s are Different from Each Other, and at Least One Thereof is Divalent Linking Group Represented by Formula (2-1) or (2-2), and R¹ and R⁴ are Each Terminal Group Represented by Formula (3-3))

An intermediate compound 2-10 is produced using the following method, and in the seventh production method, the production can be performed by a method using the intermediate compound 2-10 instead of the intermediate compound 2-2.

That is, a fluorine-based compound in which a hydroxymethyl group (—CH₂OH) is arranged at each of both terminals of a perfluoropolyether chain corresponding to R² at the center of the molecule in Formula (1) is prepared. Next, the hydroxy group of the hydroxymethyl group arranged at one terminal of the fluorine-based compound and a compound having a leaving group and a protected hydroxy group, corresponding to one of R³'s in Formula (1), are reacted with each other. Thereafter, the generated compound and a compound having the leaving group and the protected hydroxy group corresponding to the other R³ in Formula (1) are reacted with each other.

Thus, an intermediate compound 2-10, which has leaving group with a structure corresponding to one of R³'s in Formula (1) at one end of the perfluoropolyether chain corresponding to R² at the center of the molecule in Formula (I), and has a leaving group with a structure corresponding to the other R¹ in Formula (1) at the other end part is obtained.

### [Lubricant for Magnetic Recording Medium]

The lubricant for a magnetic recording medium of the present embodiment includes the fluorine-containing ether compound represented by Formula (1). In the lubricant of the present embodiment, known materials used as a material for the lubricant can be used in mixture as necessary as long as the characteristics are not impaired by incorporating the fluorine-containing ether compound represented by Formula (1).

Specific examples of the known materials include FOMBLIN (registered trademark) ZDIAC, FUMBLIN ZDEAL, and FOMBLIN AM-2001 (all of which are manufactured by Solvay Solexis.), and Moresco A20H (manufactured by Moresco).

The known material used in mixture with the lubricant of the present embodiment preferably has a number-average molecular weight of 1,000 to 10,000).

In a case where the lubricant of the present embodiment includes a material other than the fluorine-containing ether compound represented by Formula (1), a content of the fluorine-containing ether compound represented by Formula (1) in the lubricant of the present embodiment is preferably 50% by mass or more, more preferably 70% by mass or more, and still more preferably 90% by mass or more.

Since the lubricant of the present embodiment includes the fluorine-containing ether compound represented by Formula (1), it is possible to form a lubricating layer having excellent floating stability and a high pickup suppressing effect.

### [Magnetic Recording Medium]

The magnetic recording medium of the present embodiment is formed by sequentially providing at least a magnetic layer, a protective layer, and a lubricating layer on a substrate.

In the magnetic recording medium of the present embodiment, one or two or more underlayers can be provided between the substrate and the magnetic layer, as necessary. In addition, at least one of an adhesion layer and a soft magnetic layer can be provided between the underlayer and the substrate.

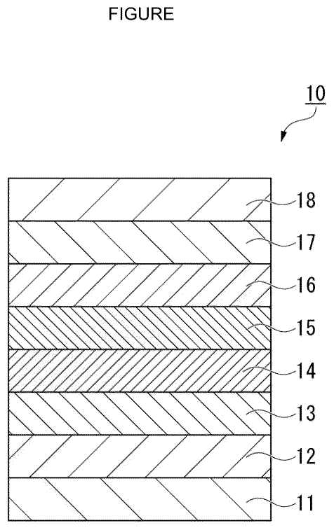

FIG. 1 is a schematic cross-sectional view showing an embodiment of the magnetic recording medium of the present invention.

The magnetic recording medium 10 of the present embodiment has a structure in which an adhesion layer 12, a soft magnetic layer 13, a first underlayer 14, a second underlayer 15, a magnetic layer 16, a protective layer 17, and a lubricating layer 18 are sequentially provided on a substrate 11.

### “Substrate”

As the substrate 11, for example, a non-magnetic substrate on which a film made of NiP or an NiP alloy is formed on a base made of a metal or an alloy material such as Al or an Al alloy can be used.

In addition, as the substrate 11, a non-magnetic substrate made of a non-metal material such as glass, ceramics, silicon, silicon carbide, carbon, or a resin may be used, or a non-magnetic substrate in which a film of NiP or an NiP alloy is formed on a base made of this non-metal material may be used.

### “Adhesion Layer”

The adhesion layer 12 prevents the progress of corrosion of the substrate 11, which occurs in a case where the substrate 11 and the soft magnetic layer 13 provided on the adhesion layer 12 are arranged in contact with each other.

A material for the adhesion layer 12 can be appropriately selected from, for example, Cr, a Cr alloy, Ti, a Ti alloy, CrTi, NiAl, and an AlRu alloy. The adhesion layer 12 can be formed by, for example, a sputtering method.

### “Soft Magnetic Layer”

The soft magnetic layer 13 preferably has a structure in which a first soft magnetic film, an intermediate layer consisting of an Ru film, and a second soft magnetic film are sequentially laminated. That is, the soft magnetic layer 13 preferably has a structure in which the intermediate layer made of an Ru film is interposed between two soft magnetic film layers to make the soft magnetic films above and below the intermediate layer be antiferromagnetically coupled (AFC).

Examples of materials for the first soft magnetic film and the second soft magnetic film include a CoZrTa alloy and a CoPe alloy.

Any of Zr, Ta, and Nb is preferably added to the CoFe alloy which is used in the first, soft magnetic film and the second soft magnetic film. This accelerates the amorphization of the first soft magnetic film and the second soft magnetic film. As a result, it is possible to improve the alignment of the first underlayer (seed layer) and to decrease the floating amount of a magnetic head.

The soft magnetic layer 13 can be formed by, for example, a sputtering method.

### “First Underlayer”

The first underlayer 14 is a layer that controls the alignment and the crystal size of the second underlayer 15 and the magnetic layer 16 provided thereon.

Examples of the first underlayer 14 include a Cr layer, a Ta layer, an Ru layer, a CrMo alloy layer, a CoW alloy layer, a CrW alloy layer, a CrV alloy layer, and a CrTi alloy layer.

The first underlayer 14 can be formed by, for example, a sputtering method.

### “Second Underlayer”

The second underlayer 15 is a layer that controls the alignment of the magnetic layer 16 to be good. The second underlayer 15 is preferably a layer consisting of Ru or an Ru alloy.

The second underlayer 15 may be a single layer or may be composed of a plurality of layers. In a case where the second underlayer 15 consists of a plurality of layers, all the layers may be composed of the same material, or at least one layer may be composed of a different material.

The second underlayer 15 can be formed by, for example, a sputtering method.

### “Magnetic Layer”

The magnetic layer 16 consists of a magnetic film in which a magnetization axis is oriented in a direction perpendicular or horizontal to the substrate surface. The magnetic layer 16 is a layer including Co and Pt. The magnetic layer 16 may be a layer including an oxide, Cr, B, Cu, Ta, Zr or the like in order to improve the SNR characteristic.

Examples of the oxide contained in the magnetic layer 16 include SiO₂, SiO, Cr₂O₃, CoO, Ta₂O₃, and TiO₂.

The magnetic layer 16 may be composed of one layer, or may be composed of a plurality of magnetic layers made of materials with different compositions.

For example, in a case where the magnetic layer 16 consists of three layers of a first magnetic layer, a second magnetic layer, and a third magnetic layer which are laminated in this order from the bottom, the first magnetic layer preferably has a granular structure made of a material containing Co, Cr, and Pt and further containing an oxide. As the oxide contained in the first magnetic layer, for example, an oxide of Cr, Si, Ta, Al, Ti, Mg, Co, or the like is preferably used. Among these, Ti, Cr₂O₃, SiO₂, or the like can be particularly suitably used. In addition, it is preferable that the first magnetic layer consists of a composite oxide obtained by adding two or more kinds of oxides. Among these, Cr₂O₃—SiO₂, Cr₂O₃—TiO₂, SiO₂—TiO₂, or the like can be particularly suitably used.

The first magnetic layer can include one or more kinds of elements selected from B, Ta, Mo, Cu, Nd, W, Nb, Sm, Th, Ru, and Re, in addition to Co, Cr, Pt, and oxides.

The same material as that for the first magnetic layer can be used for the second magnetic layer. The second magnetic layer preferably has a granular structure.

The third magnetic layer preferably has a non-granular structure consisting of a material including Co, Cr, and Pt and not including an oxide. The third magnetic layer can include one or more elements selected from B, Ta, Mo, Cu, Nd, W, Nb, Sm, Tb, Ru, Re, and Mn, addition to Co, Cr, and Pt.

In a case where the magnetic layer 16 is formed of a plurality of magnetic layers, it is preferable to provide a non-magnetic layer between the adjacent magnetic layers. In a case where the magnetic layer 16 consists of three layers of a first magnetic layer, a second magnetic layer, and a third magnetic layer, it is preferable to provide a non-magnetic layer between the first magnetic layer and the second magnetic layer and between the second magnetic layer and the third magnetic layer.

As the non-magnetic layer provided between the adjacent magnetic layers of the magnetic layer 16, for example, Ru, an Ru alloy, a CoCr alloy, or a CoCrX1 alloy (X1 represents one or two or more kinds of elements selected from Pt, Ta, Zr, Re, Ru, Cu, Nb, Ni, Mn, Ge, Si, O, N, W, Mo, Ti, V, and B) can be suitably used.

It is preferable that an alloy material including an oxide, a metal nitride, or a metal carbide is used in the non-magnetic layer provided between the adjacent magnetic layers of the magnetic layer 16. Specifically, as the oxide, for example, SiO₂, Al_{F}O₃, Ta₂O₅, Cr₂O₃, MgO, Y₂O₃, or TiO₂, can be used. As the metal nitride, for example, AlN, Si₃N₄, TaN, or rN can be used. As the metal carbide, for example, TaC, BC, or SiCe can be used.

The non-magnetic layer can be formed by, for example, a sputtering method.

The magnetic layer 16 is preferably a magnetic layer for perpendicular magnetic recording in which easy magnetization axis is directed to a direction perpendicular to the substrate surface in order to realize a higher recording density. The magnetic layer 16 may be a magnetic layer for in-plane magnetic recording.

The magnetic layer 16 may be formed by any of known methods in the related art, such as a deposition method, an ion beam sputtering method, and a magnetron sputtering method. The magnetic layer 16 is usually formed by a sputtering method.

### “Protective Layer”

The protective layer 17 protects the magnetic layer 16. The protective layer 17 may be composed of one layer or may be composed of a plurality of layers. As the protective layer 17, a carbon-based protective layer can be preferably used, and in particular, an amorphous carbon protective layer is preferable. In a case where the protective layer 17 is a carbon-based protective layer, the interaction with the polar group (in particular, the hydroxy group) included in the fluorine-containing ether compound in the lubricating layer 18 is further enhanced, which is thus preferable.

An adhesive force between the carbon-based protective layer and the lubricating layer 18 can be controlled by forming the carbon-based protective layer with hydrogenated carbon and/or nitrogenated carbon and adjusting the hydrogen content and/or the nitrogen content in the carbon-based protective layer. The hydrogen content in the carbon-based protective layer, as measured by hydrogen forward scattering (HFS), is preferably 3 atomic % to 20 atomic %. In addition, the nitrogen content in the carbon-based protective layer, measured by an X-ray photoelectron spectroscopy (XPS), is preferably 4 atonic % to 15 atomic %.

Hydrogen and/or nitrogen included in the carbon-based protective layer does not need to be evenly contained in the entire carbon-based protective layer. For example, the carbon-based protective layer is suitably formed as a composition gradient layer in which nitrogen is contained in the protective layer 17 on the lubricating layer 18 side and hydrogen is contained in the protective layer 17 on the magnetic layer 16 side. In this case, the adhesive force between the magnetic layer 16 and the carbon-based protective layer and between the lubricating layer 18 and the carbon-based protective layer is further improved.

A film thickness of the protective layer 17 is preferably 1 nm to 7 nm. In a case where the film thickness of the protective layer 17 is 1 nm or more, sufficient performance of the protective layer 17 can be obtained. The film thickness of the protective layer 17, which is 7 nm or less, is preferable from the viewpoint that the thickness of the protective layer 17 is reduced.

As a method for forming the protective layer 17, a sputtering method using a carbon-containing target material, a chemical vapor deposition (CVD) method using a hydrocarbon raw material such as ethylene and toluene, an ion beam deposition (OBD) method, or the like can be used.

In a case where the carbon-based protective layer is formed as the protective layer 17, the carbon-based protective layer can be formed, for example, by a DC magnetron sputtering method. In particular, in a case where the carbon-based protective layer is formed as the protective layer 17, it is preferable to form an amorphous carbon protective layer by a plasma CVD method. The amorphous carbon protective layer formed by the plasma CVD method has an even surface and a small roughness.

### “Lubricating Layer”

The lubricating layer 18 prevents contamination of the magnetic recording medium 10. In addition, the lubricating layer 18 reduce, a frictional force of a magnetic head of a magnetic recording and reproducing device which slides on the magnetic recording medium 10, thereby improving the durability of the magnetic recording medium 10.

As shown in FIG. 1, the lubricating layer 18 is formed on and in contact with the protective layer 17. The lubricating layer 18 is formed by applying the lubricant for a magnetic recording medium according to the above-described embodiment onto the protective layer 17. Therefore, the lubricating layer 18 includes the above-described fluorine-containing ether compound.

In a case where the protective layer 17 arranged below the lubricating layer 18 is a carbon-based protective layer, the lubricating layer 18 is particularly bonded to the protective layer 17 with a high bonding force. As a result, it is easy to obtain the magnetic recording medium 10 in which the surface of the protective layer 17 is coated at a high coating rate even in a case where the thickness of the lubricating layer 18 is small, and it is possible to effectively prevent the contamination of the surface of the magnetic recording medium 10.

An average film thickness of the lubricating layer 18 is preferably 0.5 nm (5 Å) to 2.0 nt (20 Å), and more preferably 0.5 nm (5 Å) to 1.2 nm (12 Å). In a case where the average film thickness of the lubricating layer 18 is 0.5 nm or more, the lubricating layer 18 is formed with an even film thickness without Conning an island shape or a mesh shape. Therefore, the surface of the protective layer 17 can be coated with the lubricating layer 18 at a high coating rate. In addition, in a case where the average film thickness of the lubricating layer 18 is 2.0 nm or less, it is possible to sufficiently reduce the thickness of the lubricating layer 18, and to sufficiently decrease the floating amount of a magnetic head.

### “Method for Forming Lubricating Layer”

Examples of a method for forming the lubricating layer 18 include a method in which a magnetic recording medium during production in which respective layers up to the protective layer 17 are formed on the substrate 11 is prepared, and a lubricating layer forming solution is applied onto the protective layer 17 and dried.

The lubricating layer forming solution can be obtained by dispersing and dissolving the lubricant for a magnetic recording medium of the above-described embodiment in a solvent as necessary, and setting the viscosity and concentration to be suitable for application methods.

Examples of the solvent used for the lubricating layer forming solution include a fluorine-based solvent such as VERTREL (registered trademark) XF (product name, manufactured by Mitsui DuPont Fluorochemicals Co., Ltd.).

A method for applying the lubricating layer forming solution is not particularly limited, and examples thereof include a spin coating method, a spraying method, a paper coating method, and a dipping method.

In a case where the dipping method is used, for example, the following method can be used. First, the substrate 11 on which each of layers up to the protective layer 17 is formed is immersed in the lubricating layer forming solution which has been put into an immersion vessel of a dip coater. Next, the substrate 11 is lifted from the immersion vessel at a predetermined speed. Thus, the lubricating layer forming solution is applied onto the surface of the protective layer 17 of the substrate 11.

By using the dipping method, the lubricating layer forming solution can be evenly applied onto the surface of the protective layer 17 and the lubricating layer 18 with an even film thickness can be formed on the protective layer 17.

In the present embodiment, it is preferable to perform a thermal treatment on the substrate 11 on which the lubricating layer 18 has been formed. By performing the thermal treatment, the adhesion between the lubricating layer 18 and the protective layer 17 is improved, and the adhesive force between the lubricating layer 18 and the protective layer 17 is improved.

The thermal treatment temperature is preferably 100° C. to 180° C., and more preferably 100° C. to 160° C. In a case where the thermal treatment temperature is 100° C. or higher, an effect of improving the adhesion between the lubricating layer 18 and the protective layer 17 can be sufficiently obtained. In addition, by setting the thermal treatment temperature to 180° C. or lower, it is possible to prevent thermal decomposition of the lubricating layer 18 due to the thermal treatment. The thermal treatment time can be appropriately adjusted depending on the thermal treatment temperature, and is preferably 10 minutes to 120 minutes.

In the present embodiment, in order to further improve the adhesion of the lubricating layer 18 to the protective layer 17, a treatment of irradiating the lubricating layer 18 with ultraviolet rays (UV) before or after the thermal treatment may be performed.

The magnetic recording medium 10 of the present embodiment is formed by sequentially providing at least the magnetic layer 16, the protective layer 17, and the lubricating layer 18 on the substrate 11. In the magnetic recording medium 10 of the present embodiment, the lubricating layer 18 including the above-described fluorine-containing ether compound is formed on and in contact with the protective layer 17. The lubricating layer 18 has good floating stability and a high pickup suppressing effect. Therefore, the magnetic recording medium 10 of the present embodiment has excellent reliability and durability. Thus, the magnetic recording medium 10 of the present embodiment can decrease the floating amount of a magnetic head (for example, 10 nm or less), and can be stably operated for a long period of time even in a severe environment due to the diversification of applications. Therefore, the magnetic recording medium 10 of the present embodiment is particularly suitable as a magnetic disk mounted in a magnetic disk device of a load unload (LUL) system.

### EXAMPLES

Hereinafter, the present invention will be described in more detail with reference to Examples and Comparative Examples. The present invention is not limited to only Examples described below.

### Example 1

The compound represented by Formula (AA) was obtained by a method shown below.

### (First Reaction)

20 g of a compound (number-average molecular weight: 1,000, molecular weight distribution: 1.1) represented by HOCH₂CF₂O(CF₂CF₂O)ₕ(CF₂O)ᵢCF₂CH₂OH (in the formula, h indicating an average degree of polymerization is 4.5 and i indicating an average degree of polymerization is 4.5), 4.12 g of the compound represented by Formula (5.1), and 20 mL of t-butanol were charged into a 100 ml eggplant flask in a nitrogen gas atmosphere, and stirred at room temperature until a homogenous mixture was obtained. 1.12 g of potassium tert-butoxide was added to the mixture and stirred at 70° C. for 16 hours to undergo a reaction.

The compound represented by Formula (5-1) was synthesized by protecting a hydroxy group of ethylene glycol monoallyl ether using dihydropyran, and then oxidizing the product with m-chloroperbenzoic acid.

The reaction product obtained after the reaction was cooled to 25° C., transferred to a separatory funnel into which 100 mL of water had been put, and extracted three times with 100 mL of ethyl acetate. The organic layer was washed with water and dehydrated over anhydrous sodium sulfate. After filtering the drying agent off, the filtrate was concentrated and the residue was purified by silica gel column chromatography to obtain 9.61 g of a compound represented by Formula (11) as the intermediate compound 1-1.

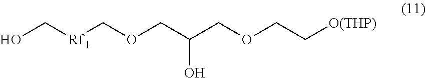

(in Formula (11), Rfⱼ is the PFPE chain represented by Formula (4-1); in Rf₁, h indicating an average degree of polymerization represents 4.5 and i indicating an average degree of polymerization represents 4.5; and THP represents a tetrahydropyranyl group)

### (Second Reaction)

Subsequently, 9.61 g of the compound represented by Formula (11), which is the intermediate compound 1-1 obtained above, 0.34 g of the compound represented by Formula (7-1), and 20 mL of t-butanol were charged into a 100 mL eggplant flask in a nitrogen gas atmosphere, and stirred at room temperature until a homogenous mixture was obtained. 0.45 g of potassium tert-butoxide was added to the mixture and stirred at 70° C. for 16 hours to undergo a reaction.

The reaction solution obtained after the reaction was cooled to room temperature, 50 g of a 10% hydrochloric acid/methanol solution (hydrochloric acid-methanol reagent (5% to 10%) manufactured by Tokyo Chemical Industry Co., Ltd.) was added thereto, and the mixture was stirred at room temperature for 4 hours. Thereafter, the reaction solution was gradually transferred to a separatory funnel into which 100 mL of a saturated aqueous sodium bicarbonate solution had been put, and then extracted twice with 200 mL of ethyl acetate. The organic layer was washed with 100 mL of saline, 100 mL of a saturated aqueous sodium bicarbonate solution, and 100 mL of saline in this order, and then dehydrated over anhydrous sodium sulfate. After filtering the drying agent off, the filtrate was concentrated and the residue was purified by silica gel column chromatography to obtain 2.81 g of a compound (AA) (in Formula (AA), Rf₁ is the PFPE chain represented by Formula (4-1), and in the two Rf₁'s, h indicating an average degree of polymerization represents 4.5 and i indicating an average degree of polymerization represents 4.5).

The obtained compound (AA) was subjected to ¹H-NMR measurement and ¹⁹F-NMR measurement, and a structure thereof was identified based on the following results.

¹H-NMR (acetone-D₆): δ [ppm]=3.40 to 3.85 (30H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 2

The compound represented by Formula (AB) was obtained by a method shown below.

The same operation as in Example 1, except that a compound represented by Formula (5-2) was used instead of the compound represented by Formula (5-1), was performed to obtain 2.92 g of the compound (AB) (in Formula (AB). Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-2) was synthesized by protecting one of hydroxy groups of 1,3-propanediol using dihydropyran, and then reacting the product with epibromohydrin.

The obtained compound (AB) was subjected to ¹H-NMR measurement and ¹⁹F-NMR measurement, and a structure thereof was identified based on the following results.

¹H-NMR (acetone-D₆): δ [ppm]=1.65 to 1.85 (4H), 3.40 to 3.85 (30H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 3

The compound represented by Formula (AC) was obtained by a method shown below.

The same operation as in Example 1, except that a compound represented by Formula (5.3) was used instead of the compound represented by Formula (5-1), was performed to obtain 2.54 g of the compound (AC) (in Formula (AC). Rf₁ is the PFPE chain represented by Formula (4.1), and in the two Rf₁'s, h indicating an average degree of polymerization represents 4.5 and i indicating an average degree of polymerization represents 4.5).

The compound represented by Formula (5-3) was synthesized by protecting a hydroxy group of 3-buten-1-ol using dihydropyran, and then oxidizing the product with m-chloroperhenzoic acid.

The obtained compound (AC) was subjected to ¹H-NMR measurement and ¹⁹F-NMR measurement, and the structure was identified based on the following results.

¹H-NMR (acetone-D₆): δ [ppm]=1.65 to 1.85 (4H), 3.40 to 3.85 (22H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 4

The compound represented by Formula (AD) was obtained by a method shown below.

The same operation as in Example 1, except that a compound represented by Formula (5-4) was used instead of the compound represented by Formula (5-1), was performed to obtain 2.54 g of the compound (AD) (in Formula (AD), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-4) was synthesized by reacting a hydroxy group of 3-buten-1-ol with 2-(2-bromoethoxy)tetrahydro-2H-pyran, and then oxidizing the product with m-chloroperbenzoic acid.

The obtained compound (AD) was subjected to ¹H-NMR measurement and ¹⁹F-NMR measurement, and a structure thereof was identified based on the following results.

¹H-NMR (acetone-D₆): δ [ppm]=1.65 to 1.85 (4H), 3.40 to 3.85 (30H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 5

The compound represented by Formula (AE) was obtained by a method shown below.

The same operation as in Example 1, except that a compound represented by Formula (5-5) was used instead of the compound represented by Formula (5-1), was performed to obtain 2.84 g of the compound (AF) (in Formula (AE), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-5) was synthesized by subjecting the compound represented by Formula (5-1) and an allyl alcohol to an addition reaction, protecting a hydroxy group of the obtained compound using dihydropyran, and further oxidizing the product with n-chloroperbenzoic acid.

The obtained compound (AE) was subjected to ¹H-NMR measurement and ¹⁹F-NMR measurement, and a structure thereof was identified based on the following results.

¹H-NMR (acetone-D₆): δ [ppM]=3.40 to 3.85 (42H), 3.85 to 4.10 (8H)

¹⁹F-NMR (acetone-D₆): δ [ppm]=−55.5 to −515 (18F), −78.5 (4F), −80.5 (4F), −91.0 to −88.5 (36F)

### Example 6

The compound represented by Formula (AF) was obtained by a method shown below.

The same operation as in Example 1, except that a compound represented by Formula (5-6) was used instead of the compound represented by Formula (5-1), was performed to obtain 2.84 g of the compound (AF) (in Formula (AF), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-6) was synthesized by subjecting the compound represented by Formula (5-3) and an allyl alcohol to an addition reaction, protecting a hydroxy group of the obtained compound using dihydropyran, and further oxidizing the product with m-chloroperbenzoic acid.

The obtained compound (AF) was subjected to ¹H-NMR measurement and ¹⁹F-NMR measurement, and a structure thereof was identified based on the following results.

¹H-NMR (acetone-D₆): δ [ppm]=1.65 to 1.85 (4H), 3.40 to 3.85 (34H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 7J

The compound represented by Formula (AG) was obtained by a method shown below.

The same operation as in Example 1, except that a compound represented by Formula (5-7) was used instead of the compound represented by Formula (5-1), was performed to obtain 2.84 g of the compound (AG) (in Formula (AG). Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-7) was synthesized by subjecting the compound represented by Formula (5-1) and 3-buten-1-ol to an addition reaction, protecting a hydroxy group of the obtained compound using dihydropyran, and further oxidizing the product with m-chloroperbenzoic acid.

The obtained compound (AG) was subjected to ¹H-NMR measurement and ¹⁹F-NMR measurement, and a structure thereof was identified based on the following results.

¹H-NMR (acetone-D₆): δ [ppm]=1.65 to 1.85 (4H), 3.40 to 3.85 (42H), 3.85 to 4.10 (81H)

¹⁹F-NMR (acetone-D₆): δ [ppm]=−55.5 to −51.5 (18F), −78.5 (4F), −80.5 (4F), −91.0 to −88.5 (36F)

### Example 8

The compound represented by Formula (Ali) was obtained by a method shown below.

### (First Reaction)

The compound represented by Formula (11) was obtained as the intermediate compound 1a in the same manner as in the first reaction or Example 1.

### (Second Reaction)

The same operation as the first reaction of Example 1, except that a compound represented by Formula (5-5) was used instead of the compound represented by Formula (5-1), was performed to obtain a compound represented by Formula (12) as the intermediate compound 1b.

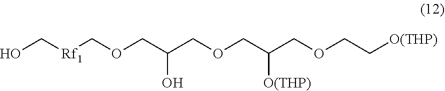

(in Formula (12), Rf₁ is the PFPE chain represented by Formula (4-1); in Rf₁, h indicating an average degree of polymerization represents 4.5, i indicating an average degree of polymerization represents 4.5; and THP represents a tetrahydropyranyl group)

### (Third Reaction)

Subsequently, 9.66 g of the compound represented by Formula (11), which is the intermediate compound 1a obtained above. 0.69 g of the compound represented by Formula (9-1), and 20 mL of t-butanol were charged into a 100 mL eggplant flask in a nitrogen gas atmosphere, and stirred at room temperature until a homogenous mixture was obtained. 0.45 g of potassium tert-butoxide was added to the mixture and stirred at 70° C. for 16 hours to undergo a reaction.

The reaction product obtained after the reaction was cooled to 25° C., transferred to a separatory funnel into which 100 mL of water had been put, and extracted three times with 100 mL of ethyl acetate. The organic layer was washed with water and dehydrated over anhydrous sodium sulfate. Ater filtering the drying agent off, the filtrate was concentrated and the residue was oxidized with m-chloroperbenzoic acid and then purified by silica gel column chromatography to obtain 6.48 g of a compound represented by Formula (13) as the intermediate compound 1-4.

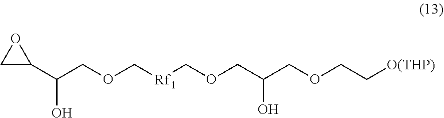

(in Formula (13), Rf₁ is the PFPE chain represented by Formula (4-1); in Rf₁, h indicating an average degree of polymerization represents 4.5 and i indicating an average degree of polymerization represents 4.5; and THP represents a tetrahydropyranyl group)

### (Fourth Reaction)

Subsequently, 7.21 g of the compound represented by Formula (12), which is the intermediate compound 1b obtained above, 6.48 g of the compound represented by Formula (13), which is the intermediate compound 1-4 obtained above, and 20 ml of t-butanol were charged into a 100 mL eggplant flask in a nitrogen gas atmosphere, and stirred at room temperature until a homogenous mixture was obtained. 0.45 g of potassium tert-butoxide was added to the mixture and stirred at 70° C. for 16 hours to undergo a reaction.

The reaction solution obtained after the reaction was cooled to room temperature, 50 g of a 10% hydrochloric acid/methanol solution (hydrochloric acid-methanol reagent (5% to 10%) manufactured by Tokyo Chemical Industry Co., Ltd.) was added thereto, and the mixture was stirred at room temperature for 4 hours. Thereafter, the reaction solution was gradually transferred to a separatory funnel into which 100 mL of a saturated aqueous sodium bicarbonate solution had been put, and then extracted twice with 200 mL of ethyl acetate. The organic layer was washed with 100 mL of saline, 100 mL of a saturated aqueous sodium bicarbonate solution, and 100 mL of saline in this order, and then dehydrated over anhydrous sodium sulfate. After filtering the drying agent off, the filtrate was concentrated and the residue was purified by silica gel column chromatography to obtain 4.34 g of a compound (AH) (in Formula (AH), Rf₁ is the PFPE chain represented by Formula (4-1), and in the two Rf₁'s, h indicating an average degree of polymerization represents 4.5 and i indicating an average degree of polymerization represents 4.5).

The obtained compound (AH) was subjected to ¹H-NMR measurement and ¹⁹F-NMR measurement, and a structure thereof was identified based on the following results.

¹H-NMR (acetone-d₆): δ [ppm]=3.40 to 3.85 (36H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 91

The compound represented by Formula (AI) was obtained by a method shown below.

The same operation as in Example 1, except that a compound represented by Formula (5-8) was used instead of the compound represented by Formula (5-1), was performed to obtain 3.06 g of the compound (AI) (in Formula (AI). Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-8) was synthesized by protecting one of hydroxy groups of 1,6-hexanediol using dihydropyran, and then reacting the product with epibromohydrin.

The obtained compound (AI) was subjected to ¹H-NMR measurement and ¹⁹F-NMR measurement, and a structure thereof was identified based on the following results.

¹H-NMR (acetone-D₆): δ [ppm]=1.25 to 1.85 (16H), 3.40 to 3.85 (301H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 10

The compound represented by Formula (AJ) was obtained by a method shown below.

The same operation as in Example 1, except that a compound represented by Formula (5.9) was used instead of the compound represented by Formula (5-1), was performed to obtain 2.81 g of the compound (AJ) (in Formula (A) Rf₁ is the PFPE chain represented by Formula (4.1), and in the two Rf₁'s. h indicating an average degree of polymerization represents 4.5 and i indicating an average degree of polymerization represents 4.5).

The compound represented by Formula (5-9) was synthesized by protecting a hydroxy group of 7-octen-1-ol using dihydropyran, and then oxidizing the product with m-chloroperhenzoic acid.

The obtained compound (AJ) was subjected to ¹H-NMR measurement and ¹⁹F-NMR measurement, and a structure thereof was identified based on the following results.

¹H-NMR (acetone-D₆): δ [ppm]=1.25 to 1.85 (16H), 3.40 to 3.85 (22H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 11

The compound represented by Formula (AK) was obtained by a method shown below.

The same operation as in Example 1, except that a compound represented by Formula (5-10) was used instead of the compound represented by Formula (5-1), was performed to obtain 2.61 g of the compound (AK) (in Formula (AK), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-10) was synthesized by protecting a hydroxy group of 2-bromoethanol with dihydropyran, further reacting 5-hexen-1-ol therewith, and then oxidizing the product with m-chloroperbenzoic acid.

The obtained compound (AK) was subjected to ¹H-NMR measurement and ¹⁹F-NMR measurement, and a structure thereof was identified based on the following results.

¹H-NMR (acetone-D₆): δ [ppm]=1.25 to 1.85 (12H), 3.40 to 3.85 (30H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 12

The compound represented by Formula (AL) was obtained by a method shown below.

The same operation as in Example 1, except that a compound represented by Formula (5-11) was used instead of the compound represented by Formula (5-1), was performed to obtain 2.42 g of the compound (AL) (in Formula (AL), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-11) was synthesized by reacting N-(2-hydroxyethyl)acetamide with epibromohydrin.

The obtained compound (AL) was subjected to ¹H-NMR measurement and ¹⁹F-NMR measurement, and a structure thereof was identified based on the following results.

¹H-NMR (acetone-D₆): δ [ppm]=1.90 to 2.05 (6H), 3.40 to 3.85 (28H), 3.85 to 4.10 (8H), 6.30 to 6.50 (2H)

¹⁹F-NMR (acetone-D₆): δ [ppm]=−55.5 to −51.5 (18F), −78.5 (4F), −80.5 (4F), 91.0 to −88.5 (36F)

### Example 13

The compound represented by Formula (AM) was obtained by a method shown below.

The same operation as in Example 1, except that a compound represented by Formula (5-12) was used instead of the compound represented by Formula (5-1), was performed to obtain 2.65 g of the compound (AM) (in Formula (AM), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5.12) was synthesized by reacting 2-cyanoethanol with epibromohydrin.

The obtained compound (AM) was subjected to ¹H-NMR measurement and ¹⁹F-NMR measurement, and a structure thereof was identified based on the following results.

¹H-NMR (acetone-D₆): δ [ppm]=2.00 to 2.10 (4H), 3.40 to 3.85 (24H), 3.85 to 4.10 (8H)

¹⁹F-NMR (acetone-D₆): δ [ppm]=−55.5 to −51.5 (18F), −78.5 (4F), −80.5 (41F), −91.0 to −88.5 (36F)

### Example 141

The compound represented by Formula (AN) was obtained by a method shown below.

The same operation as in Example 1, except that a compound represented by Formula (7-2) was used instead of the compound represented by Formula (7-1), was performed to obtain 2.41 g of the compound (AN) (in Formula (AN), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (7-2) was synthesized by oxidizing allyl glycidyl ether with m-chloroperbenzoic acid.

The obtained compound (AN) was subjected to ¹H-NMR measurement and ¹⁹F-NMR measurement, and a structure thereof was identified based on the following results.

¹H-NMR (acetone-D₆): δ [ppm]=3.40 to 3.85 (34H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 15

The compound represented by Formula (AO) was obtained by a method shown below.

The same operation as in Example 1, except that a compound represented by Formula (5-5) was used instead of the compound represented by Formula (5-1), and a compound represented by Formula (7-2) was used instead of the compound represented by Formula (7-1) was performed to obtain 2.41 g of the compound (AO) (in Formula (AO), Rf₁ is the PFPE chain represented by Formula (4-1), and in the two Rf₁'s, h indicating an average degree of polymerization represents 4.5 and i indicating an average degree of polymerization represents 4.5.

The obtained compound (AO) was subjected to ¹H-NMR measurement and ¹⁹F-NMR measurement, and a structure thereof was identified based on the following results.

¹H-NMR (acetone-D₆): δ [ppm]=3.40 to 3.85 (46H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 161

The compound represented by Formula (AP) was obtained by a method shown below.

The same operation as in Example 1, except that a compound represented by Formula (7-3) was used instead of the compound represented by Formula (7-1), was performed to obtain 2.56 g of the compound (AP) (in Formula (AP). Rf₁ is the PFPE chain represented by Formula (4-1), and in the two Rf₁'s, h indicating an average degree of polymerization represents 4.5 and i indicating an average degree of polymerization represents 4.5).

The compound represented by Formula (7-3) was synthesized by reacting two molecules of allyl alcohol with one molecule of epibromohydrin, protecting a secondary hydroxy group contained in the compound generated after the reaction using dihydropyran, and further oxidizing the product with m-chloroperbenzoic acid.

The obtained compound (AP) was subjected to ¹H-NMR measurement and ¹⁹F-NMR measurement, and a structure thereof was identified based on the following results.

¹H-NMR (acetone-D₆): δ [ppm]=3.40 to 3.85 (40H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 17

The compound represented by Formula (AQ) was obtained by a method shown below.

The same operation as in Example 1, except that a compound (number-average molecular weight: 1,000, molecular weight distribution: 1.1) represented by HOCF₂CF₂CF₂O(CF₂CF₂CF₂O)ⱼCF₂CF₂CH₂OH (in the formula, j indicating an average degree of polymerization is 4.5) was used instead of the compound represented by HOCH₂CF₂(CF₂CF₂O)ₕ(CF₂O)ᵢCF₂CH₂OH, was performed to obtain 2.74 g of the compound (AQ) (in Formula (AQ), Rf₂ is the PFPEi chain represented by Formula (4-2), and in Rf₂ indicating an average degree of polymerization represents 4.5).

The obtained compound (AQ) was subjected to ¹H-NMR measurement and ¹⁹F-NMR measurement, and a structure thereof was identified based on the following results.

¹H-NMR (acetone-D₆): δ [ppm]=3.40 to 3.85 (30H), 3.85 to 4.10 (8H)

¹⁹F-NMR (acetone-D₆): δ [ppm]=−84.0 to −83.0 (36F), −86.4 (8F), −124.3 (8F), −130.0 to −129.0 (18F)

### Example 18

The compound recemented by Formula (AR) was obtained by a method shown below.

The same operation as in Example 1, except that a compound (number-average molecular weight: 1,000, molecular weight distribution: 1.1) represented by HOCH₂CF₂CF₂CF₂O(CF₂CF₂CF₂CF₂O)ₖCF₂CF₂CF₂CH₂OH (in the formula, k indicating an average degree of polymerization was 3.0) was used instead of the compound represented by HOCF₂CF₂CF₂CF₂O(CF₂CF₂O)CF₂CH₂OH, was performed to obtain 2.67 g of the compound (AR) (in Formula (AR), Rf₃ is the PFPE chain represented by Formula (4-3), and in Rf₃, k indicating an average degree of polymerization represents 3.0).

The obtained compound (AR) was subjected to ¹H-NMR measurement and ¹⁹F-NMR measurement, and a structure thereof was identified based on the following results.

¹H-NMR (acetone-D₆): δ [ppm]=3.40 to 3.85 (30H), 3.85 to 4.10 (8H)

¹⁴F-NMR (acetone-D₆): δ [ppm]=−84.0 to −83.0 (32F), −122.5 (8F), −126.0 (24F), −129.0 to −128.0 (8F)

### Example 19

The compound represented by Formula (AS) was obtained by a method shown below.

### (First Reaction)

The compound represented by Formula (I1) was obtained as the intermediate compound 1-2 in the same manner as in the first reaction of Example 1.

### (Second Reaction)

Subsequently, 9.25 g of the compound represented by Formula (11) which is the intermediate compound 1-2 obtained above. 2.15 g of the compound represented by Formula (14), and 20 mL of dimethylformamide were charged into a 100 mL eggplant flask under a nitrogen atmosphere, and stirred at room temperature until a homogenous mixture was obtained. 2.88 g of cesium carbonate was added to the mixture and stirred at 70° C. for 16 hours to undergo a reaction.

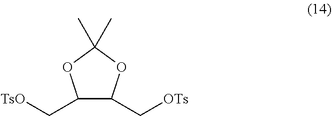 [SMILES: Cc1ccc(S(=O)(=O)OCC2OC(C)(C)OC2COS(=O)(=O)c2ccc(C)cc2)cc1]

(in Formula (14), Ts represents a tosyl group (p-toluenesulfonyl group)).

The reaction product obtained after the reaction was cooled to 25° C., transferred to a separatory funnel into which 100 mL of water had been put, and extracted three times with 100 mL of ethyl acetate. The organic layer was washed with water and dehydrated over anhydrous sodium sulfate. After filtering the drying agent off, the filtrate was concentrated, and the residue was oxidized with n-chloroperbenzoic acid and purified by silica gel column chromatography to obtain 7.22 g of a compound represented by Formula (15) as the intermediate compound 1-3.

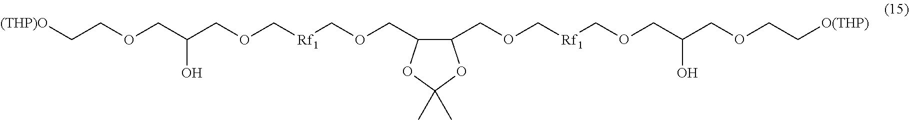

(in Formula (15), Rf₁ is the PFPE chain represented by Formula (4-1); in Rf₁, h indicating an average degree of polymerization represents 4.5 and i indicating an average degree of polymerization represents 4.5; and THP represents a tetrahydropyranyl group)

### (Third Reaction)

Subsequently, 7.21 g of the compound represented by Formula (15), which is the intermediate compound 1-3 obtained above, 1.51 g of a compound represented by Formula (16), and 20 mL of dimethylformamide were charged into a 100 mL eggplant flask in a nitrogen gas atmosphere, and stirred at room temperature until a homogenous mixture was obtained. 0.45 g of sodium hydride was added to the mixture and stirred at 40° C. for 16 hours to undergo a reaction.

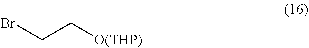

(in Formula (16), THP represents a tetrahydropyranyl group)

The reaction solution obtained after the reaction was cooled to room temperature, 50 g of a 10% hydrochloric acid/methanol solution (hydrochloric acid-methanol reagent (5% to 10%) manufactured by Tokyo Chemical Industry Co., Ltd.) was added thereto, and the mixture was stirred at room temperature for 4 hours. Thereafter, the reaction solution was gradually transferred to a separatory funnel into which 100 mL of a saturated aqueous sodium bicarbonate solution had been put, and then extracted twice with 200 mL of ethyl acetate. The organic layer was washed with 100 mL of saline, 100 mL of a saturated aqueous sodium bicarbonate solution, and 100 mL of saline in this order, and then dehydrated over anhydrous sodium sulfate. After filtering the drying agent off, the filtrate was concentrated and the residue was purified by silica gel column chromatography to obtain 3.51 g of a compound (AS) (in Formula (AS). Rf₁ is the PFPE chain represented by Formula (4-1), and in the two Rf₁s, h indicating an average degree of polymerization represents 4.5 and i indicating an average degree of polymerization represents 4.5).

The obtained compound (AS) was subjected to ¹H-NMR measurement and ¹⁹F-NMR measurement, and a structure thereof was identified based on the following results.

¹H-NMR (acetone-D₆): δ [ppm]=3.40 to 3.85 (38H), 3.85 to 4.10 (81H)

¹⁹F-NMR (acetone-D₆): δ [ppm]=−55.5 to −51.5 (18F), −78.5 (4F), −80.5 (4F), −91.0 to −88.5 (36F)

### Example 201

The compound represented by Formula (AT) was obtained by a method shown below.

The same operation as in Example 19, except that in the first reaction of Example 19, a compound represented by Formula (17) was used instead of the compound represented by Formula (5-1) to synthesize the intermediate compound 1-2, was performed to obtain 3.28 g of the compound (AT) (in Formula (AT). Rf₁ is the PFPE chain represented by Formula (4-1), and in the two Rf₁'s. h indicating an average degree of polymerization represents 4.5 and i indicating an average degree of polymerization represents 4.5).

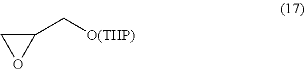

(in Formula (17), THP represents a tetrahydropyranyl group)

The compound represented by Formula (17) was synthesized by protecting a hydroxy group of allyl alcohol using dihydropyran, and then oxidizing the product with m-chloroperbenzoic acid.

The obtained compound (AT) was subjected to ¹H-NMR measurement and ¹⁹F-NMR measurement, and a structure thereof was identified based on the following results.

¹H-NMR (acetone-D₆); δ [ppm]=3.40 to 3.85 (30H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Example 21

The compound represented by Formula (AU) was obtained by a method shown below.

The same operation as in Example 1, except that a compound represented by Formula (5-13) was used instead of the compound represented by Formula (5-1) of Example 1, was performed to obtain 2.04 g of the compound (AU) (in Formula (AU), Rf₁ is the PFPE chain represented by Formula (4-1), and in the two Rtf's, h indicating an average degree of polymerization represents 4.5 and i indicating an average degree of polymerization represents 4.5).

The compound represented by Formula (5-13) was synthesized by reacting salicylamide with epibromohydrin.

The obtained compound (AU) was subjected to ¹H-NMR and ¹⁹F-NMR measurements, and a structure thereof was identified based on the following results.

¹H-NMR (acetone-D₆): δ [ppm]=3.40 to 3.85 (20H), 3.85 to 4.10 (8H), 6.70 to 8.20 (12H)

¹⁹F-NMR (acetone-D₆): δ [ppm]=−55.5 to −51.5 (18F), −78.5 (4F), −80.5 (4F), −91.0 to −88.5 (36F)

### Example 221

The compound represented by Formula (AV) was obtained by a method shown below.

The same operation as in Example 1, except that a compound represented by Formula (5-14) was used instead of the compound represented by Formula (5-1) of Example 1, was performed to obtain 2.21 g of the compound (AV) (in Formula (AV), Rf₁ is the PFPE chain represented by Formula (4-1), and in the two Rf₁'s, h indicating an average degree of polymerization represents 4.5 and i indicating an average degree of polymerization represents 4.5).

The compound represented by Formula (5-14) was synthesized by reacting 3-cyanophenol with epibromohydrin.

The obtained compound (AV) was subjected to ¹H-NMR and ¹⁹F-NMR measurements, and a structure thereof was identified based on the following results.

¹H-NMR (acetone-D₆): δ [ppm]=3.40 to 3.85 (20H), 3.85 to 4.10 (811). 7.20 to 7.60 (8H)

¹⁹F-NMR (aceone-D₆): δ [ppm]=−55.5 to −51.5 (18F), −78.5 (4F), −80.5 (4F), 91.0 to −88.5 (36F)

### Example 231

The compound represented by Formula (BA) was obtained by a method shown below.

The same operation as in Example 1, except that a compound represented by Formula (18) was used instead of the compound represented by Formula (7-1), was performed to obtain 3.61 g of the compound (BA) (in Formula (BA), Rf₁ is the PFPE chain represented by Formula (4-1), and in the three Rf₁'s, h indicating an average degree of polymerization represents 4.5 and i indicating an avenge degree of polymerization represents 4.5).

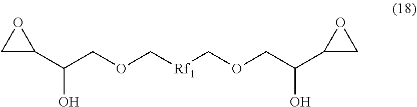

(in Formula (18), Rf₁ is the PFPE chain represented by Formula (4-1); and in Rf₁, h indicating an average degree of polymerization represents 4.5 and i indicating an average degree of polymerization represents 4.5)

The compound represented by Formula (18) was synthesized by reacting a compound (number-average molecular weight: 1,000, molecular weight distribution: 1.1) represented by HOCH₂CF₂O(CF₂CF₂O)ₕ(CF₂O)ᵢCF₂CH₂OH (in the formula, h indicating an average degree of polymerization is 4.5 and i indicating an average degree of polymerization is 4.5) with the compound represented by Formula (9-1), and then oxidizing the product with m-chloroperbenzoic acid.

The obtained compound (BA) was subjected to ¹H-NMR measurement and ¹⁹F-NMR measurement, and a structure thereof was identified based on the following results.

¹H-NMR (acetone-D₆): δ [ppm]=3.40 to 3.85 (38H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 241

The compound represented by Formula (BB) was obtained by a method shown below.

The same operation as in Example 23, except that a compound represented by Formula (5-5) was used instead of the compound represented by Formula (5-1), was performed to obtain 3.76 g of the compound (BB) (in Formula (BB), Rf₁ is the PFPE chain represented by Formula (4-1), and in the three Rf₁s, h indicating an average degree of polymerization represents 4.5 and i indicating an average degree of polymerization represents 4.5).

The obtained compound (BB) was subjected to ¹H-NMR measurement and ¹⁹F-NMR measurement, and a structure thereof was identified based on the following results

¹H-NMR (acetone-D₆): δ [ppm]=3.40 to 3.85 (50H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 25

The compound represented by Formula (BC) was obtained by a method shown below.

The same operation as in Example 23, except that a compound represented by Formula (5-3) was used instead of the compound represented by Formula (5-1), was performed to obtain 3.43 g of the compound (BC) (in Formula (BC), Rf₁ is the PFPE chain represented by Formula (4-1), and in the three Rf₃'s, h indicating an average degree of polymerization represents 4.5 and i indicating an average degree of polymerization represents 4.5).

The obtained compound (BC) was subjected to ¹H-NMR measurement and ¹⁹F-NMR measurement, and a structure thereof was identified based on the following results.

¹H-NMR (acetone-D₆): δ [ppm]=1.65 to 1.85 (4H), 3.40 to 3.85 (30H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 261

The compound represented by Formula (1D) was obtained by a method shown below.

The same operation as in Example 23, except that a compound represented by Formula (5-4) was used instead of the compound represented by Formula (5-1), was performed to obtain 3.43 g of the compound (HD) (in Formula (BD). Rf₁ is the PFPE chain represented by Formula (4-1), and in the three Rf₃'s, h indicating an average degree of polymerization represents 4.5 and i indicating an average degree of polymerization represents 4.5).

The obtained compound (BD) was subjected to ¹H-NMR measurement and ¹⁹F-NMR measurement, and a structure thereof was identified based on the following results.

¹H-NMR (acetone-D₆): δ [ppm]=1.65 to 1.85 (41H), 3.40 to 3.85 (42H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 27

The compound represented by Formula (BE) was obtained by a method shown below.

The same operation as in Example 23, except that a compound represented by Formula (5-11) was used instead of the compound represented by Formula (5-1), was performed to obtain 3.56 g of the compound (BE) (in Formula (BE), Rfₜ is the PFPE chain represented by Formula (4-1), and in the three Rf₁s, h indicating an average degree of polymerization represents 4.5 and i indicating an average degree of polymerization represents 4.5).

The obtained compound (BE) was subjected to ¹H-NMR measurement and ¹⁹F-NMR measurement, and a structure thereof was identified based on the following results.

¹H-NMR (acetone-D₆): δ [ppm]=1.90 to 2.05 (6H), 3.40 to 3.85 (36H), 3.85 to 4.10 (12H) 6.30 to 6.50 (2H)

¹⁹F-NMR (acetone-D₆): δ [ppm]=−55.5 to −51.5 (27F), −78.5 (6F), −80.5 (6), −91.0 to −88.5 (54F)

### Example 281

The compound represented by Formula (BF) was obtained by a method shown below.

The same operation as in Example 23, except that a compound represented by Formula (5-12) was used instead of the compound represented by Formula (5-1), was performed to obtain 3.82 g of the compound (BF) (in Formula (BF), Rf₁ is the PFPE chain represented by Formula (4-1), and in the three Rf₁, h indicating an average degree of polymerization represents 4.5 and i indicating an average degree of polymerization represents 4.5).

The obtained compound (BF) was subjected to ¹H-NMR measurement and ¹⁹F-NMR measurement, and a structure thereof was identified based on the following results.

¹H-NMR (acetone-D₆): δ [ppm]=2.00 to 2.10 (4H), 3.40 to 3.85 (32H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 291

The compound represented by Formula (BG) was obtained by a method shown below.

The same operation as in Example 23, except that a compound represented by Formula (9-2) was used instead of the compound represented by Formula (9-1), was performed to obtain 3.06 g of the compound (BG) (in Formula (BG), Rf₁ is the PFPE chain represented by Formula (4-1), and in the three Rf₁'s, h indicating an average degree of polymerization represents 4.5 and i indicating an average degree of polymerization represents 4.5).

The obtained compound (BG) was subjected to ¹H-NMR measurement and ¹⁹F-NMR measurement, and a structure thereof was identified based on the following results.

¹H-NMR (acetone-D₆): δ [ppm]=3.40 to 3.85 (461), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 30

The compound represented by Formula (BH) was obtained by a method shown below.

The same operation as in Example 23, except that a compound represented by Formula (9-3) was used instead of the compound represented by Formula (9-1), was performed to obtain 3.14 g of the compound (BH) (in Formula (BH), Rf₁ is the PEPE chain represented by Formula (4-1), and in the three Rf₁'s, h indicating an average degree of polymerization represents 4.5 and i indicating an average degree of polymerization represents 4.5).

The compound represented by Formula (9-3) was synthesized by reacting two molecules of allyl alcohol with one molecule of epibromohydrin, protecting a secondary hydroxy group contained in the generated compound using dihydropyran, and oxidizing one of carbon-carbon double bonds with m-chloroperbenzoic acid.

The obtained compound (BH) was subjected to ¹H-NMR measurement and ¹⁹F-NMR measurement, and a structure thereof was identified based on the following results.

¹H-NMR (acetone-D₆): δ [ppm]=3.40 to 3.85 (58H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 31

The compound represented by Formula (BI) was obtained by a method shown below.

The same operation as in Example 23, except that a compound represented by Formula (19) was used instead of the compound represented by Formula (18), was performed to obtain 3.24 g of the compound (31) (in Formula (I). Rf₁ is the PFPE chain represented by Formula (4-1), and in the three Rf₁'s, h indicating an average degree of polymerization represents 4.5 and i indicating an average degree of polymerisation represents 4.5).

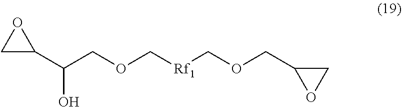

(in Formula (19), Rf₁ is the PFPE chain represented by Formula (4-1); and in Rf₁, h indicating an average degree of polymerization represents 4.5 and i indicating an average degree of polymerization represents 4.5)

The compound represented by Formula (19) was produced by a method shown below. 1,3-Butadiene monoepoxide was reacted with a compound (number-average molecular weight: 1,000, molecular weight distribution: 1.1) represented by HOCH₂CF₂O(CF₂CF₂O)ₕ(CF₂O)ᵢCF₂CH₂OH (in the formula, h indicating an average degree of polymerization is 4.5 and i indicating an average degree of polymerization is 4.5). Next, the generated compound was reacted with epibromohydrin. Thereafter, the carbon-carbon double bond was oxidized by reacting m-chloroperbenzoic acid with the compound generated to synthesize the compound to synthesize the compound represented by Formula (19).

The obtained compound (BI) was subjected to ¹H-NMR measurement and ¹⁹F-NMR measurement, and a structure thereof was identified based on the following results.

¹H-NMR (acetone-D₆): δ [ppm]=3.40 to 3.85 (36H), 3.85 to 4.10 (12H)

¹⁹F-NMR (acetone-D₆): δ [ppm]=−55.5 to −51.5 (27F), −78.5 (6F), −80.5 (6F), −91.0 to −88.5 (54F)

### Example 32

The compound represented by Formula (BJ) was obtained by a method shown below.

The same operation as in Example 23, except that a compound (number-average molecular weight: 1,000, molecular weight distribution: 1.1) represented by HOCH₂CF₂CF₂O(CF₂CF₂CF₂CF₂CF₂O)ⱼCF₂CF₂CH₂OH (in the formula, j indicating an average degree of polymerization is 4.5) was used instead of the compound represented by HOCH₂CF₂O(CF₂CF₂O)ₕ(CF₂)ᵢCF₂CH₂OH, was performed to obtain 2.74 g of the compound (BJ) (in Formula (BJ), Rf₂ is the PFPE chain represented by Formula (4-2), and in the three Rf₂ 's, j indicating an average degree of polymerization represents 4.5).

The obtained compound (BJ) was subjected to ¹H-NMR measurement and ¹⁹F-NMR measurement, and a structure thereof was identified based on the following results.

¹H-NMR (acetone-D₆): δ [ppm]=3.40 to 3.85 (38H), 3.85 to 4.10 (12H)

¹⁹F-NMR (acetone-D₆): δ [ppm]=−84.0 to −83.0 (54F), −86.4 (12F), −124.3 (12F), −130.0 to −129.0 (27F)

### Example 33

The compound represented by Formula (BK) was obtained by a method shown below.

The same operation as in Example 29, except that a compound represented by Formula (5-12) was used instead of the compound represented by Formula (5-1), was performed to obtain 3.21 g of the compound (BK) (in Formula (BK), Rf₁ is the PFPE chain represented by Formula (4-1), and in the three Rf₁'s, h indicating an average degree of polymerization represents 4.5 and i indicating an average degree of polymerization represents 4.5).

The obtained compound (BK) was subjected to ¹H-NMR measurement and ¹⁹F-NMR measurement, and a structure thereof was identified based on the following results.

¹H-NMR (acetone-D₆): δ [ppm]=2.00 to 2.10 (4H), 3.40 to 3.85 (40H), 3.85 to 4.10 (12H)

¹⁴F-NMR (acetone-D₆): δ [ppm]=−55.5 to −51.5 (27F), −78.5 (6F), −80.5 (6F), −91.0 to −88.5 (54F)

The values of x and the structures of R¹, R², R³, and R⁴ in a case where the compounds (AA) to (AV) and (BA) to (BK) of Examples 1 to 33 obtained as described above were each applied to Formula (1) are shown in Table 1. Furthermore, the structures of R¹ and R⁴ are the same in all compounds other than the compound (AH).

TABLE 1

| | | R¹ and R⁴ | | |
|---|---|---|---|---|
| | | (in case of (3-1), all are hydrogen atoms with | | |
| Compound | x | no description of A) | R² | R³ |
| (AA) | 1 | (3-1) p = 0/q = 0/r = 1 | (4-1) | (2-1) |
| (AB) | 1 | (3-1) p = 0/q = 0/r = 2 | (4-1) | (2-1) |
| (AC) | 1 | (3-2) s = 0/t = 1 | (4-1) | (2-1) |
| (AD) | 1 | (3-1) p = 1/q = 0/r = 1 | (4-1) | (2-1) |
| (AE) | 1 | (3-1) p = 0/q = 1/r = 1 | (4-1) | (2-1) |
| (AF) | 1 | (3-2) s = 1/t = 1 | (4-1) | (2-1) |
| (AG) | 1 | (3-1) p = 1/q = 1/r = 1 | (4-1) | (2-1) |
| (AH) | 1 | R¹: (3-1) p = 0/q = 0/r = 1 | (4-1) | (2-1) |
| | | R⁴: (3-1) p = 0/q = 1/r = 1 | | |
| (AI) | 1 | (3-1) p = 0/q = 0/r = 5 | (4-1) | (2-1) |
| (AJ) | 1 | (3-2) s = 0/t = 5 | (4-1) | (2-1) |
| (AK) | 1 | (3-1) p = 3/q = 0/r = 1 | (4-1) | (2-1) |
| (AL) | 1 | (3-1) p = 0/q = 0/r = 1/A = —NHCOCH₃ | (4-1) | (2-1) |
| (AM) | 1 | (3-1) p = 0/q = 0/r = 1/A = —CN | (4-1) | (2-1) |
| (AN) | 1 | (3-1) p = 0/q = 0/r = 1 | (4-1) | (2-2) 1 = 1 |
| (AO) | 1 | (3-1) p = 0/q = 1/r = 1 | (4-1) | (2-2) 1 = 1 |
| (AP) | 1 | (3-1) p = 0/q = 0/r = 1 | (4-1) | (2-2) 1 = 2 |
| (AQ) | 1 | (3-1) p = 0/q = 0/r = 1 | (4-2) | (2-1) |
| (AR) | 1 | (3-1) p = 0/q = 0/r = 1 | (4-3) | (2-1) |
| (AS) | 1 | (3-3) u = 2/Y = —CH₂OCH₂— | (4-1) | (2-1) |
| (AT) | 1 | (3-3) u = 2/Y = single bond | (4-1) | (2-1) |
| (AU) | 1 | (3-4) v = 1/B = —CONH₂ | (4-1) | (2-1) |
| (AV) | 1 | (3-4) v = 1/B = —CN | (4-1) | (2-1) |
| (BA) | 2 | (3-1) p = 0/q = 0/r = 1 | (4-1) | (2-1) |
| (BB) | 2 | (3-1) p = 0/q = 1/r = 1 | (4-1) | (2-1) |
| (BC) | 2 | (3-2) s = 0/t = 1 | (4-1) | (2-1) |
| (BD) | 2 | (3-1) p = 1/q = 0/r = 1 | (4-1) | (2-1) |
| (BE) | 2 | (3-1) p = 0/q = 0/r = 1/A = —NHCOCH₃ | (4-1) | (2-1) |
| (BF) | 2 | (3-1) p = 0/q = 0/r = 1/A = —CN | (4-1) | (2-1) |
| (BG) | 2 | (3-1) p = 0/q = 0/r = 1 | (4-1) | (2-2) 1 = 1 |
| (BH) | 2 | (3-1) p = 0/q = 0/r = 1 | (4-1) | (2-2) 1 = 2 |
| (BI) | 2 | (3-1) p = 0/q = 0/r = 1 | (4-1) | (2-1)/—OCH₂CH(OH)CH₂O— |
| (BJ) | 2 | (3-1) p = 0/q = 0/r = 1 | (4-2) | (2-1) |
| (BK) | 2 | (3-1) p = 0/q = 0/r = 1/A = —CN | (4-1) | (2-2) 1 = 1 |

### Comparative Example 1

A compound represented by Formula (ZA) was synthesized by the method described in Patent Document 1.

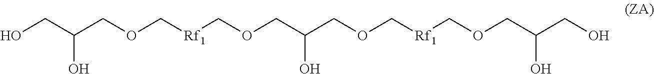

(in Formula (ZA), Rf₁ is the PFPE chain represented by Formula (4-1); and in the two Rf₁'s, h indicating an average degree of polymerization represents 7.0 and i indicating an average degree of polymerization represents 0)

### Comparative Example 2

A compound represented by Formula (ZB) was synthesized by the method described in Patent Document 2.

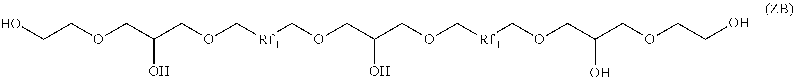

(in Formula (ZB), Rf₁ is the PFPE chain represented by Formula (4-1); and in the two Rf₁'s, h indicating an average degree of polymerization represents 4.5 and i indicating an average degree of polymerization represents 4.5)

### Comparative Example 3

A compound represented by Formula (ZC) was synthesized by the method described in Patent Document 3.

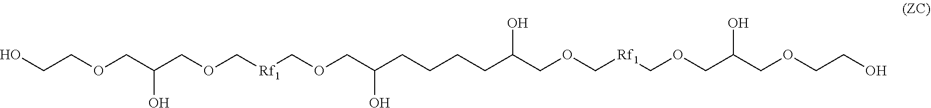

(in Formula (ZC), Rf₁ is the PFPE chain represented by Formula (4-1); and in the two Rf₁'s, h indicating an average degree of polymerization represents 4.5 and i indicating an average degree of polymerization represents 4.5)

### Comparative Example 4

A compound represented by Formula (ZD) was synthesized by the method described in Patent Document 4.

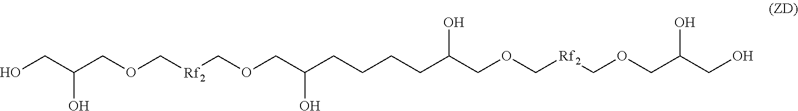

(in Formula (ZD), Rf₂ is the PFPE chain represented by Formula (4-2); and in the two Rf₂'s, j indicating an average degree of polymerization represents 4.5)

### Comparative Example 5

A compound represented by Formula (ZE) was synthesized by the method described in Patent Document 5.

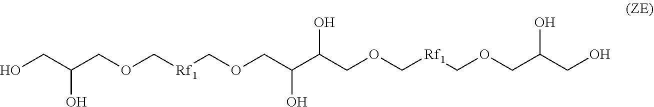

(in Formula (ZE), Rf₁ is the PFPE chain represented by Formula (4-1); and in the two Rf₁'s, h indicating an average degree of polymerization represents 4.5 and i indicating an average degree of polymerization represents 4.5)

### Compartive Example 6

The compound represented by Formula (ZF) was synthesized by a method shown below.

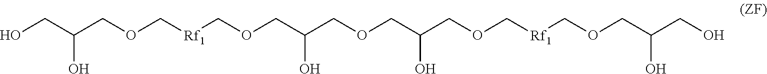

(in Formula (ZF), Rf₁ is the PFPE chain represented by Formula (4-1); and in the two Rf₁'s, h indicating an average degree of polymerization represents 4.5 and i indicating an average degree of polymerization represents 4.5)

The same operation as in Example 1, except that a compound represented by Formula (17) was used instead of the compound represented by Formula (5-1) and a compound represented by Formula (7-2) was used instead of the compound represented by Formula (7-1), was performed to obtain 2.21 g of the compound (ZF) (in Formula (ZF), Rf₁ is the PFPE chain represented by Formula (4-1), and in the two Rf₁'s, h indicating an average degree of polymerization represents 4.5 and i indicating an average degree of polymerization represents 4.5).

The obtained compound (ZF) was subjected to ¹H-NMR and ¹⁹F-NMR measurements, and a structure thereof was identified based on the following results.

¹H-NMR (acetone-D₆): δ [ppm]=3.40 to 3.85 (26H), 3.85 to 4.10 (8H)

¹⁹F-NMR (acetone-D₆): δ [ppm]=−55.5 to −51.5 (18F), −78.5 (4F), −80.5 (4F), −91.0 to −88.5 (36F)

### Compartive Example 7j

A compound represented by Formula (ZG) was synthesized by the method described in Patent Document 6.

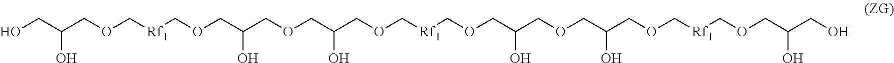

(in Formula (ZG), Rf₁ is the PFPE chain represented by Formula (4-1); and in the three Rf₁'s, h indicating an average degree of polymerization represents 7.0 and i indicating an average degree of polymerization represents 0)

### Compartive Example 8

A compound represented by Formula (ZH) was synthesized by the method described in Patent Document 7.

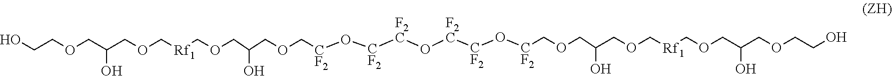

(in Formula (ZH), Rf₁ is the PFPE chain represented by Formula (4-1); and in the two Rf₁'s, h indicating an average degree of polymerization represents 4.5 and i indicating an average degree of polymerization represents 4.5)

### Compartive Example 9

A compound represented by Formula (ZI) was synthesized by the method described in Patent Document 8.

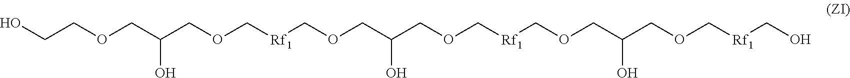

(in Formula (ZI), Rf₁ is the PFPE chain represented by Formula (4-1); and in the three Rf₁'s, h indicating an average degree of polymerization represents 4.5 and i indicating an average degree of polymerization represents 4.5)

The number-average molecular weight (Mn) of the compounds of Examples 1 to 33 and Comparative Examples 1 to 9 obtained as above were measured by the above-described method. The results thereof are shown in Tables 2 and 3.

Next, a lubricating layer forming solution was prepared using the compounds obtained in Examples 1 to 33 and Comparative Examples 1 to 9 by a method shown below. Then, a lubricating layer of a magnetic recording medium was formed by a method shown below using the obtained lubricating layer forming solution, thereby obtaining a magnetic recording medium of each of Examples 1 to 33 and Comparative Examples 1 to 9.

### “Lubricating Layer Forming Solution”

The compounds obtained in Examples 1 to 33 and Comparative Examples 1 to 9 were, each dissolved in a fluorine-based solvent, Vertrel (registered trademark) XF (product name, manufactured by Mitsui DuPont Fluorochemicals Co., Ltd.), and diluted with Vertrel XF such that a film thickness in a case of being applied onto the protective layer was 9.0 Å to 9.5 Å, thereby obtaining a lubricating layer forming solution.

### “Magnetic Recording Medium”

A magnetic recording medium in which an adhesion layer, a soft magnetic layer, a first underlayer, a second underlayer, a magnetic layer, and a protective layer had been sequentially provided on a substrate having a diameter of 65 mm was prepared. The protective layer was made of carbon.

The lubricating layer forming solutions of Examples 1 to 33 and Comparative Examples 1 to 9 were each applied on the protective layer of the magnetic recording medium, on which each of the layers up to the protective layer had been formed, by a dipping method. Furthermore, the dipping method was performed under the conditions of an immersion speed of 10 mm/sec, an immersion time of 30 sec, and a pulling-up speed of 1.2 mm/sec.

Thereafter, the magnetic recording medium on which the lubricating layer forming solution had been applied was placed in a thermostatic chamber and subjected to a thermal treatment at 120° C. for 10 minutes to remove the solvent in the lubricating layer forming solution and to improve the adhesion between the protective layer and the lubricating layer, thereby forming a lubricating layer on the protective layer to obtain a magnetic recording medium.

### (Measurement of Film Thickness)

The film thickness of the lubricating layer contained in the magnetic recording medium of Examples 1 to 33 and Comparative Examples 1 to 9 obtained as above was measured using a Fourier transform infrared spectrophotometer (FT-IR, product name: Nicolet iS50, manufactured by Thermo Fisher Scientific). The results thereof are shown in Tables 2 and 3.

Next, the magnetic recording medium of each of Examples 1 to 33 and Comparative Examples 1 to 9 was subjected to a floating stability test and a pickup characteristic test, each shown below.

### [Floating Stability Test]

The following glide test and credence measurement were performed, and the floating stability was evaluated based on the following evaluation criteria. The results thereof are shown in Tables 2 and 3.

### “Glide Test”

In the glide test, it is examined whether or not the magnetic recording medium has protrusions on the surface. That is, in a case where a floating amount (an interval between the magnetic recording medium and the magnetic head) is a height equal to or more than the height of the protrusion on the surface of the magnetic recording medium at a time of performing recording and reproduction on the magnetic recording medium using the magnetic head, the magnetic head may collide with the protrusion to to cause a damage to the magnetic head or a defect in the magnetic recording medium. In the glide test, the presence or absence of a protrusion having a height equal to or more than the floating amount of the front surface is examined for 50 magnetic recording media.

Specifically, an interval between the magnetic head for inspection and the magnetic recording medium was set to 0.25 microinches and the magnetic head for inspection was moved over the magnetic recording medium. In a case where a signal caused by the collision of the magnetic head for inspection with a protrusion on the surface of the magnetic recording medium was output from the magnetic head for inspection, the magnetic recording medium was determined to be a defective product, and otherwise, the magnetic recording medium was determined to be acceptable. Then, the evaluation was performed using the number of magnetic recording media determined to be acceptable among the 50 magnetic recording media.

### “Credence Measurement”

In a case where the glide test is performed, noise temporarily increases, and in a plurality of measurements at the same location on the magnetic recording medium, a signal caused by a collision with a protrusion on the surface may be detected or may not be detected. Such a phenomenon is referred to as a credence. The credence is not detected as a protrusion in the glide test, and is not used to determine to be acceptable or not in the glide test. However, a temporary increase in noise in the glide test generally indicates the unevenness of the lubricant layer or the presence of a relatively soft foreign matter. Thus, the glide test was performed on the magnetic recording medium, and the total number of times that the credence was detected was divided by the number of magnetic recording media (50 magnetic recording media) on which the glide test was performed to calculate an average value of the credences, which was used as an index indicating the smoothness and the cleanliness of the lubricant layer.

### “Evaluation Criteria”

A+: The number of the magnetic recording media that have passed the glide test is 45 or more and the average value of credences is less than 0.5.

A: The number of the magnetic recording media that have passed the glide test, is 45 or more and the average value of credences is 0.5 or more and less than 1.0.

B: The number of the magnetic recording media that have passed the glide test is or more and the average value of credences is 1.0 or more and less than 5.0.

C: The number of the magnetic recording media that have passed the glide test is or more and the average value of credences is 5.0 or more.

D: The number of the magnetic recording media that have passed the glide test is less than 45.

### [Pickup Characteristic Test]

The magnetic recording medium and the magnetic head were mounted on a spin stand, the magnetic recording medium was rotated at normal temperature under a reduced pressure (about 250 torr), and the magnetic head was allowed to float at a fixed point for 10 minutes. Thereafter, a surface, of the magnetic head facing the magnetic recording medium was analyzed using an electron spectroscopy for chemical analysis (ESCA) analyzer. An intensity (signal intensity (.au.)) of the fluorine-derived peak obtained by the analysis using the ESCA analyzer indicates the amount of the lubricant adhering to the magnetic head. The pickup characteristics were evaluated in accordance with an evaluation criteria shown below using the obtained signal intensity.

### “Evaluation Criteia”

A+: The signal intensity is less than 120 (almost no adhesion).

A: The signal intensity is 120 or more and less than 180 (the adherence amount was extremely small).

B: The signal intensity is 180 or more and less than 300 (the adherence amount was small).

C: The signal intensity is 300 or more and less than 100 (the adherence amount was large.

D: The signal intensity is 1,000 or more (the adherence amount is extremely large).

### [Comprehensive Evaluations]

From the results of the floating stability test and the pickup characteristic test, a comprehensive evaluation was performed based on the following criteria.

### [Comprehensive Evaluations]

A: Both the evaluation of the floating stability test and the evaluation of the pickup characteristic test were A+ or A.

B: One of the evaluation of the floating stability test and the evaluation of the pickup characteristic test was B, and the other was A+, A, or B.

C: One of the evaluation of the floating stability test and the evaluation of the pickup characteristic test was C, and the other was A+, A, B, or C.

D: At least one of the evaluation of the floating stability test and the evaluation of the pickup characteristic test was D.

TABLE 2

| | | | Film | Floating | Pickup | |
|---|---|---|---|---|---|---|
| | | Molecular | thickness | stability | characteristic | Comprehensive |
| | Compound | weight | (Å) | test | test | evaluation |
| Example 1 | (AA) | 2,325 | 9.1 | A | A | A |
| Example 2 | (AB) | 2,354 | 9.3 | A | A | A |
| Example 3 | (AC) | 2,261 | 9.4 | A | A | A |
| Example 4 | (AD) | 2,348 | 9.2 | A | A | A |
| Example 5 | (AE) | 2,478 | 9.1 | B | A+ | B |
| Example 6 | (AF) | 2,416 | 9.0 | B | A+ | B |
| Example 7 | (AG) | 2,489 | 9.2 | B | A+ | B |
| Example 8 | (AH) | 2,410 | 9.2 | B | A | B |
| Example 9 | (AI) | 2,431 | 9.3 | A | B | B |
| Example 10 | (AJ) | 2,385 | 9.1 | A | B | B |
| Example 11 | (AK) | 2,412 | 9.2 | A | B | B |
| Example 12 | (AL) | 2,403 | 9.3 | A | A+ | A |
| Example 13 | (AM) | 2,358 | 9.4 | A | A | A |
| Example 14 | (AN) | 2,381 | 9.3 | A | A | A |
| Example 15 | (AO) | 2,452 | 9.1 | B | A+ | B |
| Example 16 | (AP) | 2,458 | 9.1 | A+ | B | B |
| Example 17 | (AQ) | 2,331 | 9.2 | A | A | A |
| Example 18 | (AR) | 2,318 | 9.2 | A | A | A |
| Example 19 | (AS) | 2,415 | 9.1 | A | A | A |
| Example 20 | (AT) | 2,316 | 9.4 | A | A | A |
| Example 21 | (AU) | 2,518 | 9.1 | A | A+ | A |
| Example 22 | (AV) | 2,405 | 9.2 | A | A | A |
| Example 23 | (BA) | 3,421 | 9.3 | A | A | A |
| Example 24 | (BB) | 3,563 | 9.1 | B | A+ | B |
| Example 25 | (BC) | 3,352 | 9.0 | A | A | A |
| Example 26 | (BD) | 3,445 | 9.4 | A | A | A |
| Example 27 | (BE) | 3,506 | 9.2 | A | A+ | A |
| Example 28 | (BF) | 3,457 | 9.1 | A | A | A |
| Example 29 | (BG) | 3,502 | 9.5 | A | A | A |
| Example 30 | (BH) | 3,656 | 9.2 | A+ | B | B |
| Example 31 | (BI) | 3,365 | 9.4 | B | A | B |
| Example 32 | (BJ) | 3,433 | 9.3 | A | A | A |
| Example 33 | (BK) | 3,536 | 9.1 | A+ | A | A |

TABLE 3

| | | | Film | Floating | Pickup | |
|---|---|---|---|---|---|---|
| | | Molecular | thickness | stability | characteristic | Comprehensive |
| | Compound | weight | (Å) | test | test | evaluation |
| Comparative | (ZA) | 2,206 | 9.2 | D | D | D |
| Example 1 | | | | | | |
| Comparative | (ZB) | 2,278 | 9.4 | D | C | D |
| Example 2 | | | | | | |
| Comparative | (ZC) | 2,382 | 9.1 | D | C | D |
| Example 3 | | | | | | |
| Comparative | (ZD) | 2,292 | 9.4 | D | D | D |
| Example 4 | | | | | | |
| Comparative | (ZE) | 2,234 | 9.1 | C | D | D |
| Example 5 | | | | | | |
| Comparative | (ZF) | 2,281 | 9.3 | C | D | D |
| Example 6 | | | | | | |
| Comparative | (ZG) | 3,413 | 9.0 | C | D | D |
| Example 7 | | | | | | |
| Comparative | (ZH) | 2,755 | 9.2 | D | C | D |
| Example 8 | | | | | | |
| Comparative | (ZI) | 3,233 | 9.5 | D | D | D |
| Example 9 | | | | | | |

As shown in Table 2, the magnetic recording media of Examples 1 to 33 using the compounds (AA) to (AV) and (BA) to (BK) represented by Formula (1), in which R¹ and R⁴ each arranged at the terminals of the chain-like structure were each the terminal group represented by any of Formulae (3-1) to (3-4), and at least one of R³'s arranged between the adjacent perfluoropolyether chains was the divalent linking group represented by Formula (2-1) or (2-2), were evaluated as A+, A, or B in the floating stability test and the pickup characteristic test, and the comprehensive evaluations were all A or B. Thus, it was confirmed that the lubricating layers of the magnetic recording media of Examples 1 to 33 had good floating stability and a high pickup suppressing effect.

In the magnetic recording media of Examples 1 to 4, 12 to 14, 17 to 23, 25 to 29, 32, and 33, in which the compounds (AA) to (AD), (AL) to (AN), (AQ) to (AV), (BA), (BC) to (BG), (BJ), and (BK) were used, the evaluation of the floating stability test and the pickup characteristic test was A+ or A, and the comprehensive evaluation was A.

The number of the hydroxy groups included in x pieces of R³'s in the compounds (AA) to (AD), (AL) to (AN), (AQ) to (AT), (BA), (BC) to (BC), (BJ), and (BK) is 2, the number of the polar group, included in R¹ and R⁴ is 2, R¹ and R⁴ are represented by any of Formulae (3-1) to (3-3), and an alkylene chain in the main chain portion of Rt and R⁴ is not too long (the total of p and r in Formula (3-1) is 3 or less, t in Formula (3-2) is 4 or less, and the total number of carbon atoms and oxygen atoms contained in Y in Formula (3-3) is 3 or less). In addition, the number of the hydroxy groups included in R³ is 2, the number of the polar groups included in R¹ and R⁴ is 2, and R¹ and R⁴ are each represented by Formula (3-4) in the compounds (AU) and (AV).

In Examples 12, 21, and 27 using particularly the compounds (AL), (AU), and (BE) in which R was Formula (2-1) and Rt and R⁴ each include a group having an amide bond, among the compounds, the evaluation of the floating stability test was A and the evaluation of the pickup characteristic test was A+, which were excellent results.

In addition, in Examples 33 using the compound (BK) in which x was 2, R³ is Formula (2-2), l is 1, and R¹ and R⁴ each include a cyano acid, the evaluation of the evaluation of the floating stability test was A+ and the evaluation of the pickup characteristic test was A, which were excellent results.

In the compounds of Examples 12, 21, 27, and 33, it is presumed that a balance between a strength of the interaction between R¹ and R⁴ and the active point on the protective layer, and a strength of the interaction between R¹ and the active point on the protective layer was appropriate.

On the other hand, in Comparative Examples 1 to 9, in which the compounds (ZA) to (ZI) were used, both of the evaluations of the floating stability test and the pickup characteristic test were C or D, and the comprehensive evaluation was D.

More specifically, in Comparative Examples 1, 2, 8, and 9, in which the compounds (ZA), (ZB), (ZH), and (ZI) where the linking group arranged between the adjacent perfluoropolyether chains includes only one hydroxy group were used, the result of the floating stability test was D. This is considered to be because the number of the hydroxy groups in the linking group is insufficient, and thus, a sufficient interaction between the linking group and the protective layer is not obtained, the central portion of the fluorine-containing ether compound is floated, and thus, the smoothness of the lubricating layer is lost.

In addition, in Comparative Examples 3 and 4, in which the compound (ZC) and the compound (ZD) where the linking group arranged between the adjacent perfluoropolyether chains has two hydroxy groups, but a rigid alkylene chain to which four methylene groups were bonded was arranged between carbon atoms to which hydroxy groups were bonded was used, the result of the floating stability test was D. This is presumed to be because the motion of the hydroxy group contained in the linking group is restricted by the rigid alkylene chain included in the linking group. As a result, it is presumed that a sufficient interaction between the linking group portion of the compound and the protective layer is not obtained, the central portion of the compound is floated, and thus, the smoothness of the lubricating layer is lost.

In addition, in Comparative Examples 1 and 4 to 7, in which the compounds (ZA) and (ZD) to (ZG) having a terminal group in which carbon atoms to which hydroxy groups are bonded are directly bonded to each other were used, the evaluation of the pickup characteristic test was D. In these compounds, since the hydroxy groups included in the terminal groups are in close proximity to each other, the intramolecular interaction between the hydroxy groups in close proximity to each other occurs preferentially to the interaction between the hydroxy group and the protective layer. Therefore, it is considered that a sufficient interaction between the terminal group and the protective layer is not obtained, and the terminal portion of the compound is likely to float up and be adsorbed to the magnetic head.

In addition, in Comparative Example 9, in which the compound (ZI) including only one hydroxy group in one of terminal groups was used, the evaluation of the pickup characteristic test was D. This is presumed to be because the number of the hydroxy groups in the terminal group was insufficient, and thus, a sufficient interaction between the terminal group and the protective layer could not be obtained. As a result, it is presumed that the terminal portion of the compound is floated and easily adsorbed to the magnetic head.

### INDUSTRIAL APPLICABILITY

By using the lubricant for a magnetic recording medium including the fluorine-containing ether compound of the present invention, it is possible to form a lubricating layer having good floating stability and a high pickup suppressing effect.

### REFERENCE SIGNS LIST

10: magnetic recording medium
11: substrate
12: adhesion layer
13: soft magnetic layer
14: first underlayer
15: second underlayer
16: magnetic layer
17: protective layer
18: lubricating layer

1. A fluorine-containing ether compound represented by Formula (1), 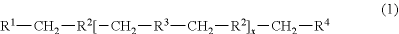
(in Formula (1), x represents an integer of 1 or 2; R² is a perfluoropolyether chain; (x+1) pieces of R²'s may be partially or entirely the same as or different from each other; R³ is a divalent linking group having a hydroxy group; at least one of x pieces of R³'s is a divalent linking group represented by Formula (2-1) or (2-2); in a case where x is 2, two R³'s may be the same as or different from each other; R¹ and R⁴ are each a terminal group having two to four polar groups, and has an oxygen atom bonded to a methylene group to which R² is bonded, a number of carbon atoms in a shortest distance between carbon atoms to which adjacent polar groups being 1 to 9 in R¹ and R⁴; and R¹ and R⁴ may be the same as or different from each other) 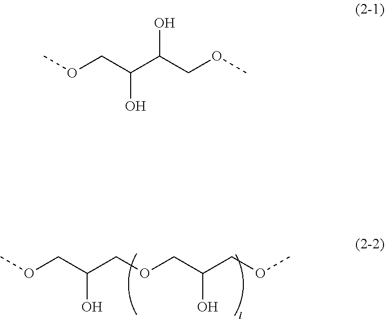
(in Formula (2-2), 1 represents an integer of 1 or 2).

2. The fluorine-containing ether compound according to claim 1,
wherein R¹ and R⁴ in Formula (1) each independently includes at least one polar group selected from the group consisting of a hydroxy group, a cyano group, and a group having an amide bond.

3. The fluorine-containing ether compound according to claim 1,
wherein R¹ and R⁴ in Formula (1) are each independently a terminal group represented by any of Formulae (3-1) to (3-4), 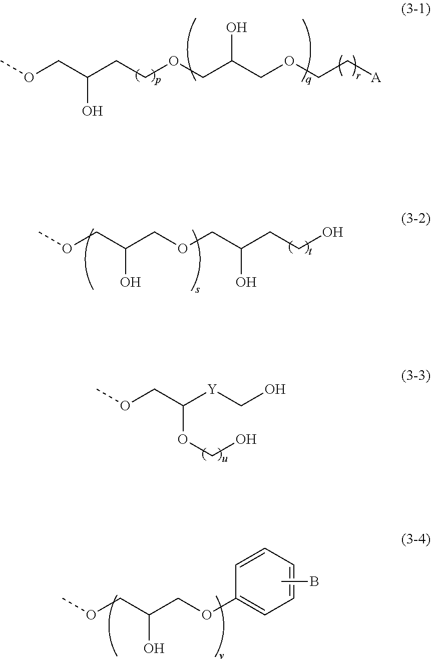
(in Formula (3-1), p represents an integer of 0 to 3, r represents an integer of 1 to 5, and a total value of p and r is 1 to 5; q represents an integer of 0 to 2; and A represents a polar group)
(in Formula (3-2), s represents an integer of 0 to 2, and t represents an integer of 1 to 5)
(in Formula (3-3), u represents an integer of 2 to 4; Y represents a linear alkylene group which may have an ether oxygen, or a single bond; and in a case where Y represents the alkylene group, a total number of carbon atoms and oxygen atoms contained in Y is 1 to 5)
(in Formula (3-4), v represents an integer of 1 to 3; and B represents a polar group in which a hydrogen at any position of a benzene ring is substituted).

4. The fluorine-containing ether compound according to claim 1,
wherein a total number of the polar groups contained in R¹ and the polar groups contained in R⁴ in Formula (1) is 4 to 6.

5. The fluorine-containing ether compound according to claim 1,
wherein R¹ and R⁴ in Formula (1) are the same as each other.

6. The fluorine-containing ether compound according to claim 1,
wherein at least one of the x pieces of R³'s in Formula (1) is represented by Formula (2-1).

7. The fluorine-containing ether compound according to claim 1,
wherein the (x+1) pieces of R²'s in Formula (1) are each independently a perfluoropolyether chain represented by Formula (4), 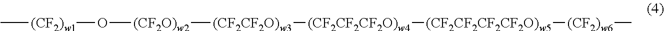
(in Formula (4), w2, w3, w4, and w5 each indicate an average degree of polymerization and each independently represent 0 to 20, provided that all of w2, w3, w4, and w5 are not 0 at the same time; w1 and w6 each indicate an average value of the number of CF₂'s and each independently represent 1 to 3; and (CF₂O), (CF₂CF₂O), (CF₂CF₂CF₂O), and (CF₂CF₂CF₂CF₂O), which are repeating units in Formula (4) are not particularly limited in the sequence order).

8. The fluorine-containing ether compound according claim 1,
wherein the (x+1) pieces of R²'s in Formula (1) are each independently any one selected from perfluoropolyether chains represented by Formulae (4-1) to (4-4), 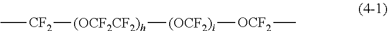
(in Formula (4-1), h and i each indicate an average degree of polymerization, h represents 1 to 20, and i represents 0 to 20) 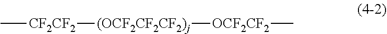
(in Formula (4-2), j indicates an average degree of polymerization and represents 1 to 15) 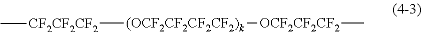
(in Formula (4-3), k indicates an average degree of polymerization and represents 1 to 1) 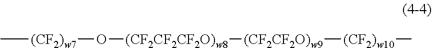
(in Formula (4-4), w8 and w9 each indicate an average degree of polymerization and each independently represent 1 to 20; and w7 and w10 are each an average value indicating the number of CF₂'s and each independently represent 1 or 2).

9. The fluorine-containing ether compound according to claim 1,
wherein the fluorine-containing ether compound has a number-average molecular weight in a range of 500 to 10,000.

10. A lubricant for a magnetic recording medium, comprising:
the flourine-containing ether compound according to claim 1.

11. A magnetic recording medium comprising, in the following order, on a substrate, at least:
a magnetic layer,
a protective layer, and
a lubricating layer,
wherein the lubricating layer includes the fluorine-containing ether compound according to claim 1.

12. The magnetic recording medium according to claim 11,
wherein an average film thickness of the lubricating layer is 0.5 nm to 2.0 nm.
